SEQUENCE LISTING (2 sequences)

SEQ ID NO: 1 - protein, 1153 aa, Artificial Sequence
  Integrin alpha-M isoform 1 precursor
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGQRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 2 - protein, 1152 aa, Artificial Sequence
  Integrin alpha-M isoform 2 precursor
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

(19) United States
(12) Patent Application Publication
(10) Pub. No.: US 2022/0296108 A1
(43) Pub. Date: Sep. 22, 2022

(54) METHOD AND APPARATUS FOR QUANTITATION OF MICROCIRCULATION

(71) Applicants: Seoul National University Hospital, Seoul (KR); Korea Advanced Institute of Science and Technology, Daejeon (KR)

(72) Inventors: Inwon Park, Gyeonggi-do (KR); Pilhan Kim, Daejeon (KR)

(21) Appl. No.: 17/613,278

(22) PCT Filed: Jan. 10, 2020
(86) PCT No.: PCT/KR2020/000549

§ 371 Date: May 31, 2022

(30) Foreign Application Priority Data
May 24, 2019 (KR) 10-2019-0061415
May 24, 2019 (KR) 10-2019-0061416
May 24, 2019 (KR) 10-2019-0061417

(51) Int. Cl. A61B 5/026; A61B 5/1455; A61B 5/0275

(52) U.S. Cl. CPC A61B 5/0261; A61B 5/14556; A61B 5/0275

(57) ABSTRACT

The present disclosure relates to a method for quantitation of microcirculation in a subject, in which a functional capillary ratio is calculated from a plurality of motion images of target factors over time in a first blood stream passing through the capillaries of the subject, and to an apparatus for measuring microcirculation in a subject. The present disclosure also relates to a method for providing information on microcirculatory disorder in a subject, in which a dynamic element in target factors is analyzed from a plurality of motion images of the target factors over time in a second blood stream passing through the capillaries of the subject, and an apparatus for diagnosis of microcirculatory disorder in a subject. The present disclosure also relates to a composition for prevention or treatment of lung injury, which contains an inhibitor against the expression or activity of macrophage-1 antigen (Mac-1) in neutrophils within pulmonary capillaries and alleviates microcirculatory disorder in the lung, a screening method, and a method for providing information for diagnosis of lung injury and disorder. The composition according to an embodiment of the present disclosure can inhibit the expression or activity of macrophage-1 antigen in neutrophils within pulmonary capillaries to allow erythrocyteserythrocytes to smoothly pass through the pulmonary capillary, whereby gas exchange is increased in a subject suffering from pulmonary microcirculatory disorder and, thus, the microcirculatory disorder in the lung can be alleviated. Thus, the composition exhibits excellent effect as a composition for prevention or treatment of lung injury.

### TECHNICAL FIELD

This application claims priority to Korean Patent Application No. 10-2019-0061415 field on May 24, 2019, the entire contents of which are incorporated herein by reference. In addition, this application claims priority to Korean Patent Application No. 10-2019-0061416 field on May 24, 2019, the entire contents of which are incorporated herein by reference. In addition, this application claims priority to Korean Patent Application No. 10-2019-0061417 field on May 24, 2019, the entire contents of which are incorporated herein by reference.

This research was conducted by the Korea Advanced Institute of Science and Technology with the support of the Ministry of Health & Welfare's disease recovery technology development project, under the research title “Development of in-vivo microscopy for imaging of pathophysiology of pulmonary hypertension at cellular scale” (project number: HI15C0399030017). In addition, this research was conducted by the Korea Advanced Institute of Science and Technology with the support of the Ministry of Science and ICT's individual basic research project, under the research title “Ultrafast laser scanning intravital microscopy needle probe-based deep-tissue microvisualization technology for human disease pathophysiology analysis and diagnosis” (project number: NRF-2017R1E1A1A01074190).

The present specification discloses a method for quantitation of microcirculation in a subject, in which a functional capillary ratio is calculated from a plurality of motion images of target factors over time in a first blood stream passing through the capillaries of the subject, and an apparatus for measuring microcirculation in a subject. In addition, the present specification discloses a method for providing information on microcirculatory disorder in a subject, in which a dynamic element in target factors is analyzed from a plurality of motion images of the target factors over time in a second blood stream passing through the capillaries of the subject, and an apparatus for diagnosis of microcirculatory disorder in a subject. In addition, the present specification discloses a composition for prevention or treatment of lung injury, which contains an inhibitor against the expression or activity of macrophage-1 antigen (Mac-1) in neutrophils within pulmonary capillaries and alleviates microcirculatory disorder in the lung, a screening method, a method for providing information for diagnosis of lung injury and disorder, and a composition and a for diagnosis of pulmonary microcirculatory disorder.

### BACKGROUND ART

Microcirculation is the circulation of blood in small blood vessels such as arterioles, venules, capillaries, lymph capillaries, etc. It is the core of metabolism, where supply and discharge of materials occur. The quantitation of microcirculation has been achieved by measurement of functional capillary density (FCD), by counting the number of functional capillaries by allocating 1 if a red blood cell that pass through the blood vessel within 30 seconds and allocating 0 otherwise. The existing method of measuring functional capillary density is limited in that the difference in functionality is not distinguished for the case where one red blood cell passes through one capillary in 30 seconds and the case where hundreds of red blood cell pass therethrough. In addition, since the pulmonary capillary has a network structure, there is limitation in calculating the density because it is difficult to define the beginning and end of each capillary. Furthermore, the method of measuring functional capillary density merely informs the functional capillary density of the microcirculatory system as a numerical value and fails to visualize the change in functional capillaries as images.

Sepsis is one of the foremost contributor to hospital deaths (Torio C M, Moore B J. National Inpatient Hospital Costs: The Most Expensive Conditions by Payer, 2013: Statistical Brief #204. Healthcare Cost and Utilization Project (HCUP) Statistical Briefs, Rockville (Md.), 2016; Hall M J, Levant S, DeFrances C J. Trends in inpatient hospital deaths: National Hospital Discharge Survey, 2000-2010. NCHS Data Brief 2013 (118): 1-8). It is a syndrome characterized by a dysregulated response of the host to invading pathogens, which involves hemodynamic alterations that lead to multiple life-threatening organ dysfunctions (Singer M, Deutschman C S, Seymour C W, Shankar-Hari M, Annane D, Bauer M, Bellomo R, Bernard G R, Chiche J D, Coopersmith C M, Hotchkiss R S, Levy M M, Marshall J C, Martin G S, Opal S M, Rubenfeld G D, van der Poll T, Vincent J L, Angus D C. The Third International Consensus Definitions for Sepsis and Septic Shock (Sepsis-3). JAMA 2016: 315(8): 801-810; Angus D C, van der Poll T. Severe sepsis and septic shock. N Engl J Med 2013: 369(9): 840-851). Among the organs damaged by sepsis, the lung is the first and most frequent organ to fail, and acute respiratory failure syndrome (ARDS) or acute lung injury (ALI) is one of the most critical prognostic factors for mortality in patients with sepsis (Lagu T, Rothberg M B, Shieh M S, Pekow P S, Steingrub J S, Lindenauer P K. Hospitalizations, costs, and outcomes of severe sepsis in the United States 2003 to 2007. Crit Care Med 2012: 40(3): 754-761). Despite intense research efforts aimed at treating sepsis-induced acute lung injury, no effective therapy aimed at microcirculation is available (Thompson B T, Chambers R C, Liu K D. Acute Respiratory Distress Syndrome. N Engl J Med 2017: 377(6): 562-572). Although it has been identified that dead space assessment could provide significant clinical data in acute lung injury (Nuckton T J, Alonso J A, Kallet R H, Daniel B M, Pittet J F, Eisner M D, Matthay M A. Pulmonary dead-space fraction as a risk factor for death in the acute respiratory failure syndrome. N Engl J Med 2002: 346(17): 1281-1286), until now, it has remained a hypothesis in terms of the impairment of the lung alveoli that are ventilated but not perfused. Admittedly, acute respiratory failure syndrome is a poorly understood syndrome with regard to the association between lung injury and microcirculation (Ryan D, Frohlich S, McLoughlin P. Pulmonary vascular dysfunction in ARDS. Ann Intensive Care 2014: 4: 28). Recently, a study reported the evidence of thrombi in the pulmonary vasculature which was limited as an ex-vivo study, and to date, the in-vivo process of neutrophil influx and the consequent disorder of pulmonary microcirculation remain to be investigated (Matthay M A, Ware L B, Zimmerman G A. The acute respiratory failure syndrome. J Clin Invest 2012: 122(8): 2731-2740; Yuan Y, Alwis I, Wu MCL, Kaplan Z, Ashworth K, Bark D, Jr., Pham A, McFadyen J, Schoenwaelder S M, Josefsson E C, Kile B T, Jackson S P. Neutrophil macroaggregates promote widespread pulmonary thrombosis after gut ischemia. Sci Transl Med 2017: 9(409)).

Unregulated recruitment and activation of neutrophils could induce organ injury through release of inflammatory mediators, including cytokines and reactive oxygen species (ROS) (Grommes J, Soehnlein O. Contribution of neutrophils to acute lung injury. Mol Med 2011: 17(3-4): 293-307; Matute-Bello G, Downey G, Moore B B, Groshong S D, Matthay M A, Slutsky A S, Kuebler W M, Acute Lung Injury in Animals Study G. An official American Thoracic Society workshop report: features and measurements of experimental acute lung injury in animals. Am J Respir Cell Mol Biol 2011: 44(5): 725-738). Yet, the existing knowledge on the detailed dynamic behavior of neutrophils in the pulmonary microcirculation is mostly limited to speculation gleaned from observations in the systemic circulation (Phillipson M, Kubes P. The neutrophil in vascular inflammation. Nat Med 2011: 17(11): 1381-1390). Because the diameter of neutrophils is greater than that of the pulmonary capillaries, neutrophils should be deformed to pass through the capillaries, which is a relatively time-consuming process (Doerschuk C M. Mechanisms of leukocyte sequestration in inflamed lungs. Microcirculation 2001: 8(2): 71-88). This process, referred to as neutrophil sequestration, was originally described for cells other than the freely circulating group of neutrophils within the lung and has been observed, to some extent, using macroscopic radiolabeling imaging devices (MacNee W, Selby C. New perspectives on basic mechanisms in lung disease; Neutrophil traffic in the lungs: role of hemodynamics, cell adhesion, and deformability. Thorax 1993: 48(1): 79-88). Indeed, previous studies have demonstrated neutrophil sequestration in lung capillaries; however, the mechanism of how the neutrophil sequestration event leads to acute lung injury or acute respiratory failure syndrome remains unknown (Kuebler W M, Borges J, Sckell A, Kuhnle G E, Bergh K, Messmer K, Goetz A E. Role of L-selectin in leukocyte sequestration in lung capillaries in a rabbit model of endotoxemia. Am J Respir Crit Care Med 2000: 161(1): 36-43; Lien D C, Henson P M, Capen R L, Henson J E, Hanson W L, Wagner W W, Jr., Worthen G S. Neutrophil kinetics in the pulmonary microcirculation during acute inflammation. Lab Invest 1991: 65(2): 145-159). Therefore, when considering the importance and obscurity of the pulmonary microcirculation in acute lung injury or acute respiratory failure syndrome, understanding the changes in the pulmonary microcirculation including the dynamic behavior of neutrophils is imperative for elucidation of the pathophysiology, which may lead to novel treatment strategies for sepsis-induced acute lung injury or acute respiratory failure syndrome (Looney M R, Bhattacharya J. Live imaging of the lung. Annu Rev Physiol 2014: 76: 431-445).

The inventors of the present disclosure have studied on a method for quantitation of microcirculation in a subject based on area rather than density, and have completed the present disclosure. To investigate pulmonary microcirculation in sepsis-induced lung injury, the inventors of the present disclosure used a custom-designed video-rate laser scanning confocal microscope in combination with a micro-suction-based lung imaging window (Kim P, Puoris′haag M, Cote D, Lin C P, Yun S H. In vivo confocal and multiphoton microendoscopy. J Biomed Opt 2008: 13(1): 010501; Han S, Lee S J, Kim K E, Lee H S, Oh N, Park I, Ko E, Oh S J, Lee Y S, Kim D, Lee S, Lee D H, Lee K H, Chae S Y, Lee J H, Kim S J, Kim H C, Kim S, Kim S H, Kim C, Nakaoka Y, He Y, Augustin H G, Hu J, Song P H, Kim Y I, Kim P, Kim I, Koh G Y. Amelioration of sepsis by TIE2 activation-induced vascular protection. Sci Transl Med 2016: 8(335): 335ra355). Using the intravital lung imaging system, they directly identified the alteration of microcirculatory perfusion in a sepsis-induced acute lung injury (ALI) model, and completed the present disclosure. Furthermore, for development of a composition for preventing or treating lung injury, the inventors of the present disclosure observed the neutrophils of a model having pulmonary microcirculatory disorder using the custom-designed video-rate laser scanning confocal microscope and completed the present disclosure by identifying a target for improvement of pulmonary microcirculatory disorder in the neutrophils.

### DISCLOSURE

### Technical Problem

In an aspect, the present disclosure is directed to providing a method and an apparatus for quantitation of microcirculation in a subject based on a functional capillary ratio (FCR), which is the ratio of functional capillary area measured from a plurality of motion images of target factors over time in a first blood stream passing through the capillaries of the subject to the total capillary area, and a computer program executing the method.

In another aspect, the present disclosure is directed to providing a method and an apparatus for providing information for diagnosis of microcirculatory disorder in a subject, which allows fast and accurate diagnosis of microcirculatory disorder in a subject using a functional capillary ratio (FCR) of the subject calculated by the method for quantitation of microcirculation described above, and a computer program and a system executing the method.

In another aspect, the present disclosure is directed to providing a method for providing information for diagnosis of microcirculatory disorder in a subject by analyzing dynamic elements of target factors such as sequestration time, track displacement length, track length, track velocity or track meandering index from a plurality of motion images of the target factors over time in a second blood stream passing through the capillaries of the subject, and an apparatus for diagnosis of microcirculatory disorder.

In another aspect, the present disclosure is directed to providing a composition for prevention or treatment of lung injury, which is capable of alleviating microcirculatory disorder in the lung by inhibiting the expression or activity of macrophage-1 antigen (Mac-1) in neutrophils within pulmonary capillaries, thereby allowing erythrocyteserythrocytes to smoothly pass through the pulmonary capillaries and increasing gas exchange in a subject suffering from pulmonary microcirculatory disorder, and a method for screening a substance for preventing or treating lung injury.

In another aspect, the present disclosure is directed to providing a method for providing information useful for diagnosis of pulmonary microcirculatory disorder, and a composition and a kit for diagnosis of pulmonary microcirculatory disorder.

### Technical Solution

In an aspect, the present disclosure provides a method for quantitation of microcirculation in a subject, which includes: a step of obtaining a plurality of motion images of target factors over time in a first blood stream passing through the capillaries of the subject; a step of measuring functional capillary area in which the target factors move in the first blood stream from the plurality of motion images; and a step of calculating functional capillary ratio (FCR) according to Formula 1.

Functional capillary ratio=functional capillary area/total capillary area. [Formula 1]

In another aspect, the present disclosure provides an apparatus for measuring microcirculation in a subject, which acquires quantitative data on the microcirculation in the subject based on a plurality of motion images of target factors over time in a first blood stream passing through the capillaries of the subject according to Formula 1. Specifically, the apparatus may include: an imaging unit imaging target factors in a first blood stream passing through the capillaries of the subject; and a measuring unit acquiring quantitative data on the microcirculation in the subject according to Formula 1 based on the images imaged by the imaging unit.

In another aspect, the present disclosure provides a method for providing information for diagnosis of microcirculatory disorder in a subject, which includes a step of acquiring information for diagnosing microcirculatory disorder in the subject from the functional capillary ratio (FCR) calculated by the method for quantitation of microcirculation in a subject described above.

In another aspect, the present disclosure provides a computer program stored in a computer-readable medium, which is associated with a hardware and executes the method for quantitation of microcirculation or the method for providing information for diagnosis of microcirculatory disorder in a subject.

In another aspect, the present disclosure provides a method for providing information for diagnosis of microcirculatory disorder, which includes: a step of obtaining a plurality of motion images of target factors over time in a second blood stream flowing through the capillaries of the subject; a step of analyzing one or more dynamic element selected from a group consisting of sequestration time, track displacement length, track length, track velocity and track meandering index of the target factors in the second blood stream from the plurality of motion images; and a step of acquiring information for diagnosis of microcirculatory disorder in the subject from the dynamic element analysis result.

In another aspect, the present disclosure provides an apparatus for diagnosis of microcirculatory disorder, which includes: an imaging unit imaging target factors in a second blood stream passing through the capillaries of a subject; and an analysis unit analyzing one or more dynamic element selected from a group consisting of sequestration time, track displacement length, track length, track velocity and track meandering index of the target factors in the second blood stream based on the plurality of motion images imaged by the imaging unit.

In another aspect, the present disclosure provides a composition for preventing, alleviating or treating lung injury, which contains an inhibitor against the expression or activity of macrophage-1 antigen (Mac-1) in neutrophils within pulmonary capillaries as an active ingredient, and prevents, alleviates or treats lung injury through alleviation of microcirculatory disorder in the lung.

In another aspect, the present disclosure provides a method for screening a substance for preventing, alleviating or treating lung injury, which includes: (a) a step of preparing a lung injury model; (b) a step of the lung injury model with a test substance; (c) a step of measuring the change in the expression or activity of macrophage-1 antigen (Mac-1) in neutrophils within pulmonary capillaries of the lung injury model caused by the test substance; and (d) a step of identifying whether the test substance increases a functional capillary ratio, which is the ratio of the area of functional capillaries through which erythrocyteserythrocytes pass to the total capillary area of the lung injury model.

In another aspect, the present disclosure provides a method for providing information for diagnosis of pulmonary microcirculatory disorder, which includes: a step of measuring the expression or activity of macrophage-1 antigen (Mac-1) in neutrophils isolated from the pulmonary capillaries of a test subject; and a step of identifying a functional capillary ratio, which is the ratio of the area of functional capillaries through which erythrocyteserythrocytes pass to the total capillary area of the lung of the test subject.

In another aspect, the present disclosure provides a composition for diagnosis of pulmonary microcirculatory disorder, which contains a reagent for detecting mRNAs or proteins of macrophage-1 antigen (Mac-1) in neutrophils within pulmonary capillaries.

In another aspect, the present disclosure provides a kit for diagnosis of pulmonary microcirculatory disorder, which contains a reagent for detecting mRNAs or proteins of macrophage-1 antigen (Mac-1) in neutrophils within pulmonary capillaries.

### Advantageous Effects

The present disclosure relates to quantification of microcirculation of a subject based on a functional capillary ratio (FCR), or the ratio of functional capillary area, which is the ratio of functional capillary area measured from a plurality of motion images of target factors over time in a first blood stream passing through the capillaries of the subject to the total capillary area. Because microcirculation can be quantified based on area rather than density, the area where through which one red blood cell passes and the area where through which a plurality of erythrocyteserythrocytes pass can be distinguished.

Through this, microcirculation can be quantified more easily, conveniently and accurately because the space (area) through which erythrocyteserythrocytes pass actually can be reflected, and it is possible to quantify the microcirculation network which is difficult to quantify based on density.

In addition, because the ratio of the functional capillary area to the total capillary area can be visually identified with one image, the location of functional capillaries, i.e., the capillaries through which more erythrocyteserythrocytes pass, can be identified conveniently, and microcirculatory disorder can be identified accurately and quickly based on the quantification result.

In addition, the present disclosure relates to a method for providing information for diagnosis of microcirculatory disorder in a subject by analyzing a dynamic element selected from a group consisting of sequestration time, track displacement length, track length, track velocity and track meandering index of target factors in a second blood stream flowing through the capillaries of the subject from a plurality of motion images of the target factors over time, and an apparatus for diagnosis of microcirculatory disorder. The method and apparatus provide the advantage that microcirculatory disorder in a subject can be diagnosed more accurately and quickly by acquiring information on the motion of neutrophils within capillaries easily and conveniently.

In addition, the composition according to an embodiment of the present disclosure exhibits superior effect as a composition for prevention or treatment of lung injury because it can alleviate microcirculatory disorder in the lung by inhibiting the expression or activity of macrophage-1 antigen (Mac-1) in neutrophils within pulmonary capillaries, thereby allowing erythrocyteserythrocytes to smoothly pass through the pulmonary capillaries and increasing gas exchange in a subject suffering from pulmonary microcirculatory disorder. In addition, it allows faster, more convenient and more accurate diagnosis of pulmonary microcirculatory disorder through measurement of the expression or activity of macrophage-1 antigen in neutrophils isolated from the pulmonary capillaries.

### BRIEF DESCRIPTION OF DRAWINGS

FIG. 1 schematically shows a procedure of intravital lung imaging for visualization of pulmonary microcirculation with adoptive transfer of DiD-labeled erythrocyteserythrocytes according to an exemplary embodiment of the present disclosure.

FIG. 2 shows sequential images obtained by imaging the pulmonary microcirculation of a lung injury mouse model using an imaging system according to an exemplary embodiment of the present disclosure. In FIG. 2, Time sequence indicates images of the moving fluorescence-stained erythrocyteserythrocytes at different times (0.000 sec, 0.033 sec and 0.066 sec), and Merge indicates a merged image. In FIG. 2, the green color (lines or areas) indicates the fluorescence-stained vasculature (total capillaries) stained with a dextran dye in the pulmonary capillaries of the mouse model according to an exemplary embodiment of the present disclosure, and the red color (dots) indicates the DiD-labeled erythrocyteserythrocytes (functional capillaries) in the pulmonary capillaries of the mouse model according to an exemplary embodiment of the present disclosure.

FIG. 3 shows the ratio of functional capillary area in which erythrocyteserythrocytes pass at different times (90 frames, 180 frames, 360 frames and 600 frames) in a lung injury mouse model according to an exemplary embodiment of the present disclosure to the total capillary area. In FIG. 3, the green images (Capillary, Tie2) show the fluorescence-stained vasculature (total capillaries) stained with a dextran dye in the pulmonary capillaries of the mouse model according to an exemplary embodiment of the present disclosure, the red images (Functional, DiD-RBC) show the DiD-labeled erythrocyteserythrocytes (functional capillaries) in the pulmonary capillaries of the mouse model according to an exemplary embodiment of the present disclosure, and Merge indicates merged images.

FIG. 4 shows the ratio of functional capillary area, i.e., the area in which erythrocyteserythrocytes pass in a lung injury mouse model according to an exemplary embodiment of the present disclosure at different times. In FIG. 4, the x-axis indicates the number of time-projected frames, and the y-axis indicates the ratio of functional capillary (%).

FIG. 5 shows a result of imaging total capillaries and functional capillaries of a control group model (PBS) and a lung injury mouse model (LPS) according to an exemplary embodiment of the present disclosure. In FIG. 5, the green images (Capillary) show the fluorescence-stained vasculature (total capillaries) stained with a dextran dye in pulmonary capillaries, the red images (Functional) show DiD-labeled erythrocyteserythrocytes (functional capillaries, Functional), and Merge indicates merged images.

FIGS. 6A-6D show the total capillary area (FIG. 6A), functional capillary ratio (FCR, FIG. 6B), partial pressure of arterial oxygen (FIG. 6C) and partial pressure of arterial carbon dioxide (FIG. 6D) of a control group model (PBS) and a lung injury mouse model (LPS) according to an exemplary embodiment of the present disclosure.

FIG. 7 shows a result of imaging the pulmonary microcirculation of a LysM^{GFP/+} mouse model using an imaging system according to an exemplary embodiment of the present disclosure and processing the obtained images according to an exemplary embodiment of the present disclosure. In FIG. 7, the green color (LysM^{GFP/+}) indicates neutrophils, the red color (*, TMR Dextran) indicates pulmonary capillaries. The scale bars in FIG. 7 are 10 μm.

FIG. 8 shows a result of imaging the motion of neutrophils in an ALI mouse model (LPS) and a control group model (PBS) according to an exemplary embodiment of the present disclosure using an imaging system according to an exemplary embodiment of the present disclosure and processing the obtained images. In FIG. 8, the red color (Ly6G) indicates neutrophils, and the green color (FITC Dextran) indicates pulmonary capillaries. The Magnified spot images consist of averaged imaging up to 30 frames, and the dashed arrows indicate the direction of flow. The white arrowheads (bright shade, white color) indicate entrapped neutrophils, and the yellow arrowhead (dark shade, gray color) indicate obstructed capillaries with no flow. In FIG. 8, the scale bars are 100 μm for the Wide field images and 20 μm for the Magnified spot images.

FIG. 9 compares the number of neutrophils per unit area (512×512 μm) (field) for an ALI mouse model (LPS) and a control group model (PBS) according to an exemplary embodiment of the present disclosure.

FIG. 10A shows a result of time-lapse imaging of the pulmonary microcirculation of lung injury mouse models (LPS 3h mouse model and LPS 6h mouse model) and a control group model (PBS) according to an exemplary embodiment of the present disclosure for 30 minutes at slow rate and imaging the motion of tracked neutrophils (Ly6G+ cells) (Track). In FIG. 10A, the scale bars are 100 μm. In FIG. 10A, the red color (Ly6G, white squares) indicates neutrophils and the green color (FITC Dextran, gray-shaded lines or planes in Neutrophil spot images) indicates pulmonary capillaries.

FIG. 10B shows a result of overlaying the track of neutrophils (Ly6G+ cells) in FIG. 10A. Each track of the neutrophils is plotted from the central point and shows XY track displacement. The scale bars are 10 μm.

FIG. 11 shows the number of tracks for the lung injury mouse models (LPS 3h mouse model and LPS 6h mouse model) and a control group model (PBS) according to an exemplary embodiment of the present disclosure, shown in FIGS. 10A and 10B, depending on sequestration time.

FIGS. 12A-12E show the dynamic behavior of neutrophils, i.e., sequestration time (FIG. 12A), track displacement length (FIG. 12B), track length (FIG. 12C), track velocity (FIG. 12D) and track meandering index (FIG. 12E), in lung injury mouse models (LPS 3h mouse model and LPS 6h mouse model) and a control group model (PBS) according to an exemplary embodiment of the present disclosure.

FIG. 13 shows a result of imaging the pulmonary microcirculation of the neutrophils (Ly6G+ cells) of an ALI mouse model according to an exemplary embodiment of the present disclosure in real time. In FIG. 13, the dashed arrows indicate blood flow, the yellow arrowheads (dark shade, gray color) indicate previously entrapped neutrophils, and the white arrowheads (bright shade, and white color) indicate newly appeared neutrophils obstructing capillaries, leading to dead space formation inside the capillaries. Also, in FIG. 13, the dashed lines indicate the dead space formed in the capillaries. In FIG. 13, the scale bars are 20 μm.

FIG. 14 shows a result of imaging the pulmonary microcirculation of the neutrophils (Ly6G+ cells) of an ALI mouse model according to an exemplary embodiment of the present disclosure in real time. In FIG. 14, the red color (Ly6G+, dark shade) indicates neutrophils, and the green color (FITC Dextran, bright shade) indicates pulmonary capillaries. The left image in FIG. 14 shows a result of intravital imaging of thrombus formation inside capillaries (scale bar: 20 μm), the central image in FIG. 14 shows a result of intravital imaging of thrombus formation in arterioles (scale bar: 100 μm), and the right image in FIG. 14 is a magnified image of the blue dashed square in the central image in FIG. 14 (scale bar: 20 μm).

FIG. 15 shows time-lapse images obtained by conducting intravital imaging of cluster formation by neutrophils (Ly6G+ cells) in the branching region of arterioles connected to the capillaries of an ALI mouse model according to an exemplary embodiment of the present disclosure using a customized video-rate laser scanning confocal microscopy system by a method according to an exemplary embodiment of the present disclosure for 10 minutes at slow rate. In FIG. 15, the red color (Ly6G, bright shade) indicates neutrophils, and the green color (FITC Dextran, dark shade) indicates pulmonary capillaries. Time elapsed is indicated as MM:SS (minute:second), and the scale bars are 20 μm.

FIG. 16 shows images obtained by imaging the pulmonary microcirculation of an ALI mouse model having DiD-labeled erythrocytes according to an exemplary embodiment of the present disclosure for 10 minutes at a slow rate using a method according to an exemplary embodiment of the present disclosure, and the track path of the DiD-labeled erythrocytes obtained through tracking of neutrophils according to an exemplary embodiment of the present disclosure. In FIG. 16, the magenta color (Ly6G, bright shade) indicates neutrophils, the green color (FITC Dextran, dark shade) indicates pulmonary capillaries, the white dashed circles indicate microcirculatory dead spaces, the white arrow indicates the direction of blood flow, and the scale bars are 100 μm.

FIG. 17 shows a result of investigating the production of reactive oxygen species in neutrophils of an ALI mouse model (LPS) and a control group model (PBS) according to an exemplary embodiment of the present disclosure through staining with DHE. In FIG. 17, the green color (FITC Dextran) indicates pulmonary capillaries, the red color (Ly6G) indicates neutrophils, the blue color (DHE) indicates reactive oxygen species (ROS), and the scale bars are 50 μm.

FIG. 18A compares the number of reactive oxygen species-generating neutrophils (ROS+ Ly6G+) of an ALI mouse model (LPS) and a control group model (PBS) according to an exemplary embodiment of the present disclosure per unit area (512×512 μm).

FIG. 18B compares the ratio of reactive oxygen species-generating neutrophils (ROS+ Ly6G+) to total neutrophils (Ly6G+) of an ALI mouse model (LPS) and a control group model (PBS) according to an exemplary embodiment of the present disclosure.

FIG. 19 schematically shows a process of preparing a neutrophil-depleted lung injury mouse model (N-Dep+LPS mouse model) using an acute lung injury mouse model (ALI mouse model) according to an exemplary embodiment of the present disclosure.

FIG. 20 shows a result of imaging the pulmonary microcirculation of a control mouse model (PBS), an ALI mouse model (LPS) and neutrophil-depleted models (N-Dep mouse model and N-Dep+LPS mouse model) according to an exemplary embodiment of the present disclosure using an imaging system according to an exemplary embodiment of the present disclosure and processing the obtained images. In FIG. 20, the green color (Capillary, TMR Dextran) indicates anatomical capillaries, the red color (Functional, DiD-RBC) indicates functional capillaries, the magenta color (LysM, LysM^{GFP/+}) indicates neutrophils, the white asterisks (*) indicate dead spaces, and the white arrowheads indicate entrapped or sequestered neutrophils. In FIG. 20, the Merge images were obtained by merging the images of the anatomical capillaries, functional capillaries and neutrophils, and the Magnified images were obtained by merging the images of the anatomical capillaries and neutrophils. The scale bars are 20 μm in the Magnified images of FIG. 20 and 100 μm in other images.

FIG. 21A shows the functional capillary ratio (FCR) of a control mouse model (PBS), an ALI mouse model (LPS), neutrophil-depleted models (N-Dep mouse model and N-Dep+LPS mouse model) according to an exemplary embodiment of the present disclosure.

FIG. 21B shows the number of neutrophils of a control mouse model (PBS), an ALI mouse model (LPS) and neutrophil-depleted models (N-Dep mouse model and N-Dep+LPS mouse model) according to an exemplary embodiment of the present disclosure per unit area (512×512 μm).

FIG. 22 schematically shows a process of isolating neutrophils from left ventricle (LV) and the lung of a mouse model according to an exemplary embodiment of the present disclosure.

FIG. 23 shows a result of conducting flow cytometry for neutrophils isolated from left ventricle (LV, red) and the lung (blue) of a mouse model according to an exemplary embodiment of the present disclosure.

FIGS. 24A-24D compare the expression level of CD11a, CD11 b, CD18 and CD62L in neutrophils isolated from left ventricle (LV) and the lung of a control (PBS) mouse model and an ALI mouse model (LPS) according to an exemplary embodiment of the present disclosure. In FIGS. 24A-24D, MFI means mean fluorescence intensity.

FIGS. 25A and 25B shows a result of visualizing sequestered neutrophils of a control (PBS) mouse model and an ALI mouse model (LPS) according to an exemplary embodiment of the present disclosure and the expression of CD11 b and CD18 on the surface of the neutrophils in vivo. In FIG. 25A and FIG. 25B, the red color (Ly6G+) indicates neutrophils, and the green color (CD11b or CD18) indicates the expression of CD11 b or CD18 on the surface of the neutrophils. The Merge images are imaged obtained by merging the images of the neutrophils and the CD11 b expression or the images of the neutrophils and the CD18 expression. In FIGS. 25A and 25B, the scale bars are 100 μm.

FIGS. 26A-26D compare the number of neutrophils expressing CD11b or CD18 of an ALI mouse model (LPS) and a control mouse model (PBS) according to an exemplary embodiment of the present disclosure. FIG. 26A shows the number of neutrophils expressing CD11b per unit area (512×512 μm), FIG. 26B shows the ratio of the neutrophils expressing CD11b to the total neutrophils, FIG. 26C shows the number of neutrophils expressing CD18 per unit area (512×512 μm), and FIG. 26D shows the ratio of the neutrophils expressing CD18 to the total neutrophils.

FIG. 27 shows a result of imaging the pulmonary microcirculation of a CLP mouse model (Fc), an anti-Mac-1 mouse model (Anti-CD11b), an abciximab mouse model (Abc) and a normal mouse model (Sham) according to an exemplary embodiment of the present disclosure using an imaging system according to an exemplary embodiment of the present disclosure and processing the obtained images. In FIG. 27, the green color (Capillary, TMR Dextran) indicates anatomical capillaries, the red color (Functional, DiD-RBC) indicates functional capillaries, the magenta color (Ly6G) indicates neutrophils, and the white asterisks (*) indicate dead spaces. In FIG. 27, the Merge images were obtained by merging the images of the anatomical capillaries, functional capillaries and neutrophils, and the scale bars are 100 μm.

FIG. 28A compares the functional capillary ratio (FCR) of a CLP mouse model (Fc), an anti-Mac-1 mouse model (Anti-CD11b), an abciximab mouse model (Abc) and a normal mouse model (Sham) according to an exemplary embodiment of the present disclosure.

FIG. 28B compares the number of neutrophils (Ly6G+ cells) of a CLP mouse model (Fc), an anti-Mac-1 mouse model (Anti-CD11b), an abciximab mouse model (Abc) and a normal mouse model (Sham) according to an exemplary embodiment of the present disclosure.

FIG. 29 shows a result of imaging the pulmonary microcirculation of a CLP mouse model before administration of abciximab (pre-Abc) and a CLP mouse model after administration of abciximab (post-Abc) according to an exemplary embodiment of the present disclosure using an imaging system according to an exemplary embodiment of the present disclosure and processing the obtained images. In FIG. 29, the green color (Capillary, FITC Dextran) indicates anatomical capillaries, the red color (Functional, DiD-RBC) indicates functional capillaries, the magenta color (LysM, LysM^{GFP/+}) indicates neutrophils, and the white arrowheads indicate the restoration of red blood cell perfusion. In FIG. 29, the Merge images were obtained by merging the images of anatomical capillaries, functional capillaries and neutrophils, and the scale bars are 100 μm.

FIG. 30 compares the functional capillary ratio (FCR) of a CLP mouse model before administration of abciximab (pre-Abc) and a CLP mouse model after administration of abciximab (post-Abc) according to an exemplary embodiment of the present disclosure.

FIG. 31A and FIG. 31B compare the oxygen partial pressure and carbon dioxide partial pressure in the arterial blood of a normal mouse model (Sham), a CLP mouse model before administration of abciximab (Fc) and a CLP mouse model after administration of abciximab (Abc) according to an exemplary embodiment of the present disclosure.

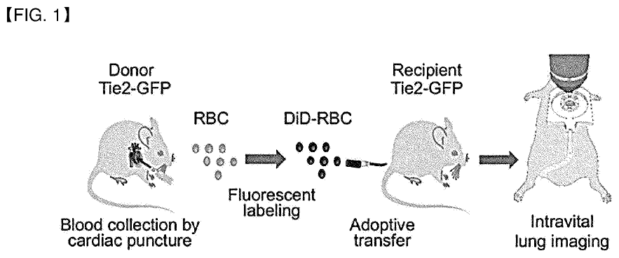

### BEST MODE

In an aspect of the present specification, the terms “unit”, “module”, “device”, “system”, etc. may refer to not only a hardware but also a combination of softwares executed by the hardware. For example, the hardware may be a CPU or other data-processing devices including processors. And, the software executed by the hardware may be a process, an object, an executable file, a thread of execution, a program such as a calculation program, etc.

In an aspect of the present specification, “microcirculation” is the circulation of blood in small blood vessels such as arterioles, venules, capillaries, lymph capillaries, etc. It is the core of metabolism, where supply and discharge of materials occur. In an aspect of the present disclosure, the microcirculation may be quantified based on a functional capillary area measured form of a plurality of motion images of target factors in a first blood stream, information for diagnosis of microcirculatory disorder in the subject may be provided, or the microcirculatory disorder of a subject may be diagnosed by analyzing a dynamic element in target factors in a second blood stream from a plurality of motion images of the target factors in the second blood stream, although the present specification is not limited thereto. And, in an aspect of the present disclosure, the microcirculation may be, for example, microcirculation in the lung, microcirculation in the eye, microcirculation in the kidney or microcirculation in skin, and the skin may be hand, foot, etc., although not being limited thereto.

In an aspect of the present specification, the subject for quantitation of microcirculation or diagnosis of microcirculatory disorder may be any subject without limitation. Specifically, the subject may be a non-human animal such as monkey, dog, cat, rabbit, guinea pig, rat, mouse, cow, sheep, pig, goat, etc. or human, although not being limited thereto. In addition, the subject may be a subject having microcirculatory disorder, capillary circulatory disorder or peripheral circulatory disorder, although not being limited thereto.

In an aspect of the present specification, the capillary of the subject is not particularly limited as long as microcirculation may be quantified based on a functional capillary area measured form of a plurality of motion images of target factors in a first blood stream, information for diagnosis of microcirculatory disorder in the subject may be provided, or the microcirculatory disorder of the subject may be diagnosed by analyzing a dynamic element in target factors in a second blood stream from a plurality of motion images of the target factors in the second blood stream, and may be one or more capillary selected from a group consisting of the lung, kidney, skin and eye of the subject, although not being limited thereto.

In an aspect of the present specification, the plurality of motion images over time may be a plurality of images imaged with time intervals of 1/900 to 1 second. For example, when the plurality of images are an image (M) at one time point (T) and images (M−1, M+1) at time points (T−1, T+1) before and after the time point (T) of the same time interval (t), because the three images (M−1, M, M+1) are respectively images at first time point (T−1), second time point (T) and third time point (T+1) with the same time interval (t), a flow path of target factors in a first blood stream or a second blood stream passing through the capillaries of the subject may occur over time in the three images (M−1, M, M+1). Therefore, quantitative data about microcirculation can be acquired by measuring the functional capillary area in which the target factor pass in the first blood stream. Specifically, the functional capillary area may be measured by identifying the same target factors in the three images (M−1, M, M+1) imaged with the time interval (t). Also, information for diagnosis of microcirculatory disorder may be provided by analyzing dynamic elements of the target factor in the second blood stream, e.g., the sequestration time, track displacement length, track length, track velocity or track meandering index of the target factors. Specifically, the dynamic elements of the target factors may be analyzed by identifying the same target factors in the three images (M−1, M, M+1) imaged with the time interval (t).

In an aspect of the present specification, the time interval (t) of the three images (M−1, M, M+1) may be from 1/900 second to 1 second, specifically from 1/300 second to ⅓ second, more specifically 1/900 second or longer, 1/800 second or longer, 1/700 second or longer, 1/600 second or longer, 1/500 second or longer, 1/400 second or longer, 1/300 second or longer, 1/200 second or longer, 1/100 second or longer, 1/90 second or longer, 1/80 second or longer, 1/70 second or longer, 1/60 second or longer, 1/50 second or longer, 1/45 second or longer, 1/40 second or longer, 1/35 second or longer, 1/30 second or longer, 1/25 second or longer, 1/20 second or longer, 1/15 second or longer, 1/10 second or longer or ⅕ second or longer, and 1 second or shorter, ⅕ second or shorter, 1/10 second or shorter, 1/15 second or shorter, 1/20 second or shorter, 1/25 second or shorter, 1/30 second or shorter, 1/35 second or shorter, 1/40 second or shorter, 1/45 second or shorter, 1/50 second or shorter, 1/60 second or shorter, 1/70 second or shorter, 1/80 second or shorter, 1/90 second or shorter, 1/100 second or shorter, 1/200 second or shorter, 1/300 second or shorter, 1/400 second or shorter, 1/500 second or shorter, 1/600 second or shorter, 1/700 second or shorter, 1/800 second or shorter or 1/900 second or shorter. However, the time interval is not specially limited as long as microcirculation can be quantified from a plurality of motion images of the target factors in the first blood stream, or the dynamic elements of the target factor can be analyzed from the plurality of motion images of the target factors in the second blood stream.

In another aspect of the present specification, the plurality of motion images may be a plurality of images imaged at a frame rate or speed of 1-900 frames/second. The frame rate or speed may be specifically 3-300 frames/second, more specifically 1 frame/second or higher, 5 frames/second or higher, 10 frames/second or higher, 15 frames/second or higher, 20 frames/second or higher, 25 frames/second or higher, 30 frames/second or higher, 35 frames/second or higher, 40 frames/second or higher, 45 frames/second or higher, 50 frames/second or higher, 60 frames/second or higher, 70 frames/second or higher, 80 frames/second or higher, 90 frames/second or higher, 100 frames/second or higher, 200 frames/second or higher, 300 frames/second or higher, 400 frames/second or higher, 500 frames/second or higher, 600 frames/second or higher, 700 frames/second or higher or 800 frames/second or higher, and 900 frames/second or lower, 800 frames/second or lower, 700 frames/second or lower, 600 frames/second or lower, 500 frames/second or lower, 400 frames/second or lower, 300 frames/second or lower, 200 frames/second or lower, 100 frames/second or lower, 90 frames/second or lower, 80 frames/second or lower, 70 frames/second or lower, 60 frames/second or lower, 50 frames/second or lower, 45 frames/second or lower, 40 frames/second or lower, 35 frames/second or lower, 30 frames/second or lower, 25 frames/second or lower, 20 frames/second or lower, 15 frames/second or lower, 10 frames/second or lower or 5 frames/second or lower. However, the frame rate or speed is not specially limited as long as microcirculation can be quantified from a plurality of motion images of the target factors in the first blood stream, or the dynamic elements of the target factor can be analyzed from the plurality of motion images of the target factors in the second blood stream.

In an aspect of the present specification, the plurality of images may be images imaged by confocal scanning laser microscopy, fluorescence microscopy, two-photon microscopy or three-photon microscopy, although not being limited thereto.

In an aspect of the present specification, the functional capillary refers to a capillary where the function of the capillary, e.g., exchange of oxygen, carbon dioxide, nutrients and other substances between blood and tissue through diffusion, occurs actively. The functional capillary may be a capillary through which the target factors in a first blood stream such as leukocytes, erythrocytes, blood platelets, lymphocytes, etc. pass or a capillary through which the target factors in a second blood stream such as neutrophils, etc. pass. A higher ratio of the functional capillary to the total capillary means that microcirculation in the subject is smooth or there is no microcirculatory disorder.

Hereinafter, the present disclosure is described in detail.

In an aspect, the present disclosure provides a method for quantitation of microcirculation in a subject, which includes: a step of obtaining a plurality of motion images of target factors over time in a first blood stream passing through the capillaries of the subject; a step of measuring functional capillary area in which the target factors move in the first blood stream from the plurality of motion images; and a step of calculating functional capillary ratio (FCR) according to Formula 1.

Functional capillary ratio=functional capillary area/total capillary area. [Formula 1]

The quantitation of microcirculation in a subject means the quantification of the degree of microcirculation in the subject.

The method for quantitation of microcirculation in a subject may include a step of obtaining a plurality of motion images of target factors over time in a first blood stream passing through the capillaries of the subject.

The target factors in the first blood stream are factors that pass through the microcirculatory capillaries of the subject. The quantitative data about the microcirculation may be acquired by measuring the area traveled by the target factors in the first blood stream, e.g., functional capillary area, over time from the plurality of motion images. The target factors in the first blood stream may be the factors that pass through the capillaries of the subject through microcirculation. The factors may be specifically constituent factors of blood that can practically reflect the flow rate or flow amount through the capillaries of the subject, more specifically one or more selected from a group consisting of leukocytes, erythrocytes, blood platelets and lymphocytes, although not being limited thereto. In addition, the target factors in the first blood stream may be labeled to obtain motion images. Specifically, the labeling may be achieved with one or more selected from a group consisting of a fluorescent dye, a transgenic probe and an antibody label. More specifically, the transgenic probe may be one or more selected from a group consisting of CFP (cyan fluorescent protein), YFP (yellow fluorescent protein), GFP (green fluorescent protein) and RFP (red fluorescent protein), although not being limited thereto. More specifically, the antibody label may be one conjugated with a fluorescent probe, e.g., an antibody conjugated with one or more fluorescent probe selected from a group consisting of Alexa 405, Alexa 488, Alexa 555 and Alexa 647, although not being limited thereto. In an example of the present disclosure, for the cease where the target factors in the first blood stream are erythrocytes, microcirculation was quantified by fluorescence-staining the erythrocytes with Vybrant DiD (V22887, ThermoFisher Scientific) and measuring the area traveled by the erythrocytes (functional capillary area) from a plurality of motion images thereof.

The method for quantitation of microcirculation in a subject may include a step of measuring the functional capillary area in which the target factors move in the first blood stream from the plurality of motion images.

The functional capillary refers to a capillary where the function of the capillary, e.g., exchange of oxygen, carbon dioxide, nutrients and other substances between blood and tissue through diffusion, occurs actively. The functional capillary may be a capillary through which the target factors in a first blood stream such as leukocytes, erythrocytes, blood platelets, lymphocytes, etc. pass or a capillary through which the target factors in a second blood stream such as neutrophils, etc. pass. A higher ratio of the functional capillary to the total capillary means that microcirculation in the subject is smooth or there is no microcirculatory disorder.

The functional capillary area may be measured by identifying the same target factors from the plurality of motion images, and may be calculated by measuring the area traveled by the target factors in the first blood stream from the change in their location over time. Specifically, the flow path of the target factors in the first blood stream passing through the capillaries of the subject over time may be measured from the plurality of motion images, and the functional capillary area may be measured from the flow path of the target factors in the first blood stream. More specifically, after measuring the flow path of the target factors in the first blood stream by comparing the plurality of images imaged with a time interval (t), the functional capillary area may be measured from the flow path of the target factors in the first blood stream.

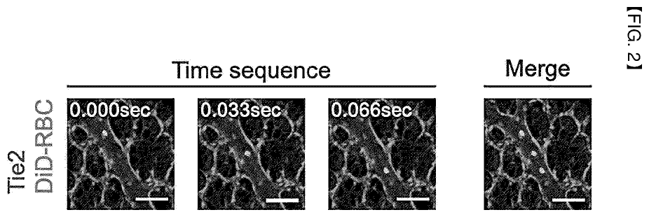

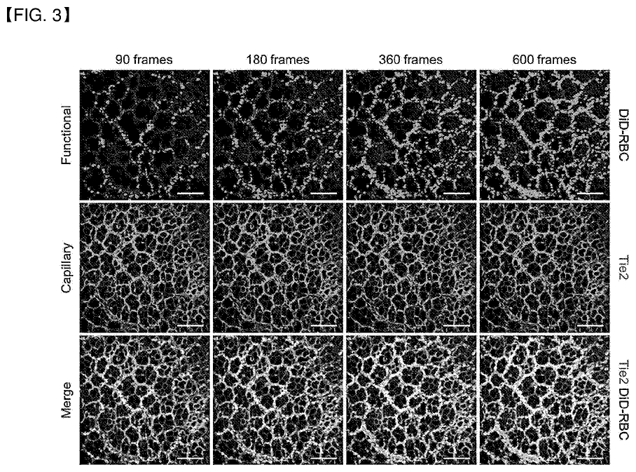

In an example of the present disclosure, the flow path of erythrocytes as target factors in a first blood stream passing through capillaries could be measured by fluorescence-staining the erythrocytes, acquiring the motion images of the erythrocytes with different time intervals (0.000 second, 0.033 second and 0.066 second) and merging the plurality of motion images of the erythrocytes (Experimental Example 1-1 and FIG. 2). In addition, functional capillary area could be measured from the flow path of the plurality of erythrocytes (Experimental Example 2 and FIG. 3).

The method for quantitation of microcirculation in a subject may include a step of calculating a functional capillary ratio (FCR) according to Formula 1.

Functional capillary ratio=functional capillary area/total capillary area. [Formula 1]

The quantitation of microcirculation may be accomplished by the calculation of the functional capillary ratio according to Formula 1. Because microcirculation can be quantified based on area rather than density, the area where through which one red blood cell passes and the area where through which a plurality of erythrocytes pass can be distinguished. Through this, microcirculation can be quantified more easily, conveniently and accurately because the space (area) through which erythrocytes pass actually can be reflected, and it is possible to quantify the microcirculation network which is difficult to quantify based on density.

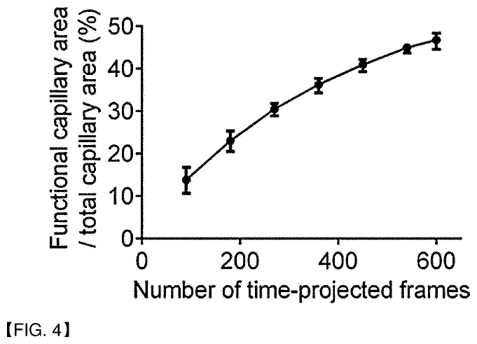

In an example of the present disclosure, the functional capillary ratio was calculated according to Formula 1 from the area traveled by DiD-stained erythrocytes as the functional capillary area and the vessel area detected by Tie2 or dextran signaling as the total capillary area. Through this, the microcirculation of the subject could be quantified (Experimental Example 2 and FIGS. 3 and 4).

In another aspect, the present disclosure provides an apparatus for measuring microcirculation in a subject, which acquires quantitative data on the microcirculation in the subject based on a plurality of motion images of target factors over time in a first blood stream passing through the capillaries of the subject according to Formula 1. Specifically, the apparatus may include: an imaging unit imaging target factors in a first blood stream passing through the capillaries of the subject; and a measuring unit acquiring quantitative data on the microcirculation in the subject according to Formula 1 based on the images imaged by the imaging unit. The subject, the microcirculation, the target factors in the first blood stream, the images and the quantitation of microcirculation are the same as described above.

Functional capillary ratio=functional capillary area/total capillary area. [Formula 1]

The measurement of microcirculation in the subject may be accomplished by measurement of quantitative data about the microcirculation in the subject, and the quantitative data about the microcirculation may be acquired by calculating the functional capillary ratio according to Formula 1.

The imaging unit may image a plurality of motion images of the target factors in the blood stream passing through the capillaries over time. For example, when the plurality of images are an image (M) at one time point (T) and images (M−1, M+1) at time points (T−1, T+1) before and after the time point (T) of the same time interval (t), because the three images (M−1, M, M+1) are respectively images at first time point (T−1), second time point (T) and third time point (T+1) with the same time interval (t), a flow path of target factors in a first blood stream or a second blood stream passing through the capillaries of the subject may occur over time in the three images (M−1, M, M+1). Therefore, quantitative data about microcirculation can be acquired by measuring the functional capillary area in which the target factor pass in the first blood stream. Specifically, the functional capillary area may be measured by identifying the same target factors in the three images (M−1, M, M+1) imaged with the time interval (t).

The time interval (t) of the three images (M−1, M, M+1) may be from 1/900 second to 1 second, specifically from 1/300 second to ⅓ second, more specifically 1/900 second or longer, 1/800 second or longer, 1/700 second or longer, 1/600 second or longer, 1/500 second or longer, 1/400 second or longer, 1/300 second or longer, 1/200 second or longer, 1/100 second or longer, 1/90 second or longer, 1/80 second or longer, 1/70 second or longer, 1/60 second or longer, 1/50 second or longer, 1/45 second or longer, 1/40 second or longer, 1/35 second or longer, 1/30 second or longer, 1/25 second or longer, 1/20 second or longer, 1/15 second or longer, 1/10 second or longer or ⅕ second or longer, and 1 second or shorter, ⅕ second or shorter, 1/10 second or shorter, 1/15 second or shorter, 1/20 second or shorter, 1/25 second or shorter, 1/30 second or shorter, 1/35 second or shorter, 1/40 second or shorter, 1/45 second or shorter, 1/50 second or shorter, 1/60 second or shorter, 1/70 second or shorter, 1/80 second or shorter, 1/90 second or shorter, 1/100 second or shorter, 1/200 second or shorter, 1/300 second or shorter, 1/400 second or shorter, 1/500 second or shorter, 1/600 second or shorter, 1/700 second or shorter, 1/800 second or shorter or 1/900 second or shorter. However, the time interval is not specially limited as long as microcirculation can be quantified from a plurality of motion images of the target factors in the first blood stream.

The plurality of motion images may be a plurality of images imaged at a frame rate or speed of 1-900 frames/second. The frame rate or speed may be specifically 3-300 frames/second, more specifically 1 frame/second or higher, 5 frames/second or higher, 10 frames/second or higher, 15 frames/second or higher, 20 frames/second or higher, 25 frames/second or higher, 30 frames/second or higher, 35 frames/second or higher, 40 frames/second or higher, 45 frames/second or higher, 50 frames/second or higher, 60 frames/second or higher, 70 frames/second or higher, 80 frames/second or higher, 90 frames/second or higher, 100 frames/second or higher, 200 frames/second or higher, 300 frames/second or higher, 400 frames/second or higher, 500 frames/second or higher, 600 frames/second or higher, 700 frames/second or higher or 800 frames/second or higher, and 900 frames/second or lower, 800 frames/second or lower, 700 frames/second or lower, 600 frames/second or lower, 500 frames/second or lower, 400 frames/second or lower, 300 frames/second or lower, 200 frames/second or lower, 100 frames/second or lower, 90 frames/second or lower, 80 frames/second or lower, 70 frames/second or lower, 60 frames/second or lower, 50 frames/second or lower, 45 frames/second or lower, 40 frames/second or lower, 35 frames/second or lower, 30 frames/second or lower, 25 frames/second or lower, 20 frames/second or lower, 15 frames/second or lower, 10 frames/second or lower or 5 frames/second or lower. However, the frame rate or speed is not specially limited as long as microcirculation can be quantified from a plurality of motion images of the target factors in the first blood stream, or the dynamic elements of the target factor can be analyzed from the plurality of motion images of the target factors in the second blood stream.

The imaging unit may be a confocal scanning laser microscope, a fluorescence microscope, a two-photon microscope or a three-photon microscope, although not being limited thereto.

The measuring unit may measure functional capillary area from the change in the location of the target factors in the first blood stream over time interval by identifying the same target factors from the plurality of motion images of the target factors in the first blood stream imaged by the imaging unit and may calculate the functional capillary ratio according to Formula 1. Specifically, after measuring the flow path of the target factors in the first blood stream passing through the capillaries of the subject over time from the plurality of motion images of the target factors in the first blood stream, the functional capillary area may be determined form the path of the target factors in the first blood stream. More specifically, by comparing the plurality of images imaged with the time interval (t), the flow path of the target factors in the first blood stream may be easily identified and traced, and the functional capillary ratio may be calculated according to Formula 1 by measuring the functional capillary area form the path of the target factors in the first blood stream. For measurement of the area depending on the flow path, the area may be determined through pixel analysis of the flow path. In an example of the present disclosure, an analysis program such as ImageJ was used.

In another aspect, the present disclosure provides a method for providing information for diagnosis of microcirculatory disorder in a subject, which includes a step of acquiring information for diagnosing microcirculatory disorder in the subject from the functional capillary ratio (FCR) calculated by the method for quantitation of microcirculation in a subject described above. The method for quantitation of microcirculation in a subject, the functional capillary ratio and the microcirculation are the same as described above.

In another aspect, the present disclosure provides an apparatus for providing information for diagnosis of microcirculatory disorder in a subject, which acquires information for diagnosing microcirculatory disorder in the subject from the calculated functional capillary ratio (FCR). The apparatus may include: a microcirculation quantitation unit acquiring quantitative data about the microcirculation of the subject according to Formula 1 based on the plurality of motion images of target factors in the first blood stream passing through the capillaries of the subject over time; and a microcirculatory disorder identification unit identifying microcirculatory disorder based on the acquired functional capillary ratio (FCR).

The microcirculatory disorder refers to abnormal microcirculation in which the target factors in the first blood stream such as leukocytes, erythrocytes, blood platelets, lymphocytes, etc. cannot smoothly pass through the capillaries. Specifically, the microcirculatory disorder may refer to a state where the functional capillary ratio is 70% or lower, 65% or lower, 60% or lower, 55% or lower, 50% or lower, 45% or lower, 40% or lower, 35% or lower, 30% or lower, 25% or lower, 20% or lower, 15% or lower, 10% or lower or 5% or lower as compared to the functional capillary ratio of a normal group with no microcirculatory disorder, or the functional capillary ratio is 0.4 or lower, 0.38 or lower, 0.36 or lower, 0.34 or lower, 0.32 or lower, 0.3 or lower, 0.28 or lower, 0.26 or lower, 0.24 or lower, 0.22 or lower, 0.2 or lower, 0.18 or lower, 0.16 or lower, 0.14 or lower, 0.12 or lower, 0.1 or lower, 0.08 or lower, 0.06 or lower, 0.04 or lower or 0.02 or lower. However, the range of the functional capillary ratio for identification of microcirculatory disorder may vary depending on the organ of the subject in which the capillaries are distributed, and is not limited to the above ranges.

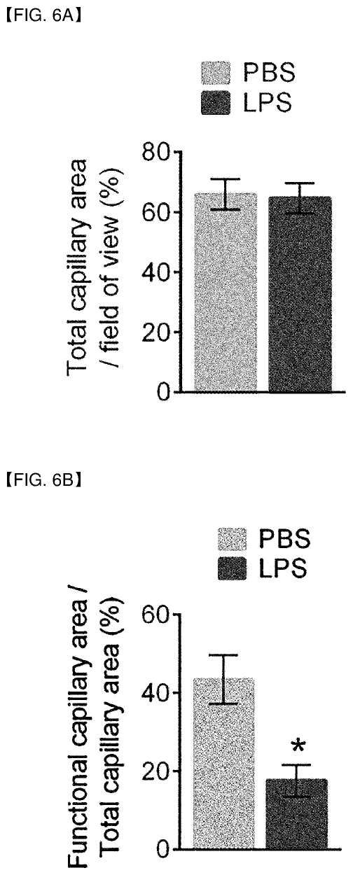

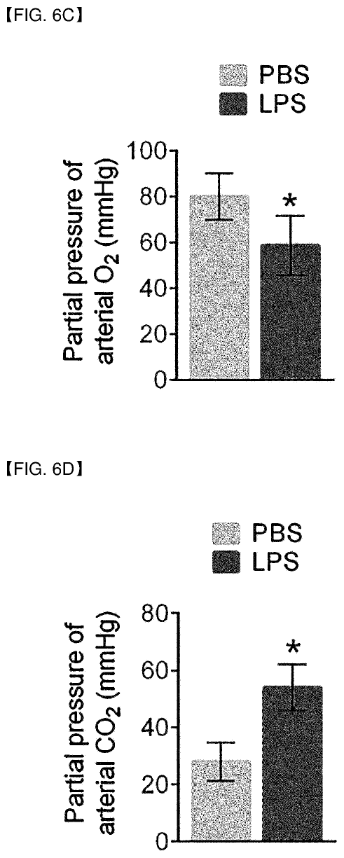

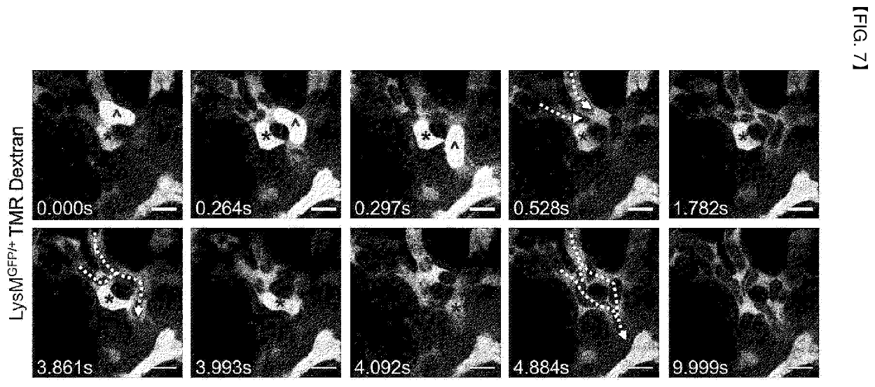

In an example of the present disclosure, a control group model (treated with PBS) and a sepsis-induced acute lung injury mouse model treated with LPS showed no difference in total capillary area. However, the acute lung injury mouse model had a functional capillary ratio (FCR) decreased by 50% or more as compared to the control group model as the functional capillary area in which erythrocytes pass was decreased rapidly (Experimental Example 3, FIG. 6A and FIG. 6B). Therefore, it can be seen that the microcirculatory disorder of a subject can be diagnosed easily and conveniently by measuring the functional capillary ratio according to the method of the present disclosure.

In another aspect, the present disclosure provides a method for providing information for diagnosis of microcirculatory disorder, which includes: a step of obtaining a plurality of motion images of target factors over time in a second blood stream flowing through the capillaries of the subject; a step of analyzing one or more dynamic element selected from a group consisting of sequestration time, track displacement length, track length, track velocity and track meandering index of the target factors in the second blood stream from the plurality of motion images; and a step of acquiring information for diagnosis of microcirculatory disorder in the subject from the dynamic element analysis result. Previously, endothelial dysfunction and vasoconstriction were suggested as potential central mechanisms in impaired systemic microcirculation, and it was known that sequestration in pulmonary capillaries functions as an immune surveillance system for detecting pathogens in the circulation. However, they could not explain how neutrophil sequestration progresses to microcirculatory disorder, particularly pulmonary microcirculatory disorder such as acute respiratory failure syndrome. In contrast, according to an exemplary embodiment of the present disclosure, it can be seen that cluster formation of neutrophils recruited during the early stage of sepsis-induced acute lung injury plays a key role in pulmonary microcirculatory disorder. Specifically, the neutrophils form clusters and act as an obstacle in capillaries and arterioles, leading to the redistribution and obstruction of the pulmonary microcirculation.

The method for providing information for diagnosis of microcirculatory disorder may include a step of obtaining a plurality of motion images of the target factors in the second blood stream flowing through the capillaries of the subject over time.

The target factors in the second blood stream are factors that pass through the microcirculatory capillaries of the subject. Information for diagnosis of microcirculatory disorder may be provided by analyzing the dynamic elements of the target factors, e.g., the sequestration time, track displacement length, track length, track velocity or track meandering index of the target factors, from the plurality of motion images of the target factors in the second blood stream over time. The target factors in the second blood stream may be the factors that pass through the capillaries of the subject along the microcirculation. They may be specifically the constituent factors of blood that can practically reflect the flow rate or flow amount through the capillaries of the subject, more specifically neutrophils, although not being limited thereto. In addition, the target factors in the second blood stream may be labeled to obtain motion images. Specifically, when the target factors in the second blood stream are neutrophils, the neutrophils may be labeled with a fluorophore-conjugated nucleic acid (DNA or RNA) encoding a peptide expressed in the neutrophils. In addition, the neutrophils may be labeled with an antibody specific for the neutrophils, and the antibody may be conjugated with a fluorophore. The nucleic acid encoding a peptide expressed in the neutrophils may be specifically one or more selected from a group consisting of a nucleic acid encoding a lysine motif (LysM) domain, a nucleic acid encoding leukocyte 6G (Ly6G), a nucleic acid encoding cluster of differentiation molecule 11B (CD11b) and a nucleic acid encoding cluster of differentiation molecule 18 (CD18). However, any nucleic acid allowing the analysis of dynamic elements from images by labeling the neutrophils may be used without limitation. And, the antibody specific for the neutrophils may be an antibody specific for a peptide expressed in the neutrophils, specifically an antibody specific for one or more selected from a group consisting of a lysine motif (LysM) domain, leukocyte 6G (Ly6G), cluster of differentiation molecule 11B (CD11b) and cluster of differentiation molecule 18 (CD18). However, any antibody allowing the analysis of dynamic elements from images by labeling the neutrophils may be used without limitation. In addition, the fluorophore may be specifically a transgenic probe or a fluorescent probe. More specifically, the transgenic probe may be one or more selected from a group consisting of CFP (cyan fluorescent protein), YFP (yellow fluorescent protein), GFP (green fluorescent protein) and RFP (red fluorescent protein), and the fluorescent probe may be one or more selected from a group consisting of Alexa 405, Alexa 488, Alexa 555 and Alexa 647, although not being limited thereto.

According to an exemplary embodiment of the present disclosure, when the target factors in the second blood stream are neutrophils, information for diagnosis of microcirculatory disorder may be provided by injecting an anti-Ly6G+ monoclonal antibody (Clone 1A8, 551459, BD Biosciences) conjugated with a fluorophore Alexa Fluor 555 or 647 (A-20005/A-20006, ThermoFisher Scientific) into the subject and monitoring the motion of neutrophils labeled with the antibody by measuring fluorescence signals. In addition, as previously known, the neutrophil-induced obstruction of blood flow increases the mismatching area of ventilation and perfusion, thereby intensifying hypoxia due to sepsis-induced acute respiratory failure syndrome (ARDS). Compared to the previous intravital imaging studies on the adhesion of neutrophils in the pulmonary capillaries (Yang N, Liu Y Y, Pan C S, Sun K, Wei X H, Mao X W, Lin F, Li X J, Fan J Y, Han J Y. Pretreatment with andrographolide pills® attenuates lipopolysaccharide-induced pulmonary microcirculatory disorder and acute lung injury in rats. Microcirculation 2014: 21(8): 703-716; Gill S E, Rohan M, Mehta S. Role of pulmonary microvascular endothelial cell apoptosis in murine sepsis-induced lung injury in vivo. Respir Res 2015: 16: 109), the method for providing information for diagnosis of microcirculatory disorder according to an exemplary embodiment of the present disclosure shows how dead space with ventilation/perfusion mismatch is created in the microcirculation by neutrophils. Moreover, the information can be provided more conveniently and accurately by directly imaging dead space fraction, which has been estimated indirectly by the difference in the partial pressure of arterial versus exhaled carbon dioxide using volumetric capnography.

The method for providing information for diagnosis of microcirculatory disorder may include a step of analyzing one or more dynamic element selected from a group consisting of sequestration time, track displacement length, track length, track velocity and track meandering index of the target factors in the second blood stream from the plurality of motion images.

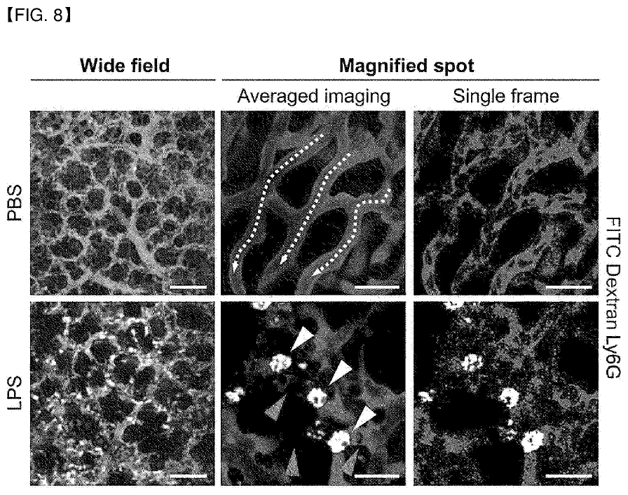

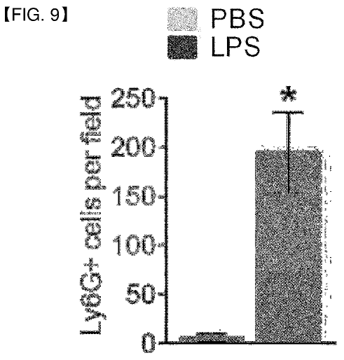

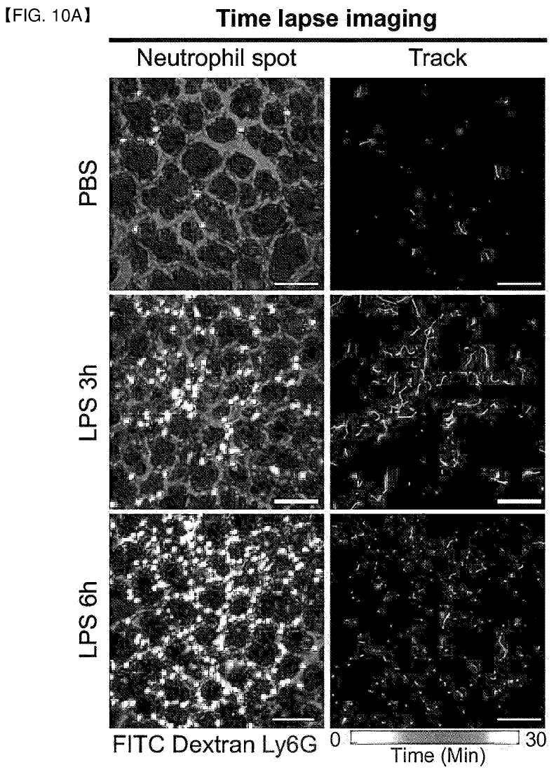

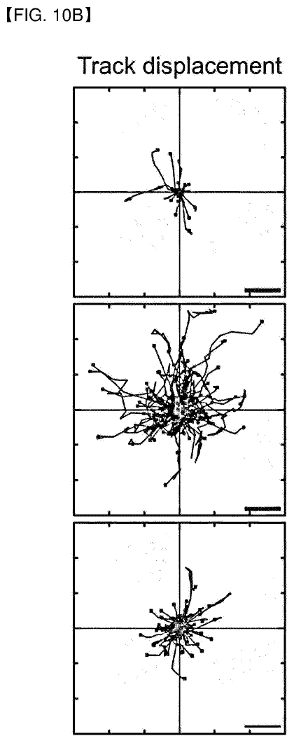

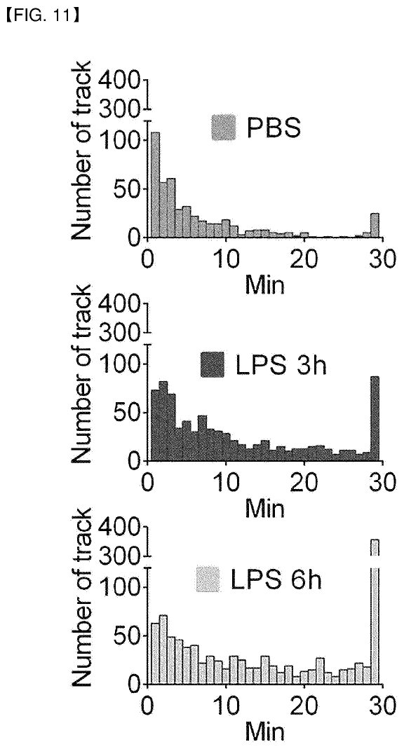

The sequestration time refers to the duration of time during which the target factors in the second blood stream are retained (sequestered) in a specific area of capillaries while passing through the capillaries. If the target factor in the second blood stream needs to be deformed to pass thorough the capillary because it has a large diameter, it requires longer time to pass thorough the capillary as compared to the blood flow rate. In this case, the target factor may be sequestered in a specific area of the capillary rather than passing through the capillary, leading to microcirculatory disorder. In an example of the present disclosure, whereas neutrophils passed through capillaries in a control mouse model (PBS), the flow of neutrophils was interrupted in numerous spots in a lung injury mouse model (ALI mouse model) (Experimental Example 4 and FIGS. 8 and 9). In another example of the present disclosure, it was confirmed that whereas most of neutrophils were sequestered very briefly for a control mouse model (PBS), the neutrophils of lung injury mouse models (LPS 3h and LPS 6h) were sequestered in specific area of capillaries due to lung injury and the proportion of sequestered neutrophils was dramatically increased as compared to the control group (Experimental Example 5, FIGS. 10A-10B and FIGS. 11A-11C).

The track displacement length refers to the amount of change of the location (unit: μm) of the target factors in the second blood stream over time. A larger value of the track displacement length means a higher motility of the target factors.

The track length refers to the distance (unit: μm) actually traveled by the target factors in the second blood stream over time. A larger value of the track length means a higher motility of the target factors.

The track velocity refers to the distance traveled the target factors in the second blood stream by per unit time (unit: μm/m). A larger value of the track velocity means a higher motility of the target factors.

The track meandering index refers to the tendency of the target factors in the second blood stream to move toward a target site or along a specific direction (unit: a.u., or arbitrary unit). A larger value of the track meandering index means a higher tendency of the target factors in the second blood stream to move toward a target site or along a specific direction and arrive at the target site within the fastest time. The track meandering index (a.u.) may be calculated using the Spots & Tracking function of the IMARIS program.

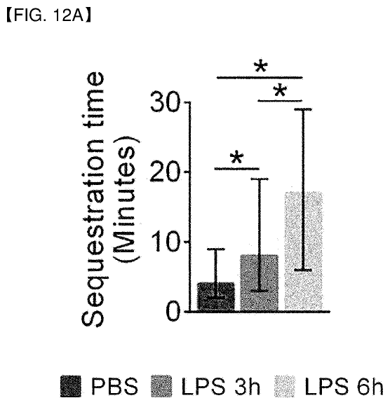

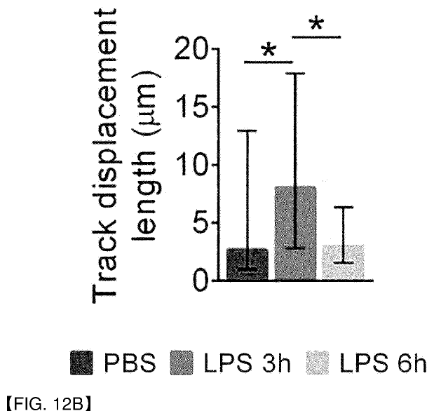

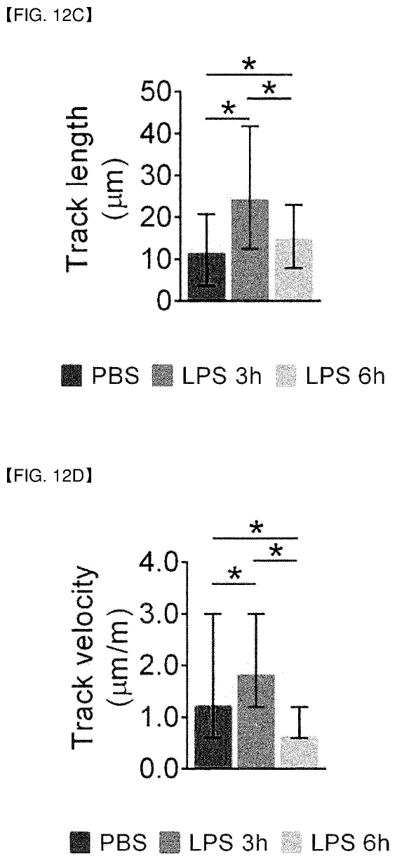

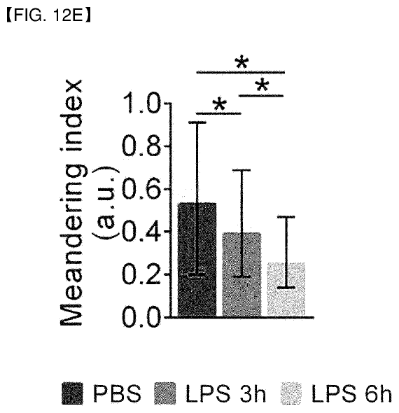

In an example of the present disclosure, lung injury mouse models (ALI mouse model, LPS 3h mouse model and LPS 6h mouse model) showed difference in the sequestration time, track displacement length, track length, track velocity and track meandering index when compared with a control group (PBS), suggesting that information for diagnosis of microcirculatory disorder in the subject can be provided through analysis of dynamic elements (Experimental Example 6 and FIGS. 12A-12E).

The analysis of dynamic elements may be accomplished by identifying the same target factors from the plurality of motion images. Specifically, the flow path of the target factors in the second blood stream passing through the capillaries of the subject over time may be measured from the plurality of motion images, and the functional capillary area may be measured from the flow path of the target factors in the second blood stream. More specifically, after measuring the flow path of the target factors in the second blood stream by comparing the plurality of images imaged with a time interval (t), dynamic elements may be measured from the flow path of the target factors in the second blood stream.

The method for providing information for diagnosis of microcirculatory disorder may include a step of acquiring information for diagnosis of microcirculatory disorder in the subject from the dynamic element analysis result.

If the sequestration time of the target factors in the second blood stream as the information for diagnosis of microcirculatory disorder in the subject is 5 minutes or longer, it may be diagnosed as microcirculatory disorder. As described above, when the subject has microcirculatory disorder, the sequestration time is increased as compared to a control group because the tendency of the target factors in the second blood stream remaining (entrapment) in specific area rather than passing through the capillaries is increased. Accordingly, if the sequestration time of the target factors in the second blood stream is 5 minutes or longer, it may be diagnosed as microcirculatory disorder. Specifically, if the sequestration time is 5 minutes or longer, 5 minutes and 10 seconds or longer, 5 minutes and 20 seconds or longer, 5 minutes and 30 seconds or longer, 5 minutes and 40 seconds or longer, 5 minutes and 50 seconds or longer, 6 minutes or longer, 6 minutes and 10 seconds or longer, 6 minutes and 20 seconds or longer, 6 minutes and 30 seconds or longer, 6 minutes and 40 seconds or longer, 6 minutes and 50 seconds or longer, 7 minutes or longer, 7 minutes and 10 seconds or longer, 7 minutes and 20 seconds or longer, 7 minutes and 30 seconds or longer, 7 minutes and 40 seconds or longer, 7 minutes and 50 seconds or longer, 8 minutes or longer, 8 minutes and 10 seconds or longer, 8 minutes and 20 seconds or longer, 8 minutes and 30 seconds or longer, 8 minutes and 40 seconds or longer, 8 minutes and 50 seconds or longer, 9 minutes or longer, 9 minutes and 10 seconds or longer, 9 minutes and 20 seconds or longer, 9 minutes and 30 seconds or longer, 9 minutes and 40 seconds or longer, 9 minutes and 50 seconds or longer, 10 minutes or longer, 11 minutes or longer, 12 minutes or longer, 13 minutes or longer, 14 minutes or longer, 15 minutes or longer, 16 minutes or longer, 17 minutes or longer, 18 minutes or longer or 19 minutes or longer, it may be diagnosed as microcirculatory disorder. However, because the range of the sequestration time for diagnosis of microcirculatory disorder may vary depending on the particular subject, the type of the capillary, the age, sex and body weight of the subject, the particular disease or pathological condition of the subject, or the severity of the disease or pathological condition and diagnosis of microcirculatory disorder based on these factors is within the level of those skilled in the art, the present disclosure is not limited to the above ranges. In an example of the present disclosure, neutrophil sequestration time was longer for lung injury mouse models (LPS administration groups) with about 8 minutes for a LPS 3h mouse model and about 18 minutes for a LPS 6h mouse model as compared to a control group (PBS, about 3 minutes). The sequestration time was about 2 times longer at 6 hours after the administration of LPS (LPS 6h mouse model) than at 3 hours after the administration of LPS (LPS 3h mouse model) (Experimental Example 6 and FIG. 12A).

If the track meandering index of the target factors in the second blood stream as the information for diagnosis of microcirculatory disorder in the subject is 0.4 a.u. or lower, it may be diagnosed as microcirculatory disorder. If the subject has microcirculatory disorder, the target factors in the second blood stream tend to be sequestered in specific area or move very slowly without directionality rather than passing through capillaries along the blood flow, when compared with a control group. Accordingly, it may be diagnosed as microcirculatory disorder if the track meandering index of the target factors in the second blood stream is 0.4 a.u. or lower. Specifically, it may be diagnosed as microcirculatory disorder if the track meandering index is 0.4 a.u. or lower, 0.39 a.u. or lower, 0.38 a.u. or lower, 0.37 a.u. or lower, 0.36 a.u. or lower, 0.35 a.u. or lower, 0.34 a.u. or lower, 0.33 a.u. or lower, 0.32 a.u. or lower, 0.31 a.u. or lower, 0.3 a.u. or lower, 0.29 a.u. or lower, 0.28 a.u. or lower, 0.27 a.u. or lower, 0.26 a.u. or lower, 0.25 a.u. or lower, 0.24 a.u. or lower, 0.23 a.u. or lower, 0.22 a.u. or lower, 0.21 a.u. or lower or 0.2 a.u. or lower. However, because the range of the track meandering index for diagnosis of microcirculatory disorder may vary depending on the particular subject, the type of the capillary, the age, sex and body weight of the subject, the particular disease or pathological condition of the subject, or the severity of the disease or pathological condition and diagnosis of microcirculatory disorder based on these factors is within the level of those skilled in the art, the present disclosure is not limited to the above ranges. In an example of the present disclosure, the track meandering index of neutrophils was lower lung injury mouse models (LPS administration groups) with about 0.4 a.u. for a LPS 3h mouse model and about 0.2 a.u. for a LPS 6h mouse model as compared to a control group (PBS, about 0.5 a.u.). The track meandering index was decreased to about ½ at 6 hours after the administration of LPS (LPS 6h mouse model) than at 3 hours after the administration of LPS (LPS 3h mouse model) (Experimental Example 6 and FIG. 12E).

When the dynamic element is one or more selected from a group consisting of track displacement length, track length and track velocity, a plurality of motion images of the target factors in the second blood stream over time may be a plurality of motion image sets imaged with a time interval (U) of 2 hours or longer as described below: (1) a plurality of first image sets (SET_M₁) at one time point (T₁), wherein the first image sets include an image (M₁) at the time point (T₁) and images (M₁−1, M₁+1) at time points (T₁−1, T₁+1) before and after the same time interval (t) with respect to the one time point (T₁);

(2) a plurality of second image sets (SET_M₂) at one time point (T₂) which is after the one time point (T₁) by a time (U) which is 2 hours or longer, wherein the second image sets include an image (M₂) at the time point (T₂) and images (M₂−1, M₂₊₁) at time points (T₂−1, T₂₊₁) before and after the same time interval (t) with respect to the time point (T₂); and

(3) a plurality of third image sets (SET_M₃) at a time point (T₃) which is after the one time point (T₂) by a time (U) which is 2 hours or longer, wherein the third image sets include an image (M₃) at the time point (T₃) and image (M₃−1, M₃₊₁) at time points (T₃-1, T₃₊₁) before and after the same time interval (t) with respect to the one time point (T₃).

The plurality of motion image sets may be obtained as described above in (1)-(3), and the plurality of motion image sets may be 2 or more, 3 or more, 4 or more, 5 or more or 6 or more image sets.

The time interval (U) between the plurality of image sets may be 2 hours or longer, 3 hours or longer, 4 hours or longer, 5 hours or longer or 6 hours or longer. However, the time interval is not limited to the above ranges as long as information for diagnosis of microcirculatory disorder can be provided by analyzing one or more dynamic element selected from a group consisting of the track displacement length, track length and track velocity of the target factors in the second blood stream from the plurality of image sets.

When the dynamic element is one or more selected from a group consisting of track displacement length, track length and track velocity, the dynamic elements may be analyzed sequentially in time from the plurality of motion image sets, and, if the dynamic element is decreased with time as a result of the analysis of the dynamic elements, it may be diagnosed as microcirculatory disorder. For a subject with microcirculatory disorder, e.g., if lung injury occurs due to endotoxin, the motility of neutrophils is increased during the early stage of lung injury but, as inflammation is aggravated due to microcirculatory disorder, the motility of neutrophils is decreased, thereby resulting in decreased track displacement length, track length and track velocity, as compared to a control group. Accordingly, if the track displacement length, track length or track velocity of the target factors in the second blood stream is decreased over time, it may be diagnosed as microcirculatory disorder. In an example of the present disclosure, the track displacement length, track length and track velocity were increased in a lung injury mouse model 3 hours after LPS administration (LPS 3h mouse model), when compared with a control group (PBS), and then were decreased to a level similar to that of the control group in a lung injury mouse model 6 hours after LPS administration (LPS 6h mouse model) (Experimental Example 6 and FIGS. 12B-12D).

The method for providing information for diagnosis of microcirculatory disorder may further include a step of detecting the generation of reactive oxygen species in the target factors in the second blood stream passing through the capillaries. Specifically, the detection of the generation of reactive oxygen species may be achieved by dihydroethidium (DHE) staining. However, any method capable of detecting the generation of reactive oxygen species in vivo or in situ may be used without limitation. If reactive oxygen species are generated in the target factors in the second blood stream, it may be diagnosed as microcirculatory disorder.

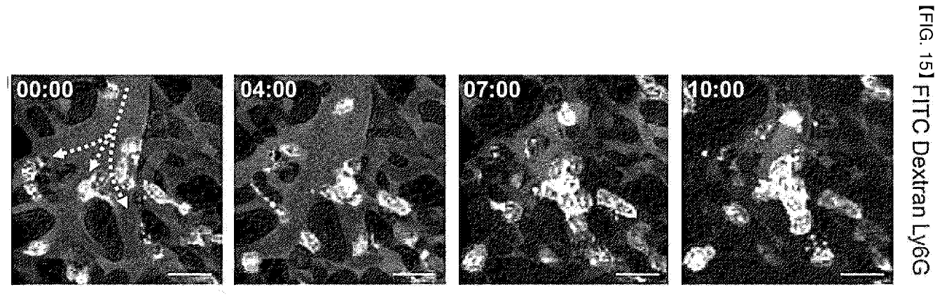

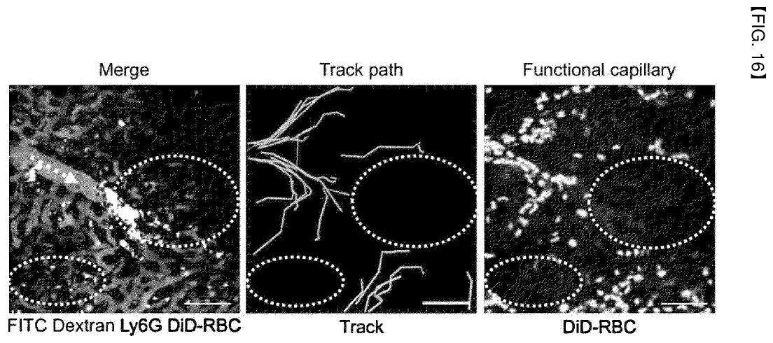

In an example of the present disclosure, whereas reactive oxygen species were not generated in the temporarily sequestered neutrophils of a control group (PBS), reactive oxygen species were generated in the neutrophils in capillaries of a lung injury mouse model (ALI mouse model), and the proportion of reactive oxygen species-generating neutrophils with respect to the total neutrophils was remarkably increased (Experimental Example 8 and FIGS. 16, 17A and 17B).

In another aspect, the present disclosure provides an apparatus for diagnosis of microcirculatory disorder, which includes: an imaging unit imaging target factors in a second blood stream passing through the capillaries of a subject; and an analysis unit analyzing one or more dynamic element selected from a group consisting of sequestration time, track displacement length, track length, track velocity and track meandering index of the target factors in the second blood stream based on the plurality of motion images imaged by the imaging unit. The subject, microcirculation, the microcirculatory disorder, the target factors in the second blood stream, the plurality of motion images, the dynamic elements, the analysis of the dynamic elements and the information for diagnosis of microcirculatory disorder are the same as described above.

The information for diagnosis of microcirculatory disorder may be acquired from the dynamic element analysis result of the target factors in the second blood stream of the subject.

The imaging unit may image a plurality of motion images of the target factors in the second blood stream passing through the capillaries over time. For example, when the plurality of images are an image (M) at one time point (T) and images (M−1, M+1) at time points (T−1, T+1) before and after the time point (T) of the same time interval (t), because the three images (M−1, M, M+1) are respectively images at second time point (T−1), second time point (T) and third time point (T+1) with the same time interval (t), a flow path of target factors in a first blood stream or a second blood stream passing through the capillaries of the subject may occur over time in the three images (M−1, M, M+1).

The time interval (t) of the three images (M−1, M, M+1) may be from 1/900 second to 1 second, specifically from 1/300 second to ⅓ second, more specifically 1/900 second or longer, 1/800 second or longer, 1/700 second or longer, 1/600 second or longer, 1/500 second or longer, 1/400 second or longer, 1/300 second or longer, 1/200 second or longer, 1/100 second or longer, 1/90 second or longer, 1/80 second or longer, 1/70 second or longer, 1/60 second or longer, 1/50 second or longer, 1/45 second or longer, 1/40 second or longer, 1/35 second or longer, 1/30 second or longer, 1/25 second or longer, 1/20 second or longer, 1/15 second or longer, 1/10 second or longer or ⅕ second or longer, and 1 second or shorter, ⅕ second or shorter, 1/10 second or shorter, 1/15 second or shorter, 1/20 second or shorter, 1/25 second or shorter, 1/30 second or shorter, 1/35 second or shorter, 1/40 second or shorter, 1/45 second or shorter, 1/50 second or shorter, 1/60 second or shorter, 1/70 second or shorter, 1/80 second or shorter, 1/90 second or shorter, 1/100 second or shorter, 1/200 second or shorter, 1/300 second or shorter, 1/400 second or shorter, 1/500 second or shorter, 1/600 second or shorter, 1/700 second or shorter, 1/800 second or shorter or 1/900 second or shorter. However, the time interval is not specially limited as long as microcirculation can be quantified from a plurality of motion images of the target factors in the second blood stream.

The plurality of motion images may be a plurality of images imaged at a frame rate or speed of 1-900 frames/second. The frame rate or speed may be specifically 3-300 frames/second, more specifically 1 frame/second or higher, 5 frames/second or higher, 10 frames/second or higher, 15 frames/second or higher, 20 frames/second or higher, 25 frames/second or higher, 30 frames/second or higher, 35 frames/second or higher, 40 frames/second or higher, 45 frames/second or higher, 50 frames/second or higher, 60 frames/second or higher, 70 frames/second or higher, 80 frames/second or higher, 90 frames/second or higher, 100 frames/second or higher, 200 frames/second or higher, 300 frames/second or higher, 400 frames/second or higher, 500 frames/second or higher, 600 frames/second or higher, 700 frames/second or higher or 800 frames/second or higher, and 900 frames/second or lower, 800 frames/second or lower, 700 frames/second or lower, 600 frames/second or lower, 500 frames/second or lower, 400 frames/second or lower, 300 frames/second or lower, 200 frames/second or lower, 100 frames/second or lower, 90 frames/second or lower, 80 frames/second or lower, 70 frames/second or lower, 60 frames/second or lower, 50 frames/second or lower, 45 frames/second or lower, 40 frames/second or lower, 35 frames/second or lower, 30 frames/second or lower, 25 frames/second or lower, 20 frames/second or lower, 15 frames/second or lower, 10 frames/second or lower or 5 frames/second or lower. However, the frame rate or speed is not specially limited as long as microcirculation can be quantified from a plurality of motion images of the target factors in the first blood stream, or the dynamic elements of the target factor can be analyzed from the plurality of motion images of the target factors in the second blood stream.

The imaging unit may be a confocal scanning laser microscope, a fluorescence microscope, a two-photon microscope or a three-photon microscope, although not being limited thereto.

The plurality of motion images imaged by the imaging unit may be a plurality of motion image sets imaged with a time interval (U) of 2 hours or longer as described below:

(1) a plurality of first image sets (SET_M₁) at one time point (T₁), wherein the first image sets include an image (M₁) at the time point (T₁) and images (M₁−1, M₁+1) at time points (T₁−1, T₁+1) before and after the same time interval (t) with respect to the one time point (T₁);

(2) a plurality of second image sets (SET_M₂) at one time point (T₂) which is after the one time point (T₁) by a time (U) which is 2 hours or longer, wherein the second image sets include an image (M₂) at the time point (T₂) and images (M₂−1, M₂₊₁) at time points (T₂−1, T₂₊₁) before and after the same time interval (t) with respect to the time point (T₂); and

(3) a plurality of third image sets (SET_M₃) at a time point (T₃) which is after the one time point (T₂) by a time (U) which is 2 hours or longer, wherein the third image sets include an image (M₃) at the time point (T₃) and image (M₃−1, M₃₊₁) at time points (T₃−1, T₃₊₁) before and after the same time interval (t) with respect to the one time point (T₃).

The plurality of motion image sets may be obtained as described above in (1)-(3), and the plurality of motion image sets may be 2 or more, 3 or more, 4 or more, 5 or more or 6 or more image sets.

The time interval (U) between the plurality of image sets may be 2 hours or longer, 3 hours or longer, 4 hours or longer, 5 hours or longer or 6 hours or longer. However, the time interval is not limited to the above ranges as long as information for diagnosis of microcirculatory disorder can be provided by analyzing one or more dynamic element selected from a group consisting of the track displacement length, track length and track velocity of the target factors in the second blood stream from the plurality of image sets.

The apparatus for diagnosis of microcirculatory disorder may further include a reactive oxygen species detection unit detecting the generation of reactive oxygen species in the target factors in the second blood stream. Specifically, the detection unit may detect the generation of reactive oxygen species by dihydroethidium (DHE) staining. However, any method capable of detecting the generation of reactive oxygen species in vivo or in situ may be used without limitation.

In another aspect, the present disclosure provides a composition for preventing, alleviating or treating lung injury, which contains an inhibitor against the expression or activity of macrophage-1 antigen (Mac-1) in neutrophils within pulmonary capillaries as an active ingredient, and prevents, alleviates or treats lung injury through alleviation of microcirculatory disorder in the lung. In addition, the present disclosure provides a composition for alleviating microcirculatory disorder in the lung, which contains an inhibitor against the expression or activity of macrophage-1 antigen (Mac-1) in neutrophils within pulmonary capillaries as an active ingredient.

In an aspect of the present disclosure, a subject which is a target for the prevention or treatment may be any subject requiring prevention or treatment of lung injury without limitation. Specifically, the subject may be a non-human animal such as monkey, dog, cat, rabbit, guinea pig, rat, mouse, cow, sheep, pig, goat, etc. or human, although not being limited thereto. In addition, the subject may be a subject having microcirculatory disorder, capillary circulatory disorder or peripheral circulatory disorder, although not being limited thereto.

The neutrophils are a type of granular leukocytes (granulocytes) produced mainly in the bone marrow. They account for 50-70% of leukocytes and about 90% of granulocytes in human blood, and are also called neutrocytes or heterophils. The neutrophils are leukocytes that arrive at the damaged or infected site first when tissue damage or microbial infection occurs. It is known that various chemotactic factors such as interleukin 8 (IL8), etc. are produced at the damaged or infected site and, as a result, neutrophils are recruited and cause acute inflammatory responses. The most distinguished functions of the neutrophils are phagocytosis and killing of bacteria. In an aspect of the present disclosure, the neutrophils may be neutrophils within pulmonary capillaries.

The macrophage-1 antigen (Mac-1) is an adhesion molecule belonging to the integrin family. It is a glycoprotein expressed in neutrophils, monocytes, macrophages, activated lymphocytes, etc., and is also called CD11b/CD18, which is a heterodimer wherein CD11b (αM chain, molecular weight ˜170,000 Da) and CD18 (β2 chain, molecular weight ˜95,000 Da) are noncovalently bound. The macrophage-1 antigen is stored in secretory granules in cells and can be rapidly expressed on cell surface following cell activation. The αM chain has three binding sites for divalent metal ions. The adhesion of this molecule is dependent on the divalent metal ions, and its ligands are ICAM−1, iC3b, fibrinogen, blood coagulation factor X, LPS (lipopolysaccharide), etc. The adhesion mediated by the molecule is involved in the adhesion of leukocytes to vascular endothelial cells, infiltration into tissues, phagocytosis, etc., and the molecule is known to associate with cytoskeletal structures, protein kinases, etc. in the cytoplasm and to be involved in signaling in the respiratory burst (oxidative burst) of leukocytes, etc.

Although an example of the present disclosure implies that neutrophil depletion alleviates pulmonary microcirculatory disorder, the effect of treatment-induced neutrophil depletion in sepsis is unclear because the effects on bacterial clearance and the response to systemic inflammation are unclear. Accordingly, the inventors of the present disclosure evaluated the subpopulation of neutrophils that may ameliorate lung injury. Flow cytometry showed that Mac-1 (CD11b/CD18), which interacts with ICAM−1 in endothelial cells and various coagulation factor, was significantly upregulated in the sequestered neutrophils in the lung of a lung injury model.

In the present specification, the term “gene expression” is used in the broadest concept, including transcription, translation, post-translational modification, etc.

The composition according to an aspect of the present disclosure may contain an inhibitor against the expression or activity of macrophage-1 antigen in neutrophils within pulmonary capillaries as an active ingredient.

The inhibitor against the expression or activity of macrophage-1 antigen may be a substance that inhibits the translation of an mRNA which encodes macrophage-1 antigen. Specifically, it may be an oligonucleotide binding to at least a portion of an mRNA which encodes macrophage-1 antigen, and may be one or more of a siRNA, a shRNA and a miRNA. The inhibitor against the expression or activity of macrophage-1 antigen may be one or more of a siRNA, a shRNA and a miRNA, which induce RNA interference (RNAi), and may provide an effect of preventing or treating lung injury through the RNAi phenomenon of interfering with an mRNA encoding the macrophage-1 antigen in order to inhibit the mRNA expression of a gene encoding the macrophage-1 antigen. The miRNA is an endogenous small RNA existing in cells. It is derived from a DNA that does not synthesize proteins and is generated from a hairpin-shaped transcript. The miRNA binds to the complementary sequence of the 3′-UTR of a target mRNA to induce the inhibition of the translation or destabilization of the mRNA, ultimately serving as a repressor to inhibit protein synthesis of the target mRNA. It is known that one miRNA can target several mRNAs and an mRNA can also be regulated by several miRNAs. Other RNAs that induce RNAi include the short interfering RNA (siRNA), which is a short RNA of about 19-27 mer, and the shRNA, which has a short hairpin structure.

The inhibitor against the expression or activity of macrophage-1 antigen may include a peptide binding specifically to macrophage-1 antigen in neutrophils, specifically an antibody binding specifically to macrophage-1 antigen in neutrophils. Specifically, the antibody may be an antibody binding specifically to CD11b or CD18, more specifically an antibody binding specifically to CD11 b, represented by an amino acid sequence of SEQ ID NO 1 or 2, more specifically an antibody binding specifically to a peptide having 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher or 99% or higher identity to the amino acid sequence of SEQ ID NO 1 or 2, further more specifically BD Biosciences' BD Pharmingen™ (Catalog Number: BD 553307), although not being limited thereto.

```
Amino acid sequence of CD11b (integrin
alpha-M isoform 1 precursor)
[SEQ ID NO 1]
      1 malrvlllta ltlchgfnld tenamtfqen
          argfgqsvvq lqgsrvvvga pqeivaanqr
    61 gslyqcdyst gscepirlqv pveavnmslg
          lslaattspp qllacgptvh qtcsentyvk
  121 glcflfgsnl rqqpqkfpea lrgcpqedsd
          iaflidgsgs iiphdfrrmk efvstvmeql
  181 kksktlfslm qyseefrihf tfkefqnnpn
          prslvkpitq llgrthtatg irkvvrelfn
  241 itngarknaf kilvvitdge kfgdplgyed
          vipeadregv iryvigvgda frseksrqel
  301 ntiaskpprd hvfqvnnfea lktiqnqlre
          kifaiegtqt gssssfehem sqegfsaait
  361 sngpllstvg sydwaggvfl ytskekstfi
          nmtrvdsdmn daylgyaaai ilrnrvqslv
  421 lgapryqhig lvamfrqntg mwesnanvkg
          tqigayfgas lcsvdvdsng stdlvligap
  481 hyyeqtrggq vsvcplprgq rarwqcdavl
          ygeqgqpwgr fgaaltvlgd vngdkltdva
  541 igapgeednr gavylfhgts gsgispshsq
          riagsklspr lqyfgqslsg gqdltmdglv
  601 dltvgaqghv lllrsqpvlr vkaimefnpr
          evarnvfecn dqvvkgkeag evrvclhvqk
  661 strdrlregq iqsvvtydla ldsgrphsra
          vfnetknstr rqtqvlgltq tcetlklqlp
  721 nciedpvspi vlrlnfslvg tplsafgnlr
          pvlaedaqrl ftalfpfekn cgndnicqdd
  781 Isitfsfmsl dclvvggpre fnvtvtvrnd
          gedsyrtqvt fffpldlsyr kvstlqnqrs
  841 qrswrlaces asstevsgal kstscsinhp
          ifpensevtf nitfdvdska slgnklllka
  901 nvtsennmpr tnktefqlel pvkyavymvv
          tshgvstkyl nftasentsr vmqhqyqvsn
  961 Igqrslpisl vflvpvrlnq tviwdrpqvt
          fsenlsstch tkerlpshsd flaelrkapv
1021 vncsiavcqr iqcdipffgi qeefnatlkg
          nlsfdwyikt shnhllivst aeilfndsvf
1081 tllpgqgafv rsqtetkvep fevpnplpli
          vgssvgglll lalitaalyk Igffkrqykd
1141 mmseggppga epq
Amino acid sequence of CD11b
(integrin alpha-M isoform
precursor)
[SEQ ID NO 2]
      1 malrvlllta ltlchgfnld tenamtfqen
          argfgqsvvq lqgsrvvvga pqeivaanqr
    61 gslyqcdyst gscepirlqv pveavnmslg
          lslaattspp qllacgptvh qtcsentyvk
  121 glcflfgsnl rqqpqkfpea lrgcpqedsd
          iaflidgsgs iiphdfrrmk efvstvmeql
  181 kksktlfslm qyseefrihf tfkefqnnpn
          prslvkpitq llgrthtatg irkvvrelfn
  241 itngarknaf kilvvitdge kfgdplgyed
          vipeadregv iryvigvgda frseksrqel
  301 ntiaskpprd hvfqvnnfea lktiqnqlre
          kifaiegtqt gssssfehem sqegfsaait
  361 sngpllstvg sydwaggvfl ytskekstfi
          nmtrvdsdmn daylgyaaai ilrnrvqslv
  421 lgapryqhig lvamfrqntg mwesnanvkg
          tqigayfgas lcsvdvdsng stdlvligap
  481 hyyeqtrggq vsvcplprgr arwqcdavly
          geqgqpwgrf gaaltvlgdv ngdkltdvai
  541 gapgeednrg avylfhgtsg sgispshsqr
          iagsklsprl qyfgqslsgg qdltmdglvd
  601 ltvgaqghvl llrsqpvlrv kaimefnpre
          varnvfecnd qvvkgkeage vrvclhvqks
  661 trdrlregqi qsvvtydlal dsgrphsrav
          fnetknstrr qtqvlgltqt cetlklqlpn
  721 ciedpvspiv lrlnfslvgt plsafgnlrp
          vlaedaqrlf talfpfeknc gndnicqddl
  781 sitfsfmsld clvvggpref nvtvtvrndg
          edsyrtqvtf ffpldlsyrk vstlqnqrsq
  841 rswrlacesa sstevsgalk stscsinhpi
          fpensevtfn itfdvdskas lgnklllkan
  901 vtsennmprt nktefqlelp vkyavymvvt
          shgvstkyln ftasentsrv mqhqyqvsnl
  961 gqrslpislv flvpvrlnqt viwdrpqvtf
          senlsstcht kerlpshsdf laelrkapw
1021 ncsiavcqri qcdipffgiq eefnatlkgn
          lsfdwyikts hnhllivsta eilfndsvft
1081 llpgqgafvr sqtetkvepf evpnplpliv
          gssvggllll alitaalykl gffkrqykdm
1141 mseggppgae pq
```

Alternatively, the antibody may be specifically abciximab, and the abciximab may be ISU Abxis's abciximab (Clotinab), although not being limited thereto.

The inhibitor against the expression or activity of macrophage-1 antigen may be contained at a concentration of 0.2-20 mg/mL, specifically 0.2 mg/mL or higher, 0.3 mg/mL or higher, 0.4 mg/mL or higher, 0.5 mg/mL or higher, 0.6 mg/mL or higher, 0.7 mg/mL or higher, 0.8 mg/mL or higher, 0.9 mg/mL or higher, 1 mg/mL or higher, 1.1 mg/mL or higher, 1.2 mg/mL or higher, 1.3 mg/mL or higher, 1.4 mg/mL or higher, 1.5 mg/mL or higher, 1.6 mg/mL or higher, 1.7 mg/mL or higher, 1.8 mg/mL or higher, 1.9 mg/mL or higher, 2 mg/mL or higher, 3 mg/mL or higher, 4 mg/mL or higher, 5 mg/mL or higher, 6 mg/mL or higher, 7 mg/mL or higher, 8 mg/mL or higher, 9 mg/mL or higher, 10 mg/mL or higher, 15 mg/mL or higher or 20 mg/mL or higher and 20 mg/mL or lower, 15 mg/mL or lower, 10 mg/mL or lower, 9 mg/mL or lower, 8 mg/mL or lower, 7 mg/mL or lower, 6 mg/mL or lower, 5 mg/mL or lower, 4 mg/mL or lower, 3.9 mg/mL or lower, 3.8 mg/mL or lower, 3.7 mg/mL or lower, 3.6 mg/mL or lower, 3.5 mg/mL or lower, 3.4 mg/mL or lower, 3.3 mg/mL or lower, 3.2 mg/mL or lower, 3.1 mg/mL or lower, 3 mg/mL or lower, 2.9 mg/mL or lower, 2.8 mg/mL or lower, 2.7 mg/mL or lower, 2.6 mg/mL or lower, 2.5 mg/mL or lower, 2.4 mg/mL or lower, 2.3 mg/mL or lower, 2.2 mg/mL or lower, 2.1 mg/mL or lower, 2 mg/mL or lower, 1.5 mg/mL or lower, 1 mg/mL or lower, 0.5 mg/mL or lower or 0.2 mg/mL or lower, based on the total volume of the composition, although not being limited thereto.

The microcirculatory disorder refers to abnormal microcirculation in which leukocytes, erythrocytes, blood platelets, lymphocytes, etc. cannot smoothly pass through the capillaries. Specifically, the microcirculatory disorder may refer to a state where the functional capillary ratio (FCR) according to Formula 1 is 70% or lower, 65% or lower, 60% or lower, 55% or lower, 50% or lower, 45% or lower, 40% or lower, 35% or lower, 30% or lower, 25% or lower, 20% or lower, 15% or lower, 10% or lower or 5% or lower as compared to the functional capillary ratio of a normal group with no microcirculatory disorder, or the functional capillary ratio is 0.4 or lower, 0.38 or lower, 0.36 or lower, 0.34 or lower, 0.32 or lower, 0.3 or lower, 0.28 or lower, 0.26 or lower, 0.24 or lower, 0.22 or lower, 0.2 or lower, 0.18 or lower, 0.16 or lower, 0.14 or lower, 0.12 or lower, 0.1 or lower, 0.08 or lower, 0.06 or lower, 0.04 or lower or 0.02 or lower. However, the range of the functional capillary ratio for identification of microcirculatory disorder may vary depending on the organ of the subject in which the capillaries are distributed, and is not limited to the above ranges.

Functional capillary ratio=functional capillary area/total capillary area [Formula 1]

The alleviation of microcirculatory disorder may be increasing a functional capillary ratio, which is the ratio of the area of functional capillaries through which erythrocytes pass to the total capillary area of the lung. Specifically, it may be increasing the functional capillary ratio (FCR) according to Formula 1.

Functional capillary ratio=functional capillary area/total capillary area [Formula 1]

The functional capillary area may be measured by identifying the same target factors from a plurality of motion images of the target factors such as leukocytes, erythrocytes, blood platelets, lymphocytes, neutrophils, etc. in a first blood stream or a second blood stream, and may be calculated by measuring the area traveled by the target factors in the first blood stream from the change in their location over time. Specifically, the flow path of the target factors in the first blood stream or second blood stream passing through the capillaries of the subject over time may be measured from the plurality of motion images, and the functional capillary area may be measured from the flow path of the target factors in the first blood stream or second blood stream. More specifically, after measuring the flow path of the target factors in the first blood stream or second blood stream by comparing the plurality of images imaged with a time interval (t), the functional capillary area may be measured from the flow path of the target factors in the first blood stream or second blood stream. More specifically, after measuring the flow path of the target factors in the first blood stream or second blood stream by comparing the plurality of images imaged with a time interval (t), the functional capillary area may be measured from the flow path of the target factors in the first blood stream or second blood stream.

The lung injury may be a disease caused by microcirculatory disorder in the lung, specifically one or more disease selected from a group consisting of pulmonary vasoconstriction, asthma, respiratory failure, respiratory distress syndrome (RDS), acute respiratory failure syndrome (ARDS), cystic fibrosis (CF), allergic rhinitis (AR), pulmonary hypertension, emphysema, chronic obstructive pulmonary disease (COPD), pulmonary graft rejection, lung infection, bronchitis and cancer. However, the disease is not specially limited as long as it is a disease caused by microcirculatory disorder in the lung.

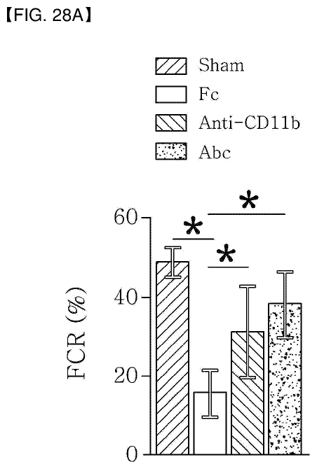

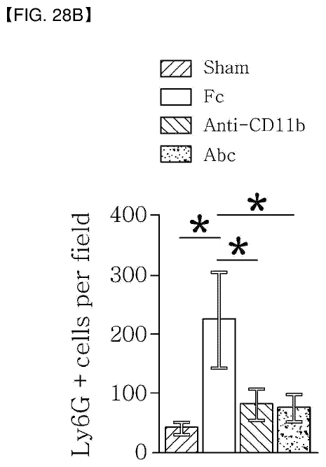

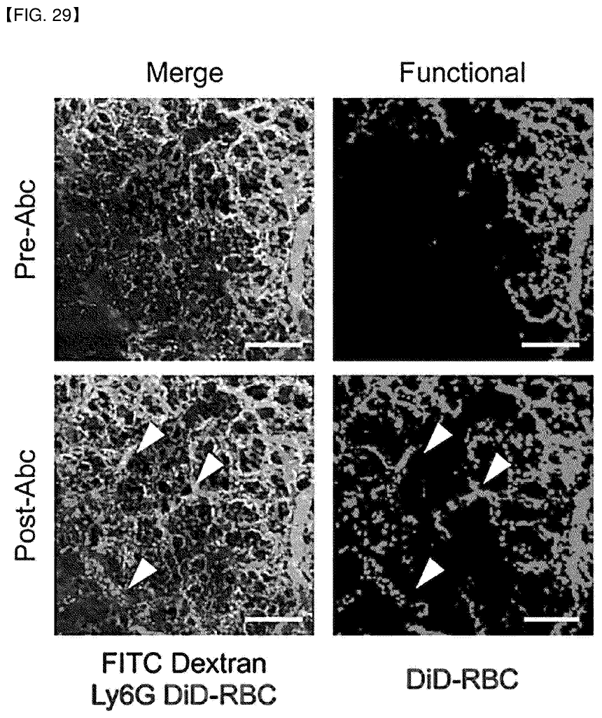

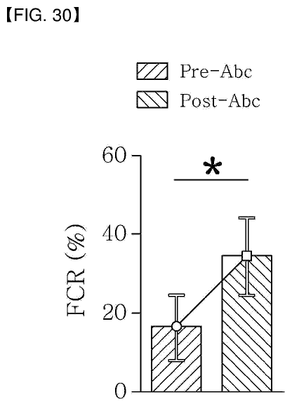

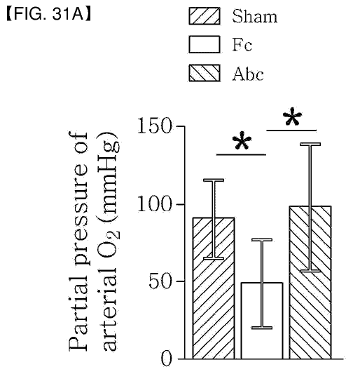

The inhibitor against the expression or activity of macrophage-1 antigen according to an aspect of the present disclosure may increase the functional capillary ratio (FCR) in the pulmonary capillaries of the subject as compared to a normal control group or a control group prior to the administration of the inhibitor. Specifically, it may increase the functional capillary ratio (FCR) by 1.1 times or more, 1.2 times or more, 1.3 times or more, 1.4 times or more, 1.5 times or more, 1.6 times or more, 1.7 times or more, 1.8 times or more, 1.9 times or more, 2 times or more, 2.1 times or more, 2.2 times or more, 2.3 times or more, 2.4 times or more, 2.5 times or more, 2.6 times or more, 2.7 times or more, 2.8 times or more, 2.9 times or more or 3 times or more. However, the range of the increase of the functional capillary ratio is not limited to the above ranges as long as the pulmonary microcirculatory disorder of the subject can be alleviated. In an example of the present disclosure, it was confirmed that the FCR was decreased by 50% or higher in a CLP mouse model (CLP mouse model or pre-Abc mouse model) having pulmonary microcirculatory disorder as compared to a normal group (Sham), and pulmonary microcirculatory disorder was alleviated as the FCR was increased by about 2 times or more when the expression or activity of Mac-1 was inhibited (anti-Mac-1 mouse model, abciximab mouse model or post-Abc mouse model), suggesting that the composition according to an exemplary embodiment of the present disclosure can prevent or treat lung injury (Experimental Example 12-2, FIG. 28A, Experimental Example 12-3 and FIG. 30).

The inhibitor against the expression or activity of macrophage-1 antigen according to an aspect of the present disclosure may decrease the number of sequestered neutrophils per unit area (512×512 μm) of pulmonary capillaries in a subject as compared to a normal control group or a control group prior to the administration of the inhibitor. Specifically, it may decrease the number of sequestered neutrophils per unit area (512×512 μm) of pulmonary capillaries by 10% or more, 15% or more, 20% or more, 25% or more, 30% or more, 35% or more, 40% or more, 45% or more, 50% or more or 55% or more as compared to a normal control group or a control group prior to the administration of the inhibitor. However, the range of the decrease of the number of sequestered neutrophils per unit area (512×512 μm) of pulmonary capillaries is not limited to the above ranges as long as the pulmonary microcirculatory disorder of the subject can be alleviated.

In this aspect, the composition may be a pharmaceutical composition or a food composition.

The pharmaceutical composition may be prepared into a solid, semisolid or liquid form for oral administration by adding a commonly used inorganic or organic carrier to the active ingredient.

Preparations for oral administration may include a tablet, a pill, a granule, a soft/hard capsule, a dust, a fine granule, a powder, an emulsion, a syrup, a pellet, etc. The active ingredient of the present disclosure may be prepared into a preparation form by a method commonly used in the art, and a surfactant, an excipient, a colorant, a fragrance, a preservative, a stabilizer, a buffer, a suspending agent or other adjuvants commonly used in the art may be used adequately.

The pharmaceutical composition according to the present disclosure may be usefully used for preventing or treating lung injury, specifically lung injury caused by pulmonary microcirculatory disorder, more specifically lung injury in which the number of sequestered neutrophils in pulmonary capillaries is increased, dead space is increased or the number of erythrocytes passing through capillaries is decreased due to pulmonary microcirculatory disorder. The lung injury may be pulmonary vasoconstriction, asthma, respiratory failure, respiratory distress syndrome (RDS), acute respiratory failure syndrome (ARDS), cystic fibrosis (CF), allergic rhinitis (allergic rhinitis, AR), pulmonary hypertension, emphysema, chronic obstructive pulmonary disease (COPD), pulmonary graft rejection, lung infection, bronchitis, cancer, etc., although not being limited thereto.

The pharmaceutical composition may be administered orally, rectally, topically, transdermally, intravenously, intramuscularly, intraperitoneally, subcutaneously, etc.

In addition, the administration dosage of the composition or the active ingredient in the composition will vary depending on the age, sex and body weight of a subject to be treated, the particular disease or pathological condition to be treated, the severity of the disease or pathological condition, administration route and the discretion of a diagnoser. Determination of the administration dosage based on these factors is within the level of those skilled in the art. The administration dosage may be 0.001-2000 mg/kg/day, specifically 0.5-1500 mg/kg/day, more specifically 0.001 mg/kg/day or more, 0.01 mg/kg/day or more, 0.1 mg/kg/day or more, 0.5 mg/kg/day or more, 1 mg/kg/day or more, 10 mg/kg/day or more, 50 mg/kg/day or more, 100 mg/kg/day or more, 150 mg/kg/day or more, 200 mg/kg/day or more, 250 mg/kg/day or more, 300 mg/kg/day or more, 350 mg/kg/day or more, 400 mg/kg/day or more, 450 mg/kg/day or more, 500 mg/kg/day or more, 550 mg/kg/day or more, 600 mg/kg/day or more, 650 mg/kg/day or more, 700 mg/kg/day or more, 750 mg/kg/day or more, 800 mg/kg/day or more, 850 mg/kg/day or more, 900 mg/kg/day or more, 950 mg/kg/day or more, 1000 mg/kg/day or more, 1050 mg/kg/day or more, 1100 mg/kg/day or more, 1150 mg/kg/day or more, 1200 mg/kg/day or more, 1250 mg/kg/day or more, 1300 mg/kg/day or more, 1350 mg/kg/day or more, 1400 mg/kg/day or more, 1450 mg/kg/day or more, 1500 mg/kg/day or more, 1550 mg/kg/day or more, 1600 mg/kg/day or more, 1650 mg/kg/day or more, 1700 mg/kg/day or more, 1750 mg/kg/day or more, 1800 mg/kg/day or more, 1850 mg/kg/day or more, 1900 mg/kg/day or more or 1950 mg/kg/day or more and 2000 mg/kg/day or less, 1950 mg/kg/day or less, 1900 mg/kg/day or less, 1850 mg/kg/day or less, 1800 mg/kg/day or less, 1750 mg/kg/day or less, 1700 mg/kg/day or less, 1650 mg/kg/day or less, 1600 mg/kg/day or less, 1550 mg/kg/day or less, 1500 mg/kg/day or less, 1450 mg/kg/day or less, 1400 mg/kg/day or less, 1350 mg/kg/day or less, 1300 mg/kg/day or less, 1250 mg/kg/day or less, 1200 mg/kg/day or less, 1150 mg/kg/day or less, 1100 mg/kg/day or less, 1050 mg/kg/day or less, 1000 mg/kg/day or less, 950 mg/kg/day or less, 900 mg/kg/day or less, 850 mg/kg/day or less, 800 mg/kg/day or less, 750 mg/kg/day or less, 700 mg/kg/day or less, 650 mg/kg/day or less, 600 mg/kg/day or less, 550 mg/kg/day or less, 500 mg/kg/day or less, 450 mg/kg/day or less, 400 mg/kg/day or less, 350 mg/kg/day or less, 300 mg/kg/day or less, 250 mg/kg/day or less, 200 mg/kg/day or less, 150 mg/kg/day or less, 100 mg/kg/day or less, 50 mg/kg/day or less, 10 mg/kg/day or less, 1 mg/kg/day or less, 0.5 mg/kg/day or less, 0.1 mg/kg/day or less or 0.01 mg/kg/day or less.

The food composition may be a health food composition, and may be prepared by adequately adding the inhibitor against the expression or activity of macrophage-1 antigen of the present disclosure to a food either alone or in combination with another food ingredient according to a common method.

The health food is not particularly limited. Examples of the food to which the active ingredient can be added are meat, sausage, bread, chocolate, candy, snack, confectionery, pizza, ramen, other noodles, gum, dairy products including ice cream, soups, beverages, tea, drinks, alcoholic beverages, vitamin complexes, etc., and include all health foods in the usual meaning.

A health beverage composition of the present disclosure may contain various flavorants, natural carbohydrates, etc. as additional ingredients like conventional beverages. The natural carbohydrate may be a monosaccharide such as glucose or fructose, a disaccharide such as maltose or sucrose, a polysaccharide such as dextrin or cyclodextrin, or a sugar alcohol such as xylitol, sorbitol, erythritol, etc. As a sweetener, a natural sweetener such as thaumatin and stevia extract or a synthetic sweetener such as saccharin and aspartame may be used. The natural carbohydrate may be contained in an amount of 0.01-0.04 wt %, specifically 0.02-0.03 wt %, based on the total weight of the composition of the present disclosure.

In addition, the health food of the present disclosure may contain various nutrients, vitamins, electrolytes, flavorants, colorants, pectic acid and its salts, alginic acid and its salts, organic acids, protective colloidal thickeners, pH adjusters, stabilizers, antiseptics, glycerin, alcohols, carbonating agents used in carbonated beverages, etc. In addition, it may contain a pulp for preparation of natural fruit juice, fruit juice beverages and vegetable beverages. These ingredients may be used independently or in combination. The proportion of these additives may be 0.01-0.1 wt % based on the total weight of the composition of the present disclosure.

In another aspect, the present disclosure provides a method for screening a substance for preventing, alleviating or treating lung injury, which includes: (a) a step of preparing a lung injury model; (b) a step of treating the lung injury model with a test substance; (c) a step of measuring the change in the expression or activity of macrophage-1 antigen (Mac-1) in neutrophils within pulmonary capillaries of the lung injury model caused by the test substance; and (d) a step of identifying whether the test substance increases a functional capillary ratio, which is the ratio of the area of functional capillaries through which erythrocytes pass to the total capillary area of the lung injury model. The microcirculation, the microcirculatory disorder, the neutrophils, the macrophage-1 antigen and the lung injury are the same as described above.

The lung injury model may be a non-human animal such as monkey, dog, cat, rabbit, guinea pig, rat, mouse, cow, sheep, pig, goat, etc., specifically a sepsis-induced lung-injured non-human animal, more specifically a non-human animal in which sepsis has been induced by LPS administration or a CLP (cecal ligation and puncture) non-human animal mode in which the appendix has been punctured and then ligated, although not being limited thereto.

In an exemplary embodiment of the present disclosure, “relative expression level” may refer to the degree of the inhibition of the expression or activity of macrophage-1 antigen in neutrophils within pulmonary capillaries after treatment with a test substance as compared to the expression or activity of macrophage-1 antigen in neutrophils within pulmonary capillaries before treatment with the test substance. Alternatively, the “relative expression level” may refer to the degree of the inhibition of the expression or activity of macrophage-1 antigen in neutrophils within pulmonary capillaries treated with the test substance as compared to the expression or activity of macrophage-1 antigen in neutrophils within pulmonary capillaries not treated with the test substance. For example, the relative expression level may include the relative expression level of an mRNA or the relative expression level of a protein.

In the step (c), the expression or activity of macrophage-1 antigen in neutrophils within pulmonary capillaries may be compared before and after treating the lung injury model with the test substance. Alternatively, in the step (c), the expression or activity of macrophage-1 antigen in neutrophils within pulmonary capillaries may be compared for a lung injury model treated with the test substance and a lung injury model not treated with the test substance.

In addition, the screening method may further include a step of, if the expression or activity of macrophage-1 antigen is decreased as compared to before the treatment with the test substance as a result of measuring the expression or activity in the step (c) or if a functional capillary ratio, which is the ratio of the area of functional capillaries through which erythrocytes pass to the total capillary area of the lung injury model, is increased, identifying the test substance as a substance for preventing, alleviating or treating lung injury.

In the step of identifying as a substance for preventing, alleviating or treating lung injury, the test substance may be identified as a substance for preventing, alleviating or treating lung injury if the expression or activity of macrophage-1 antigen has decreased by about 10% or more as a result of measuring the expression or activity in the step (c). That is to say, if the expression or activity has decreased by about 10% or more when the expression or activity of macrophage-1 antigen in neutrophils within pulmonary capillaries of the lung injury model treated with the test substance was compared with the expression or activity of macrophage-1 antigen in neutrophils within pulmonary capillaries of the lung injury model before treatment with the test substance, it may be identified as a substance for preventing, alleviating or treating lung injury. Alternatively, if the expression or activity has decreased by about 10% or more when the expression or activity of macrophage-1 antigen in neutrophils within pulmonary capillaries of the lung injury model treated with the test substance was compared with the expression or activity of macrophage-1 antigen in neutrophils within pulmonary capillaries of the lung injury model not treated with the test substance, it may be identified as a substance for preventing, alleviating or treating lung injury. For example, when the expression or activity of macrophage-1 antigen has decreased by 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more or 90% or more as compared to before the treatment with the test substance, it may be identified as a substance for preventing, alleviating or treating lung injury, although not being limited thereto. The degree of the expression or activity is measured within statistical significance. The statistical significance refers to significant difference achieved through biological statistical analysis, quantitatively with a p-value smaller than 0.05.

In an exemplary embodiment, the degree of the expression or activity of macrophage-1 antigen may be identified by known techniques, e.g., reverse transcription polymerase chain reaction (RT-PCR), ELISA, western blot or immune blot, although not being limited thereto.

In the step of identifying as a substance for preventing, alleviating or treating lung injury, the test substance may be identified as a substance for preventing, alleviating or treating lung injury if the functional capillary ratio, i.e. the ratio of the area passed by erythrocytes to the total capillary area, has increased by about 1.1 times or more in the step (c). Specifically, the test substance may be identified as a substance for preventing, alleviating or treating lung injury if the functional capillary ratio (FCR) according to Formula 1 of a lung injury model has increased by 1.1 times or more after treatment with the test substance as compared to before the treatment with the test substance. Alternatively, the test substance may be identified as a substance for preventing, alleviating or treating lung injury if the functional capillary ratio has increased by about 1.1 times or more in a lung injury model treated with the test substance as compared to a lung injury model not treated with the test substance.

Functional capillary ratio=functional capillary area/total capillary area [Formula 1]

More specifically, the test substance may be identified as a substance for preventing, alleviating or treating lung injury if the functional capillary ratio has increased by 1.1 times or more, 1.2 times or more, 1.3 times or more, 1.4 times or more, 1.5 times or more, 1.6 times or more, 1.7 times or more, 1.8 times or more, 1.9 times or more, 2 times or more, 2.1 times or more, 2.2 times or more, 2.3 times or more, 2.4 times or more, 2.5 times or more, 2.6 times or more, 2.7 times or more, 2.8 times or more, 2.9 times or more or 3 times or more in a lung injury model treated with the test substance as compared to a lung injury model not treated with the test substance.

In another aspect, the present disclosure provides a method for screening a substance alleviating microcirculatory disorder in the lung, which includes: (a) a step of preparing a lung injury model; (b) a step of treating the lung injury model with a test substance; (c) a step of measuring the change in the expression or activity of macrophage-1 antigen (Mac-1) in neutrophils within pulmonary capillaries of the lung injury model caused by the test substance; and (d) a step of identifying whether the test substance increases a functional capillary ratio, which is the ratio of the area of functional capillaries through which erythrocytes pass to the total capillary area of the lung injury model. The lung injury model, the microcirculation, the microcirculatory disorder, the neutrophils, the macrophage-1 antigen, the relative expression level and the functional capillary ratio are the same as described above.

The step (c) may include a step of comparing the expression or activity of macrophage-1 antigen in neutrophils within pulmonary capillaries before and after treating the lung injury model with the test substance. Alternatively, the step (c) may include a step of comparing the expression or activity of macrophage-1 antigen in neutrophils within pulmonary capillaries of a lung injury model treated with the test substance with that of a lung injury model not treated with the test substance.

In addition, the screening method may further include a step of, if the expression or activity of macrophage-1 antigen is decreased as compared to before the treatment with the test substance as a result of measuring the expression or activity in the step (c) or if a functional capillary ratio, which is the ratio of the area of functional capillaries through which erythrocytes pass to the total capillary area of the lung injury model, is increased, identifying the test substance as a substance for preventing, alleviating or treating lung injury.

In the step of identifying as a substance for preventing, alleviating or treating lung injury, the test substance may be identified as a substance for preventing, alleviating or treating lung injury if the expression or activity of macrophage-1 antigen has decreased by about 10% or more as a result of measuring the expression or activity in the step (c). That is to say, if the expression or activity has decreased by about 10% or more when the expression or activity of macrophage-1 antigen in neutrophils within pulmonary capillaries of the lung injury model treated with the test substance was compared with the expression or activity of macrophage-1 antigen in neutrophils within pulmonary capillaries of the lung injury model before treatment with the test substance, it may be identified as a substance for preventing, alleviating or treating lung injury. Alternatively, if the expression or activity has decreased by about 10% or more when the expression or activity of macrophage-1 antigen in neutrophils within pulmonary capillaries of the lung injury model treated with the test substance was compared with the expression or activity of macrophage-1 antigen in neutrophils within pulmonary capillaries of the lung injury model not treated with the test substance, it may be identified as a substance for preventing, alleviating or treating lung injury. For example, when the expression or activity of macrophage-1 antigen has decreased by 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more or 90% or more as compared to before the treatment with the test substance, it may be identified as a substance for preventing, alleviating or treating lung injury, although not being limited thereto. The degree of the expression or activity is measured within statistical significance. The statistical significance refers to significant difference achieved through biological statistical analysis, quantitatively with a p-value smaller than 0.05.

In an exemplary embodiment, the degree of the expression or activity of macrophage-1 antigen may be identified by known techniques, e.g., reverse transcription polymerase chain reaction (RT-PCR), ELISA, western blot or immune blot, although not being limited thereto.

In the step of identifying as a substance for preventing, alleviating or treating lung injury, the test substance may be identified as a substance for preventing, alleviating or treating lung injury if the functional capillary ratio, i.e. the ratio of the area passed by erythrocytes to the total capillary area, has increased by about 1.1 times or more in the step (c). Specifically, the test substance may be identified as a substance for preventing, alleviating or treating lung injury if the functional capillary ratio (FCR) according to Formula 1 of a lung injury model has increased by 1.1 times or more after treatment with the test substance as compared to before the treatment with the test substance. Alternatively, the test substance may be identified as a substance for preventing, alleviating or treating lung injury if the functional capillary ratio has increased by about 1.1 times or more in a lung injury model treated with the test substance as compared to a lung injury model not treated with the test substance.

Functional capillary ratio=functional capillary area/total capillary area [Formula 1]

More specifically, the test substance may be identified as a substance for preventing, alleviating or treating lung injury if the functional capillary ratio has increased by 1.1 times or more, 1.2 times or more, 1.3 times or more, 1.4 times or more, 1.5 times or more, 1.6 times or more, 1.7 times or more, 1.8 times or more, 1.9 times or more, 2 times or more, 2.1 times or more, 2.2 times or more, 2.3 times or more, 2.4 times or more, 2.5 times or more, 2.6 times or more, 2.7 times or more, 2.8 times or more, 2.9 times or more or 3 times or more in a lung injury model treated with the test substance as compared to a lung injury model not treated with the test substance.

In an exemplary embodiment, the present disclosure may provide the macrophage-1 antigen in neutrophils within pulmonary capillaries as a biomarker for diagnosis of pulmonary microcirculatory disorder. In another exemplary embodiment, the present disclosure may provide a composition or a kit for diagnosis of pulmonary microcirculatory disorder. In another exemplary embodiment, the present disclosure may provide a method for providing information for diagnosis of pulmonary microcirculatory disorder.

Specifically, the present disclosure may provide a method for providing information for diagnosis of pulmonary microcirculatory disorder, which includes: a step of measuring the expression or activity of macrophage-1 antigen (Mac-1) in neutrophils isolated from the pulmonary capillaries of a test subject; and a step of identifying a functional capillary ratio, which is the ratio of the area of functional capillaries through which erythrocytes pass to the total capillary area of the lung of the test subject.

In an exemplary embodiment, the method may further include a step of comparing the degree of expression or activity of macrophage-1 antigen in the neutrophils isolated from the pulmonary capillaries of the test subject with the degree of expression or activity of macrophage-1 antigen in the neutrophils isolated from the pulmonary capillaries of a normal control group.

Also, in an exemplary embodiment, the method may further include a step of providing information that the subject has pulmonary microcirculatory disorder when the degree of expression or activity of macrophage-1 antigen in the neutrophils isolated from the pulmonary capillaries of the test subject is higher than the degree of expression or activity of macrophage-1 antigen in the neutrophils isolated from a normal control group. For example, it may be identified as pulmonary microcirculatory disorder when the expression or activity of macrophage-1 antigen in neutrophils isolated from the pulmonary capillaries of a test subject is higher than the expression or activity of macrophage-1 antigen in neutrophils isolated from the pulmonary capillaries of a normal control group by 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more or 90% or more. The degree of expression or activity is measured within statistical significance. The statistical significance refers to significant difference achieved through biological statistical analysis, quantitatively with a p-value smaller than 0.05.

In an exemplary embodiment, the method may further include a step of comparing the functional capillary ratio, which is the ratio of the area of functional capillaries through which erythrocytes pass to the total capillary area of the lung of the test subject, with the functional capillary ratio of a normal control group.

In another exemplary embodiment, the method may include a step of providing information that the test subject has pulmonary microcirculatory disorder when the functional capillary ratio, which is the ratio of the area of functional capillaries through which erythrocytes pass to the total capillary area of the lung of the test subject, is lower than that of a normal control group. The functional capillary ratio, which is the ratio of the area of functional capillaries through which erythrocytes pass to the total pulmonary capillary area, may be a functional capillary ratio (FCR) according to Formula 1. For example, when the functional capillary ratio, which is the ratio of the area of functional capillaries through which erythrocytes pass to the total capillary area of the lung of the test subject, is 70% or lower, 65% or lower, 60% or lower, 55% or lower, 50% or lower, 45% or lower, 40% or lower, 35% or lower, 30% or lower, 25% or lower, 20% or lower, 15% or lower, 10% or lower or 5% or lower, it may be determined as pulmonary microcirculatory disorder. However, the ratio for determining microcirculatory disorder may vary depending on the organ of the subject in which the capillaries are distributed, and is not limited to the above ranges. The functional capillary ratio, which is the ratio of the area of functional capillaries through which erythrocytes pass to the total pulmonary capillary area, is measured within statistical significance. The statistical significance refers to significant difference achieved through biological statistical analysis, quantitatively with a p-value smaller than 0.05.

In another aspect, the present disclosure may provide a composition for diagnosis of pulmonary microcirculatory disorder, which contains a reagent for detecting mRNAs or proteins of macrophage-1 antigen (Mac-1) in neutrophils within pulmonary capillaries.

In another aspect, the present disclosure may provide a kit for diagnosis of pulmonary microcirculatory disorder, which includes a reagent for detecting mRNAs or proteins of macrophage-1 antigen (Mac-1) in neutrophils within pulmonary capillaries. The kit may further include an instruction describing a method for providing the information for diagnosis of microcirculatory disorder.

In an exemplary embodiment, the reagent for detecting mRNAs or proteins of macrophage-1 antigen included in the composition or kit for diagnosis of pulmonary microcirculatory disorder may include one or more of a primer and a probe binding specifically to macrophage-1 antigen. In an exemplary embodiment, the reagent for detecting proteins of macrophage-1 antigen may include one or more of an antibody and a probe binding specifically to macrophage-1 antigen.

In the present specification, the “probe” includes a polynucleotide having a base sequence capable of binding complementarily to the target site of a gene, a variant thereof, or the polynucleotide with a label attached thereto.

In the present specification, the “primer” refers to a polynucleotide having a base sequence capable of binding complementarily to the terminal of a specific region of a gene to amplify the target site of the gene through PCR or a variant thereof. The primer does not have to be completely complementary to the specific area region, and is sufficient as long as it can form a double-stranded structure by hybridizing to the terminal.

In the present specification, the “hybridization” refers to the formation of a duplex structure through pairing of two single-stranded nucleic acids having complementary base sequences.

The hybridization may occur not only when the sequences of the single-stranded nucleic acids are completely complementary to each other (perfect match) but also when there are some mismatches.

In the present specification, the “polynucleotide” refers to a polymer of a plurality of nucleotides. The term is used in a broad concept, including oligonucleotides which are polymers of tens of nucleotides.

Hereinafter, the present disclosure is described more specifically through examples and experimental examples. However, the following example and experimental examples are provided only for the purpose of illustrating the present disclosure and the scope of the present disclosure is not limited by them.

In the following example and experimental example, all animal experiments were performed in accordance with the standard guidelines for the care and use of laboratory animals and were approved by the Institutional Animal Care and Use Committee (IACUC) of KAIST (Protocol Nos. KA2014-30 and KA2016-55).

Also, in the following example and experimental example, all data are presented as mean±SD or median±interquartile range, as appropriate, to represent the values of each group. The statistical difference between means or medians was determined by unpaired 2-tailed Student's t-test, Mann-Whitney test, one-way ANOVA with post-hoc Holm-Sidak's multiple comparisons, or Kruskal-Wallis test with post-hoc Dunn's multiple comparison tests, as appropriate. Statistical significance was set at P<0.05 and analysis was performed with Prism 6.0 (Graph Pad).

### [Example 1] Preparation of Sepsis-Induced Acute Lung Injury Mouse Model

### [Example 1-1] Preparation of Mouse Model for Quantitation of Microcirculation

For quantification of microcirculation, a sepsis-induced acute lung injury mouse model was prepared as follows.

All mice used in the examples were individually housed in ventilated and temperature (22.5° C.- and humidity (52.5%)-controlled cages under 12:12h light:dark cycle and were provided with standard diet and water ad libitum. 8- to 20-weeks-old male mouse (20-30 g) were used for the experiment. C57BL/6N mice were purchased from OrientBio (Suwon, Korea) and Tie2-GFP mice (Stock No. 003658, Jackson Laboratory) where GFP is expressed under an endothelium-specific Tie2 promoter were purchased from Jackson Laboratory.

To generate a sepsis-induced acute lung injury (ALI) mouse model, high-dose LPS was administered to the Tie2-GFP mice (hereinafter, Tie2-GFP-ALI mouse model).

For the high-dose LPS model, LPS (10 mg/kg, E. coli serotype 055:B5, L2880, Sigma-Aldrich) was intraperitoneally administered to the peritoneum of the Tie2-GFP mice 3-6 hours before capillary imaging. As a control group, the same amount of PBS was injected into the peritoneum of the mice.

### [Example 1-2] Preparation of Mouse Model for Diagnosis of Microcirculatory Disorder

For diagnosis of microcirculatory disorder, a sepsis-induced acute lung injury mouse model was prepared in the same manner as in Example 1-1.

But, all the mice used in this example were LysM^{GFP/+} mice, rather than Tie2-GFP mice, provided by Professor Minsu Kim at University of Rochester (hereinafter, referred to as LysM^{GFP/+} mouse model).

To generate a sepsis-induced acute lung injury (ALI) mouse model, high-dose LPS was administered to the LysM^{GFP/+} mice.

For the high-dose LPS model, LPS (10 mg/kg, E. coli serotype 055:B5, L2880, Sigma-Aldrich) was intraperitoneally administered to the peritoneum of the LysM^{GFP/+} mice 3-6 hours before capillary imaging (hereinafter, referred to as ALI mouse models; LPS 3h mouse model: 3 hours after administration of LPS, LPS 6h mouse model: 6 hours after administration of LPS). As a control group, the same amount of PBS was injected into the peritoneum of the mice (hereinafter, referred to as control group or PBS mouse model).

### [Example 1-3] Preparation of Mouse Model for Studying Composition for Preventing or Treating Lung Injury Disease

For studying a composition for preventing or treating lung injury disease, a sepsis-induced acute lung injury mouse model was prepared in the same manner as in Example 1-1.

But, all the mice used in this example were LysM^{GFP/+} mice, rather than Tie2-GFP mice, provided by Professor Minsu Kim at University of Rochester (hereinafter, referred to as LysM^{GFP/+} mouse model).

As a sepsis-induced acute lung injury (ALI) mouse model, a high-dose LPS mouse model administered to the LysM^{GFP/+} mouse or a CLP (cecal ligation and puncture) mouse model was utilized.

For the high-dose LPS model, LPS (10 mg/kg, E. coli serotype 055:B5, L2880, Sigma-Aldrich) was intraperitoneally administered to the peritoneum of the LysM^{GFP/+} mice 3-6 hours before capillary imaging (hereinafter, referred to as ALI mouse models; LPS 3h mouse model: 3 hours after administration of LPS, LPS 6h mouse model: 6 hours after administration of LPS). As a control group, the same amount of PBS was injected into the peritoneum of the mice (hereinafter, referred to as control group or PBS mouse model).

CLP model was performed by a single experienced operator according to a previously described method. Specifically, 75% of the cecum of the LysM^{GFP/+} mouse was ligated with 6-0 black silk and single puncture with a dual hole in the distal cecum was made with a 21-gauge needle. After the puncture, the cecum was gently squeezed to confirm the patency of the puncture hole for extrusion of feces. The cecum was replaced to the abdominal cavity and the abdominal incision was closed with 4-0 black silk. A normal group (sham group) underwent the same surgical procedure except the cecal ligation and puncture.

### [Example 1-4] Preparation of Neutrophil-Depleted Models (N-Dep Model and N-Dep+LPS Model)

Neutrophil-depleted mouse models (hereinafter, neutrophil-depleted models) were prepared to investigate the effect of neutrophils on the lung injury caused by pulmonary microcirculatory disorder. Specifically, a neutrophil-depleted mouse model with no lung injury was prepared by intraperitoneally injecting 200 μg of anti-Ly6G+ monoclonal antibody (Clone 1A8, 551459, BD Biosciences) to the LysM^{GFP/+} mice of Example 1-3 24 hours before intravital lung imaging (hereinafter, N-Dep model). In addition, a neutrophil-depleted lung injury mouse model was prepared by intraperitoneally injecting 200 μg of anti-Ly6G+ monoclonal antibody (Clone 1A8, 551459, BD Biosciences) to the acute lung injury mouse model of Example 1-3 24 hours before the preparation of the acute lung injury mouse model in Example 1-3 (hereinafter, N-Dep+LPS model).

### [Example 2] Staining of Erythrocytes and Vasculature, Labeling of Neutrophils and Intravital Lung Imaging

(1) Staining of Erythrocytes and Vasculature

For imaging of microcirculation in vivo, the erythrocytes and vasculature of the mouse model of Example 1-1 were fluorescence-stained. Specifically, erythrocytes were acquired by cardiac puncture and then labeled according to the method described in the product information sheet. At this time, erythrocytes were fluorescence-labeled Vybrant DiD (V22887, ThermoFisher Scientific). Then, adoptive transfer was performed by injecting 50 million counts of DiD-labeled erythrocytes via a vascular catheter into the tail vein of the Tie2-GFP-ALI mouse model of Example 1-1 right before imaging.

In addition, to visualize the blood vessel with a fluorescent dye, FITC (molecular weight 2 MDa, Sigma-Aldrich)- or tetramethylrhodamine (TMR)-conjugated dextran dye was injected to the Tie2-GFP-ALI mouse model of Example 1-1 via the same vascular catheter.

The procedure of injecting the DiD-labeled erythrocytes or the FITC- or TMR-conjugated dextran dye to the mouse model is described below intravital lung imaging.

(2) Labeling of Neutrophils of Mouse Model of Example 1-2

For imaging of the motion of neutrophils in vivo, anti-Ly6G+ monoclonal antibody (Clone 1A8, 551459, BD Biosciences) conjugated with the fluorophore Alexa Fluor 555 or 647 (A-20005/A-20006, ThermoFisher Scientific) was injected via the tail vein of the mouse model of Example 1-2 2 hours before imaging for labeling of neutrophils.

In addition, the erythrocytes and vasculature of the mouse model of Example 1-2 were fluorescence-stained. Specifically, erythrocytes were acquired by cardiac puncture and then fluorescence-labeled Vybrant DiD (V22887, ThermoFisher Scientific) according to the method described in the product information sheet. Then, 50 million counts of DiD-labeled erythrocytes were injected via a vascular catheter into the tail vein of the mouse model of Example 1-2 right before imaging. In addition, to visualize the vasculature, FITC (molecular weight 2 MDa, Sigma-Aldrich)- or tetramethylrhodamine (TMR)-conjugated dextran dye was injected to the Tie2-GFP-ALI mouse model of Example 1-2 via the same vascular catheter.

The procedure of injecting the fluorophore-labeled anti-Ly6G+ monoclonal antibody, DiD-labeled erythrocytes or the FITC- or TMR-conjugated dextran dye to the mouse model is described below intravital lung imaging.

(3) Labeling of Neutrophils of Mouse Model of Example 1-3

To visualize the molecular expression of pulmonary sequestered neutrophils in vivo, 25 μg of CD11b (Clone M_{1/70}, 553307, BD Biosciences) and 25 μg of CD18 (Clone GAME-46, 555280, BD Biosciences) conjugated with the fluorophore Alexa Fluor 555 (A-20005, ThermoFisher Scientific) was injected via the tail vein of the mouse model of Example 1-3 2 hours before imaging for labeling of neutrophils.

In addition, the erythrocytes and vasculature of the mouse model of Example 1-3 were fluorescence-stained. Specifically, erythrocytes were acquired by cardiac puncture and then fluorescence-labeled Vybrant DiD (V22887, ThermoFisher Scientific) according to the method described in the product information sheet. Then, 50 million counts of DiD-labeled erythrocytes were injected via a vascular catheter into the tail vein of the mouse model of Example 1-3 right before imaging. In addition, to visualize the vasculature, FITC (molecular weight 2 MDa, Sigma-Aldrich)- or tetramethylrhodamine (TMR)-conjugated dextran dye was injected to the Tie2-GFP-ALI mouse model of Example 1-3 via the same vascular catheter.

The procedure of injecting the fluorophore-labeled CD11b and CD18, DiD-labeled erythrocytes or the FITC- or TMR-conjugated dextran dye to the mouse model is described below intravital lung imaging.

(4) Intravital Lung Imaging

Intravital lung imaging was performed as follows.

Specifically, after anesthetizing the Tie2-GFP-ALI mouse model, control mouse model and normal (sham) mouse model of Example 1-1, the LysM^{GFP/+} mouse model, ALI mouse model, LPS 3h mouse model, LPS 6h mouse model and PBS mouse model of Example 1-2, the LysM^{GFP/+} mouse model, ALI mouse model, PBS mouse model and normal (sham) mouse model of Example 1-3, the N-Dep mouse model and N-Dep+LPS mouse model of Example 1-4, and a Mac-1-inhibited mouse model of Experimental Example 12-1 with ketamine (80 mg/kg) and xylazine (12 mg/kg), intubation was performed using a 20-gauge vascular catheter as a lightning guidewire and was connected to a mechanical ventilator (MouseVent, Kent Scientific). Ventilation was conducted in the setting of an inspiratory pressure 24-30 mmHg, a respiratory rate of 120-130 breaths per minute, and a positive-end expiratory pressure (PEEP) of 2 cmH₂O. 2% isoflurane was delivered to maintain anesthesia status, and pulse oximetry was applied to monitor oxygenation and survival status. A thermal probe of a homeothermic system (RightTemp, Kent Scientific) was introduced into the rectum, and a feedback-regulated heating pad was used to maintain body temperature at 37.0° C. The tail vein was cannulated with a 30-gauge needle attached to a PE-10 tube for intravenous injection of the dye, erythrocytes and neutrophils of (1). Then, the mice were positioned in right lateral decubitus, which was followed by left thoracotomy. The skin and muscle were dissected until rib exposure, and incision was made between the 3rd and 4th ribs to expose the pleura. After the thoracotomy, an imaging window described below in the experimental examples was applied to the surface of the pleura, and negative suction pressure was applied by a pump (DOA-P704-AA, GAST) and a regulator (NVC 2300a, EYELA) via a tube connected to the lung imaging window.

### [Experimental Example 1] Imaging of Pulmonary Microcirculation

### [Experimental Example 1-1] Imaging of Pulmonary Microcirculation by Staining of Erythrocytes

To visualize pulmonary microcirculation in vivo through the pulmonary imaging window, a custom-built video-rate laser-scanning confocal microscopy system was implemented.

Imaging System

Specifically, three continuous laser modules (488 nm (MLD488, Cobolt), 561 nm (Jive, Cobolt) and 640 nm (MLD640, Cobolt)) were utilized as excitation light sources for multi-color fluorescence imaging. Laser beams were collinearly integrated by diachronic beam splitters (DBS1; FF593-Di03, DSB2; FF520-Di02, Semrock) and transferred to a laser beam scanner by multi-edge diachronic beam splitters (DBS3; Di01-R405/488/561/635, Semrock). The laser scanning section consisted of 2 axes: X-axis scanning with a rotating polygonal mirror with 36 facets (MC-5, aluminum-coated, Lincoln Laser) and Y-axis scanning with galvanometer scanning mirror (6230H, Cambridge Technology). The two-dimensional raster scanning laser beam was transferred to the lung of the Tie2-GFP-ALI mouse model of Example 1-1 through commercial objective lenses (LUCPLFLN, 20×, NA 0.45, Olympus, LUCPLFLN, 40×, NA 0.6, Olympus, LCPLFLN100×LCD, 100×, NA 0.85, Olympus). The fluorescence signals emitted from the lung of the mouse model on a XYZ translational 3D stage (3DMS, Sutter Instrument) were epi-detected by the objective lenses. De-scanned three-color fluorescence signals were spectrally divided by diachronic beam splitters (DBS4; FF560-Di01, DBS5; FF649-Di01, Semrock) and then detected by a photomultiplier (PMT; R9110, Hamamatsu) through bandpass filters (BPF1; FF02-525/50, BPF2; FF01-600/37, BPF3; FF01-685/40, Semrock). The voltage output of each PMT was digitalized by a 3-channel frame grabber (Solios, Matrox) with 8-bit resolution at a sampling rate of 10 MHz. Video-rate movies were displayed and recorded in real time using a customized imaging software based on Matrox Imaging Library (MIL9, Matrox) and Visual C# at a frame rate of 30 Hz and a frame size of 512×512 pixels.

Image Processing

The images imaged using the imaging system were displayed and stored at an acquisition rate of 30 frames per second with 512×512 pixels per frame. The real-time image frames were averaged over 30 frames using a MATLAB (Mathworks) code to improve contrast and signal-to-noise ratio. To minimize motion artifact, each frame was processed with an image registration algorithm prior to the averaging. Image rendering with three-dimensional reconstruction, track analysis of erythrocytes and neutrophils and plotting of track displacement were conducted using IMARIS 8.2 (Bitplane).

A result of imaging the pulmonary microcirculation of the control mouse model of Example 1-1 using the imaging system described above and processing the obtained mages as described above is shown in FIG. 2.

As shown in FIG. 2, by using the method for quantitation of microcirculation and the apparatus for measuring microcirculation according to an aspect of the present disclosure, rapidly flowing erythrocytes (DiD-labeled erythrocytes) were clearly visible inside the pulmonary capillaries in which the erythrocytes were labeled with GFP in real time, enabling the acquisition of a plurality of motion images of erythrocytes flowing through the capillaries and spatiotemporal information on the flowing trajectory and velocity of individual erythrocytes.

### [Experimental Example 1-2] Imaging of Pulmonary Microcirculation by Labeling of Neutrophils

Pulmonary microcirculation was imaged in the same manner as in Experimental Example 1-1, except that the LysM^{GFP/+} mouse model of Example 1-2 was used instead of the Tie2-GFP-ALI mouse model of Example 1-1.

A result of imaging the pulmonary microcirculation of the LysM^{GFP/+} mouse model of Example 1-2 to which LPS was not administered using the imaging system described above and processing the obtained mages as described above is shown in FIG. 7.

As shown in FIG. 7, it was confirmed that circulation in the pulmonary capillaries resumes after neutrophils in the upper region (\hat{ }, blue) and the lower region (*, red) were squeezed through the pulmonary capillaries, and that neutrophils were excessively entrapped in the pulmonary capillaries of the LysM^{GFP/+} mouse model. In addition, it was confirmed that, whereas the circulating cells, which were assumed to be erythrocytes, could not flow through the capillaries during the period in which the capillaries were obstructed, the blood flow resumed after the neutrophils passed through the capillaries.

In contrast, in the sepsis-induced acute lung injury mouse model, the functional capillary ratio (FCR; calculated as a ratio of the functional capillary area to the total capillary area) was decreased during the early stage of acute lung injury. The capillary obstruction found in the lung injury mouse model was induced by the objects inside the capillaries that could represent the primary pathophysiological mechanism underlying the decreased FCR. From the result shown in FIG. 7, it can be seen that the objects that induced the obstruction could be neutrophils because neutrophils respond rapidly to systemic inflammation.

Therefore, it can be seen that neutrophil in capillaries, specifically the entrapment of neutrophils in capillaries, are associated with microcirculatory disorder, particularly sepsis. Accordingly, the method for providing information and the apparatus for diagnosis of microcirculatory disorder according to an aspect of the present disclosure can provide a plurality of motion images of neutrophils passing through capillaries by clearly visualizing the motion of neutrophils inside the capillaries and allow easy and convenient diagnosis of microcirculatory disorder in a subject by acquiring information about the motion of each neutrophil.

### [Experimental Example 2] Quantitation of Microcirculation Based on Functional Capillary Ratio (FCR)

For quantification of microcirculation in a subject based on a functional capillary ratio (FCR), functional capillary imaging analysis was performed using a real-time movie of DiD-labeled erythrocytes flowing in capillaries, which was acquired using the imaging system and image processing of Experimental Example 1-1. After splitting the colors of the movie, sequential images of channels detecting DiD was processed by a median filter with a radius of 2 pixels to enhance the signal-to-noise ratio. The maximal intensity projection of 600-900 frames (20-30 seconds) was generated to show the functional capillaries perfused by erythrocytes. The functional capillary ratio (FCR) was calculated according to Formula 1.

Functional capillary ratio=functional capillary area/total capillary area [Formula 1]

In Formula 1, the total capillary area means the vessel area detected by Tie2 or dextran signaling, and the functional capillary area means the area traveled by DiD-labeled erythrocytes. All image processing to calculate the functional capillary ratio was performed with ImageJ (https://imagej.nih.gov/ij/), and the result is shown in FIG. 3 and FIG. 4.

FIG. 4 is a graph showing the functional capillary ratio calculated by summing the spaces through which erythrocytes pass by time domain.

As shown in FIG. 3 and FIG. 4, microcirculation can be quantified by calculating the functional capillary ratio using the method and apparatus for quantitation of microcirculation according to an aspect of the present disclosure.

### [Experimental Example 3] Comparison of Functional Capillary Ratio of Lung Injury Mouse Model and Control Group

The functional capillary ratio was compared for the Tie2-GFP-ALI mouse model in which sepsis was induced by LPS administration in Example 1-1 and a control group to which PBS was administered instead of LPS. Pulmonary microcirculation was imaged also for the control mouse model in the same manner as in Experimental Example 1-1 and Experimental Example 2 and the obtained images were analyzed. As a result, although no significant difference was found in the mean velocity of erythrocytes between the control mouse model and the lung injury mouse model, the erythrocyte perfusion pattern changed dramatically in the lung injury mouse model. In addition, the erythrocytes in sequential images from 600 frames (20 seconds) were presented in a maximal intensity projection to quantify the perfusion area of the erythrocytes.

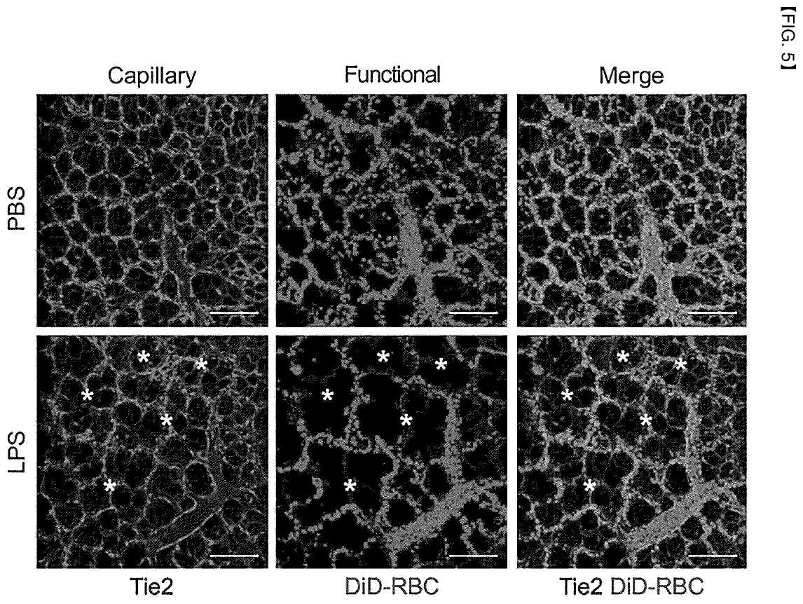

The functional capillary ratio (FCR) of the control mouse model and the lung injury mouse model (Tie2-GFP-ALI mouse model) was calculated according to Formula 1 as in Experimental Example 2, and the result is shown in FIG. 5 and FIG. 6 (n (number of fields)=30, 10 FOV (field of view) per mouse, 3 mice per each group, P=0.8157, *P<0.05, two-tailed t-test).

As shown in FIG. 5, whereas the control group model exhibited widespread and diffuse characteristics of perfusion, the perfusion in the lung injury mouse model (Tie2-GFP-ALI mouse model) was more concentrated and overlapped with arterioles and a few capillaries. Unlike the control group model, the acute lung injury mouse model (Tie2-GFP-ALI mouse model) showed dead space (white asterisks in FIG. 5) where the erythrocyte could not pass through.

Also, as shown in FIG. 6A and FIG. 6B, whereas there was no difference in total capillary area between the control group model and the lung injury mouse model (Tie2-GFP-ALI mouse model) (FIG. 6A), the functional capillary ratio (FCR) was decreased by 50% or more in the acute lung injury mouse model (Tie2-GFP-ALI mouse model) as compared to the control group model because the functional capillary area through which the erythrocytes pass was decreased rapidly (FIG. 6B). This represents abnormal perfusion in the sepsis-induced acute lung injury mouse model (Tie2-GFP-ALI mouse model).

Furthermore, arterial blood gas analysis was performed to assess the oxygen partial pressure and carbon dioxide partial pressure in the arterial blood of the control group model and the lung injury mouse model (Tie2-GFP-ALI mouse model). Specifically, a 1-mL syringe with a 22-gauge needle was coated with heparin and introduced into the left ventricle of the heart of the control group model (PBS, n=6) and the lung injury mouse model (LPS, n=16). Then, about 200 μL of blood was sampled and analyzed with an i-STAT handheld blood analyzer (G3 cartridge, Abbott Point of Care Inc.). The mice were euthanized in a CO₂ chamber right after the blood sampling. The arterial blood gas analysis result is shown in FIG. 6C and FIG. 6D (*P<0.05, Mann-Whitney test). As shown in FIG. 6C and FIG. 6D, the lung injury mouse model (Tie2-GFP-ALI mouse model) showed decreased oxygen partial pressure (FIG. 6C) and increased carbon dioxide partial pressure (FIG. 6D) in the arterial blood as compared to the control group model. It was confirmed that the decrease of functional capillary ratio in the lung injury mouse model (Tie2-GFP-ALI mouse model) was due to hypoxemia and hypercapnia.

Accordingly, by using the method for quantitation of microcirculation and the apparatus for measuring microcirculation according to an aspect of the present disclosure, the microcirculation in a subject can be quantified easily and conveniently in vivo based on the functional capillary ratio, and microcirculatory disorder can be identified accurately and quickly based on the quantification result.

### [Experimental Example 4] Comparison of Motion of Neutrophils for Lung Injury Mouse Model and Control Group Model

The relationship between the motion of neutrophils in capillaries and microcirculatory disorder was confirmed in Experimental Example 1-2. In order to visualize the motion of neutrophils in the ALI mouse model (LPS) and the control group model (PBS) prepared in Example 1-2, a customized video-rate laser scanning confocal microscopy system was implemented in the same manner as in Experimental Examples 1-1 and 1-2. The result of intravital imaging is shown in FIG. 8.

As shown in FIG. 8, whereas neutrophils passed through the pulmonary capillaries in the control mouse model, the flow of the cells in the pulmonary microcirculation was interrupted in numerous spots in the ALI mouse model.

In addition, based on the wide field image processing result of FIG. 8, the number of neutrophils per unit area (512×512 μm) was compared for the ALI mouse model (LPS) and the control mouse model (PBS). The result is shown in FIG. 9. As shown in FIG. 9, whereas the number of neutrophils was about 10 cells/field for the control group, the number of neutrophils for the ALI mouse model was about 20 times larger for the ALI mouse model as compared to the control group model, as about 200 cells/field (n (number of analyzed fields)=30, 10 FOV (field of view) per mouse, 3 mice per each group, *P<0.05, two-tailed t-test, data are means±s.d.). This means that the number of neutrophils imaged by pulmonary microcirculation imaging in Experimental Example 1-2 is large because the neutrophils are entrapped in the capillaries without circulating due to acute lung injury and, thus, are more likely to be found in the images imaged with time intervals. Through this, it was confirmed that, when innate immune cells are recruited during early inflammation, the neutrophils are the primary obstacle in the microcirculation in pulmonary capillaries.

Therefore, it can be seen that the sequestration (entrapment) of neutrophils in capillaries is associated with microcirculatory disorder. Accordingly, the method for providing information and the apparatus for diagnosis of microcirculatory disorder according to an aspect of the present disclosure can provide a plurality of motion images of neutrophils passing through capillaries by clearly visualizing the motion of neutrophils inside the capillaries and allow easy and convenient diagnosis of microcirculatory disorder in a subject by acquiring information about the motion of neutrophils.

### [Experimental Example 5] Comparative Analysis of Motion (Track) of Neutrophils in Lung Injury Mouse Model and Control Group Model

It was confirmed in Experimental Example 4 that the entrapment of neutrophils in capillaries can lead to microcirculatory disorder such as lung injury. In this example, the motion (track) of neutrophils was comparatively analyzed for the acute lung injury mouse models (LPS 3h mouse model and LPS 6h mouse model) in which sepsis was induced by LPS administration in Example 1-2 and the control mouse model to which PBS was administered instead of LPS.

Tracking of Neutrophils Through Time-Lapse Imaging

Specifically, a customized video-rate laser scanning confocal microscopy system was implemented in the same manner as in Experimental Examples 1-1 and 1-2 and the pulmonary microcirculation of mouse models was intravitally imaged at a slow rate for 30 minutes. The result is shown in FIGS. 10A and 10B.

As shown in the track images of the neutrophils time-lapse imaged for 30 minutes (FIG. 10A), whereas the number of neutrophils remaining at a specific location for 30 minutes was very small for the control group (PBS), the number of neutrophils remaining at a specific location for 30 minutes was increased for the LPS 3h mouse model as compared to the control group, and the neutrophils remained at a specific location for 30 minutes over the total capillary area for the LPS 6h mouse model.

This is also confirmed from the track displacement of the neutrophils shown in FIG. 10B. The track displacement of neutrophils was increased rapidly in the LPS 3h mouse model as compared to the control group, suggesting that the motility of neutrophils in capillaries was increased 3 hours after the LPS administration. However, the track displacement of neutrophils was decreased in the LPS 6h mouse model as the motility of neutrophils was decreased due to aggravation of inflammation owing to microcirculatory disorder.

That is to say, from FIGS. 10A and 10B, given that the flow velocity of the erythrocytes was high (>500 μm/s), it can be seen that the neutrophils detected continuously for 2 minutes or longer were not flowing but were sequestered at a specific area of the capillary due to lung injury (i.e., crawling or marginating inside the blood vessel).

Comparison of Degree of Neutrophil Sequestration

The degree of neutrophil sequestration was compared for the control group and the lung injury mouse model from the time-lapse imaging result (FIGS. 10A and 10B), and the result is shown in FIG. 11. In FIG. 11, the x-axis represents sequestration time and the y-axis represents the number of tracks shown in FIG. 10A and FIG. 10B depending on time.

As shown in FIG. 11, whereas the number of tracks was about 100 within 1 minute for the control group, the number of tracks was about 300 or more between 29 and 30 minutes for the LPS 6h mouse model, meaning that more neutrophils are entrapped or sequestered in the microcirculation as compared to the control group. That is to say, it was confirmed that, whereas most neutrophils are sequestered in very short time for the control group, the proportion of sequestered neutrophils was remarkably increased for the lung injury mouse models (LPS 3h and LPS 6h) as compared to the control group.

### [Experimental Example 6] Comparative Analysis of Dynamic Behavior of Neutrophils in Lung Injury Mouse Model and Control Group Model

It was confirmed in Experimental Example 5 that the motion of neutrophils in capillaries was decreased in the lung injury mouse model as compared to the control group model due to sequestration. Based on the data acquired in Experimental Examples 1, 4 and 5, the dynamic behavior of neutrophils (sequestration time, track displacement length, track length, track velocity and track meandering index) was comparatively analyzed for the acute lung injury mouse models in which sepsis was induced by LPS administration in Example 1-2 (LPS 3h mouse model and LPS 6h mouse model) and the control mouse model to which PBS was administered instead of LPS, and the result is shown in FIGS. 12A-12E (n (number of tracks)=466 (PBS), 794 (LPS 3h) and 1076 (LPS 6h), 3 mice per each group, *P<0.05, Kruskal-Wallis test with post-hoc Dunn's multiple comparison, data are medians±interquartile range).

First, the sequestration time (FIG. 12A) was about 3 minutes for the control group (PBS), about 8 minutes for the LPS 3h mouse model and about 18 minutes for the LPS 6h mouse model. The neutrophil sequestration time was longer for the lung injury mouse models (LPS administration groups) as compared to the control group (PBS). The sequestration time was about 2 times longer for the LPS 6h mouse model as compared to the LPS 3h mouse model.

The track displacement length (FIG. 12B) was about 3 μm for the control group (PBS), about 8 μm for the LPS 3h mouse model and about 4 μm for the LPS 6h mouse model. The track displacement length was increased by about 2-3 times for the LPS 3h mouse model as compared to the control group (PBS). At 6 hours after the LPS administration, the track displacement length of the LPS 6h mouse model was decreased again to a level comparable to that of the control group.

The track length (FIG. 12C) was about 10 μm for the control group (PBS), about 23 μm for the LPS 3h mouse model and about 15 μm for the LPS 6h mouse model. Similarly to the track displacement length, the track length was increased by about 2 times or more for the LPS 3h mouse model as compared to the control group (PBS), and then decreased again for the LPS 6h mouse model to a level comparable to that of the control group.

The track velocity (FIG. 12D) was about 1.0 μm/m for the control group (PBS), about 1.9 μm/m for the LPS 3h mouse model and about 0.8 μm/m for the LPS 6h mouse model. The track velocity was increased by about 1.5 times or more for the LPS 3h mouse model as compared to the control group (PBS), and then decreased again for the LPS 6h mouse model to a level comparable to that of the control group.

The track meandering index (FIG. 12E) represents the tendency of neutrophils to flow along one direction. A larger track meandering index indicates that target factors (neutrophils) in the blood stream flow linearly to a target location or along a particular direction to arrive at the location within the shortest time. As shown in FIG. 12E, the meandering index was about 0.5 a.u. for the control group (PBS), about 0.4 a.u. for the LPS 3h mouse model and about 0.2 a.u. for the LPS 6h mouse model. The track meandering index of neutrophils was smaller for the injury mouse models (LPS administration groups) as compared to the control group (PBS). The track meandering index at 6 hours after the LPS administration (LPS 6h mouse model) was decreased by about ½ as compared to at 3 hours after the LPS administration (LPS 3h mouse model).

From FIGS. 12B-12D, it was confirmed that track displacement length, track length and track velocity were increased in the LPS 3h mouse model among the dynamic behavior of neutrophils. This suggests that the motility of neutrophils in capillaries was increased at 3 hours after the LPS administration. However, the track displacement length, track length and track velocity were decreased in the LPS 6h mouse model because the motility of neutrophils was decreased due to aggravation of inflammation owing to microcirculatory disorder.

Also, as seen from FIG. 12E, the track meandering index was decreased in the order of the control group, the LPS 3h mouse model and the LPS 6h mouse model, which was influenced by the increased sequestration time and the arrest (or sequestration) characteristics of the neutrophils.

Taken together, the dynamic behavior of neutrophils shows that, during the early period of endotoxin-induced acute lung injury, neutrophils are activated and become motile inside the capillaries; however, in the late period, they are gradually arrested inside the capillaries. Accordingly, the method for providing information and the apparatus for diagnosis of microcirculatory disorder according to an aspect of the present disclosure can provide a plurality of motion images of neutrophils passing through capillaries by clearly visualizing the motion of neutrophils inside the capillaries and allow easy and convenient diagnosis of microcirculatory disorder in a subject by acquiring information about the motion of neutrophils.

### [Experimental Example 7] Investigation of Correlation Between Neutrophil Sequestration and Dead Space Formation

It was confirmed from Experimental Example 6 that neutrophils are sequestered inside capillaries due to acute lung injury. The entire process of dead space formation in the pulmonary microcirculation was investigated through intravital imaging of the ALI mouse model prepared in Example 1-2.

Real-Time Imaging and Time-Lapse Imaging

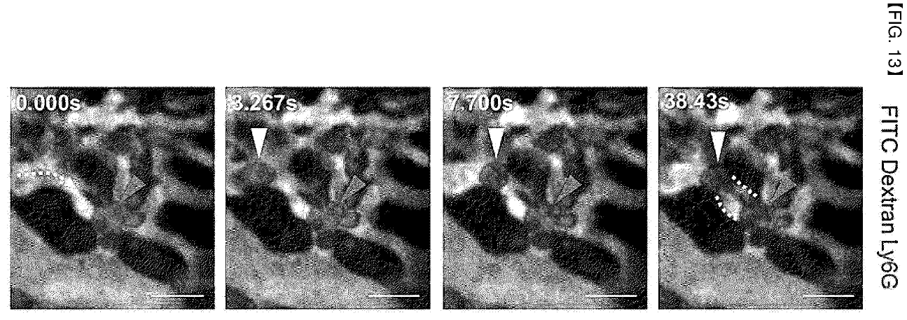

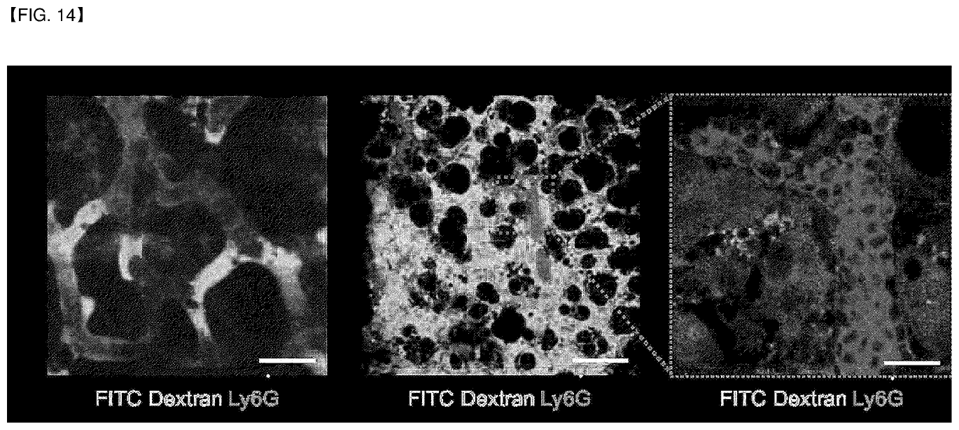

Specifically, the real-time images of FIGS. 13 and 14 were obtained by imaging the pulmonary microcirculation of neutrophils (Ly6G+ cells) of the ALI mouse model of Example 1-2 in real time according to the method of Experimental Example 4.

In addition, cluster formation by neutrophils (Ly6G+ cells) in the branching region of arterioles connected to capillaries was intravital imaged at a slow rate for 10 minutes using a customized video-rate laser scanning confocal microscopy system according to the same method as in Experimental Examples 1-1 and 1-2. As a result, the time-lapse images of FIG. 15 were obtained.

As shown in FIG. 13, in the capillaries, a circulating neutrophil became trapped on one side of the vessel in which the other side was already obstructed by another neutrophil. The flow stopped between the two neutrophils, thereby generating a dead space in the microcirculation. At some capillary sites, clusters of neutrophils where no flow was detected could be observed (FIG. 14). Such obstruction was not limited to the capillaries but was also observed in the branching regions of arterioles connected to the capillaries.

In addition, as shown in FIG. 15, over the course of 10 minutes of imaging of the motion of neutrophils in the pulmonary capillaries, it was observed that neutrophils blocked the branching sites and disturbed the microcirculation near the obstructed region.

Confirmation of Correlation Between Neutrophil Sequestration and Dead Space Formation Through Visualization of Functional Capillaries

To confirm the correlation between neutrophil sequestration and dead space formation, erythrocytes were stained to visualize the functional capillaries where the erythrocytes circulate smoothly. Specifically, the pulmonary microcirculation of the ALI mouse model of Example 2 having DiD-labeled erythrocytes was imaged at a slow rate for 10 minutes according to the method of Experimental Example 5 and the obtained images were processed by the method of Experimental Examples 1-1 and 1-2. The track path of the DiD-labeled erythrocytes was obtained in the same manner as in the neutrophil tracking of Experimental Example 5. The result is shown in FIG. 16. In FIG. 16, the white dashed circles indicate dead space in the microcirculation, and white arrows indicate the direction of blood flow. In FIG. 16, the scale bars are 100 μm.

As shown in FIG. 16, it was confirmed that neutrophil-induced capillary and arteriole obstruction generated dead space in the microcirculation because the erythrocytes did not move in the area where clusters were formed by neutrophils.

Accordingly, since the neutrophils sequestered inside capillaries due to microcirculatory disorder such as lung injury can form dead space, the method for providing information and the apparatus for diagnosis of microcirculatory disorder according to an aspect of the present disclosure can provide a plurality of motion images of neutrophils passing through capillaries by clearly visualizing the motion of neutrophils inside the capillaries and allow easy and convenient diagnosis of microcirculatory disorder in a subject by acquiring information about the motion of neutrophils.

### [Experimental Example 8] Relationship Between Neutrophil Sequestration and Reactive Oxygen Species (ROS)

It was confirmed in Experimental Example 6 that neutrophils in capillaries are sequestered due to acute lung injury. The effect of neutrophil sequestration on the release of reactive oxygen species (ROS) in situ was investigated for the ALI mouse model and the control group to which PBS was administered instead of LPS prepared in Example 1-2.

Specifically, DHE (dihydroethidium) staining was performed using high-dose DHE (10 mg/kg) to investigate the generation of reactive oxygen species according to the method previously used in intravital imaging researches (Finsterbusch M, Hall P, Li A, Devi S, Westhorpe C L, Kitching A R, Hickey M J. Patrolling monocytes promote intravascular neutrophil activation and glomerular injury in the acutely inflamed glomerulus. Proc Natl Acad Sci USA 2016: 113(35): E5172-5181). A stock solution was prepared by dissolving 15.7 mg of DHE in 1.5 mL of DMSO and was stored at −20° C. Then, after heating the DHE solution up to 60° C. to 10 mg/kg, followed by diluting in 50 μL of saline, it was immediately injected intravenously to the ALI mouse model and the control mouse model of Example 1-2 for intravital microscopy.

The generation of reactive oxygen species (ROS) was investigated 20 minutes after the injection of DHE. The number of neutrophils (ROS+Ly6G+) was determined by counting the cells with naked eyes or using the ImageJ program, or using the Spots function of the IMARIS program. In addition, the number of reactive oxygen species-generating neutrophils (ROS+Ly6G+) was determined from the neutrophils double positive for neutrophils (Ly6G+, red color) and DHE (blue color) using the Colocalization function of the IMARIS program.

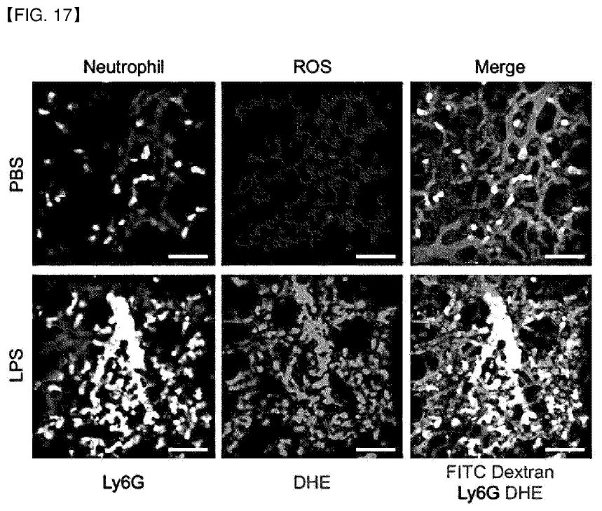

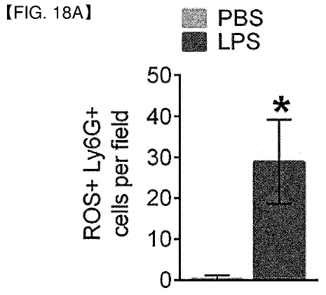

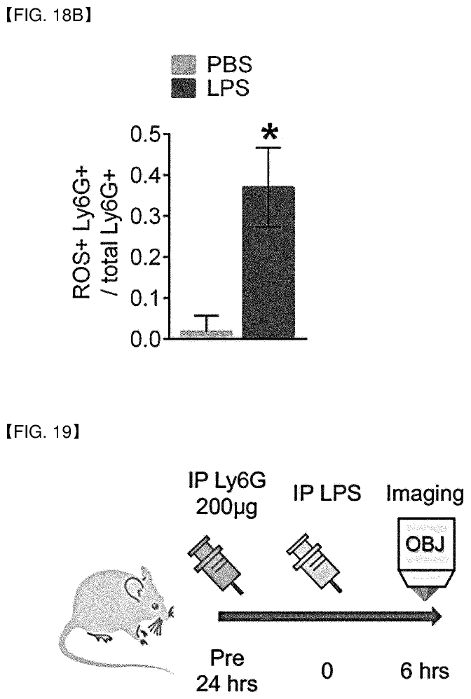

The result is shown in FIG. 17 and FIG. 18 (n (number of fields)=30, 10 FOV (field of view) per mouse, 3 mice per each group, *P<0.05, two-tailed t-test, data are means±s.d.). As shown in FIG. 17, the generation of reactive oxygen species in intravascular neutrophils was confirmed in situ.

In addition, as shown in FIG. 18A, when the number of reactive oxygen species-generating neutrophils (ROS+Ly6G+) per unit area (field, 512×512 μm) was compared for the control group (PBS) and the ALI mouse model (LPS), there was almost no reactive oxygen species-generating neutrophil in the control group, whereas as many as about 30 reactive oxygen species-generating neutrophils were found in the ALI mouse model. In addition, as shown in FIG. 18B, the proportion of reactive oxygen species-generating neutrophils (ROS+Ly6G+) in total neutrophils (Ly6G+) was increased to about 0.4 in the ALI mouse model (LPS) whereas it was almost close to 0 for the control group (PBS). That is to say, as seen from FIGS. 18A and 18B, whereas reactive oxygen species could not be detected from sequestered neutrophils in the control group (PBS), the number and proportion of reactive oxygen species-generating neutrophils were increased significantly in the ALI mouse model (LPS).

Through this, it was confirmed that, in contrast to the previous understanding that reactive oxygen species are produced by neutrophils at the site of inflammation, the production of reactive oxygen species is initiated at a much early stage because of the development of neutrophil entrapment in capillaries. The findings also imply that the entrapped neutrophils could release reactive oxygen species in situ, which could harm the endothelial cells and adjacent intravascular structure before extravasation.

Accordingly, since the neutrophils sequestered inside capillaries can produce reactive oxygen species during microcirculatory disorder such as lung injury can form dead space, the method for providing information and the apparatus for diagnosis of microcirculatory disorder according to an aspect of the present disclosure can provide a plurality of motion images of neutrophils passing through capillaries by clearly visualizing the motion of neutrophils inside the capillaries and allow easy and convenient diagnosis of microcirculatory disorder in a subject by acquiring information about the motion of neutrophils.

To summarize, the method for quantitation of microcirculation and the apparatus for measuring microcirculation according to an aspect of the present disclosure allow easier and more convenient quantification of microcirculation in a subject in vivo based on the functional capillary ratio, and allow accurate and fast diagnosis of microcirculatory disorder based on the quantification result.

### [Experimental Example 9] Imaging of Pulmonary Microcirculation in Neutrophil-Depleted Model

Pulmonary microcirculation was imaged in the same manner as in Experimental Example 1-1 except that the control mouse model (PBS) and the ALI mouse model (LPS) of Example 1-3 and the neutrophil-depleted models of Example 1-4 (N-Dep mouse model and N-Dep+LPS mouse model) were used as mouse models instead of the Tie2-GFP-ALI mouse model of Example 1-1.

After imaging the pulmonary microcirculation of the control mouse model (PBS) and the ALI mouse model (LPS) of Example 1-3 and the neutrophil-depleted models of Example 1-4 (N-Dep mouse model and N-Dep+LPS mouse model) using the imaging system described above, the obtained images were processed according to the image processing process described above. The result is shown in FIG. 20. Among the neutrophil-depleted models of Example 1-4, the N-Dep+LPS mouse model was imaged using the image system 6 hours after the injection of LPS. For the sepsis-induced acute lung injury mouse models, the functional capillary ratio (FCR; calculated as a ratio of functional capillary area to total capillary area) is decreased in the early stage of acute lung injury. As shown in FIG. 20, unlike the control group (PBS), the formation of dead space where erythrocytes cannot pass through capillaries and increased entrapment of neutrophils were found in the ALI mouse model (LPS). In contrast, the pulmonary microcirculatory disorder was improved in the neutrophil-depleted models (N-Dep mouse model and N-Dep+LPS mouse model) as the dead space formation and increased entrapment were decreased and FCR was increased.

### [Experimental Example 10] Investigation of Functional Capillary Ratio (FCR) of Neutrophil-Depleted Model

Comparison of Functional Capillary Ratio (FCR)

It was confirmed in Experimental Example 9 that pulmonary microcirculatory disorder is improved in the neutrophil-depleted models. In order to confirm it through quantified data, functional capillary imaging analysis was performed using the real-time movie of DiD-labeled red blood cell flowing through capillaries obtained using the imaging system and image processing described in Experimental Example 9. After splitting colors of the movie, sequential images of channels detecting DiD were processed by a median filter with a radius of 2 pixels to enhance the signal-to-noise ratio. Maximal intensity projection of 600-900 frames (20-30 seconds) was generated to show the functional capillary perfused by erythrocytes. The functional capillary ratio (FCR) was calculated according to Formula 1.

Functional capillary ratio=functional capillary area/total capillary area. [Formula 1]

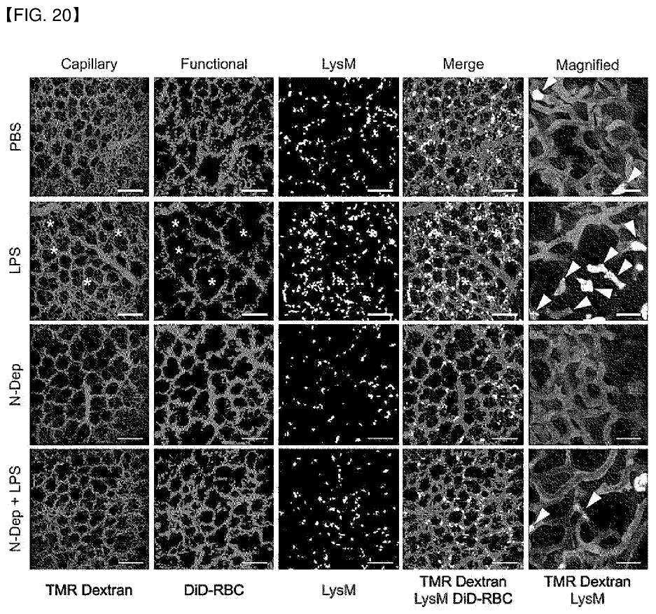

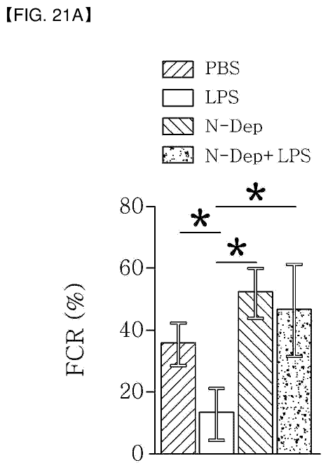

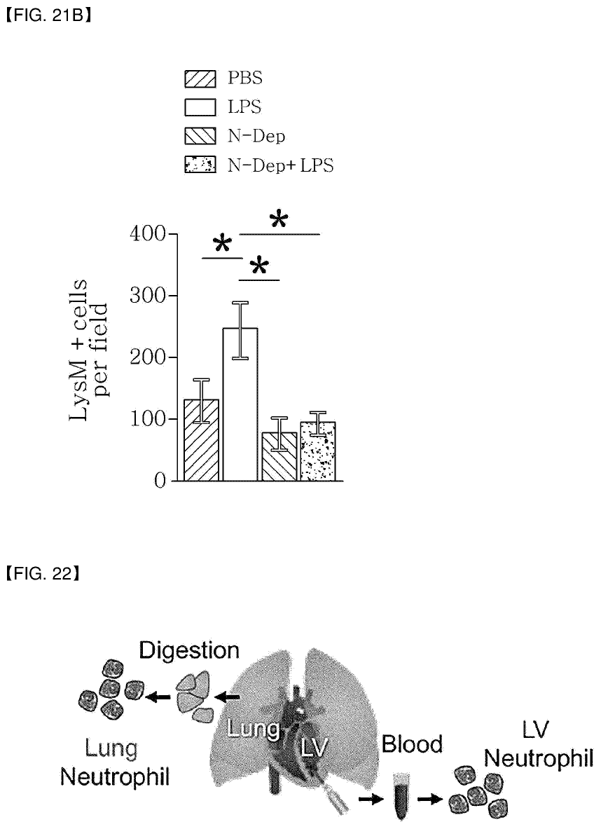

In Formula 1, the total capillary area means the vessel area detected by Tie2 or dextran signaling, and the functional capillary area means the area traveled by DiD-labeled erythrocytes. All image processing to calculate the functional capillary ratio was performed with ImageJ (https://imagej.nih.gov/ij/), and the result is shown in FIG. 21A (n (number of fields)=30, 10 FOV (field of view) per mouse, 3 mice per each group, P<0.05, one-way ANOVA with post-hoc Holm-Sidak's multiple comparison test, data are means±s.d.).

As shown in FIG. 21A, the FCR (%) of the ALI mouse model (LPS) of Example 1-3 was decreased by 50% or more as compared to the control (PBS) mouse model of Example 1-3, indicating the occurrence of pulmonary microcirculatory disorder due to lung injury. The neutrophil-depleted models of Example 1-4 (N-Dep mouse model and N-Dep+LPS mouse model) showed improvement in pulmonary microcirculatory disorder, with FCR (%) increased by about 3 times or more as compared to the ALI mouse model (LPS) of Example 1-3.

Histological Analysis

Histological analysis was performed to compare the number of neutrophils per unit area (512×512 μm) of the control mouse model (PBS) and the ALI mouse model (LPS) of Example 1-3 and the neutrophil-depleted models of Example 1-4 (N-Dep mouse model and N-Dep+LPS mouse model).

Specifically, lung tissues were harvested after intravital imaging of the mouse models. The tissues were perfused and fixed with 4% paraformaldehyde and then further fixed overnight in 4% paraformaldehyde. For H&E (hematoxylin and eosin) staining, the fixed tissues were processed using standard procedures, embedded in paraffin and then sliced into 4-μm sections, followed by conventional H&E staining. The result is shown in FIG. 21B (n (number of fields)=30, 10 FOV (field of view) per mouse, 3 mice per each group, P<0.05, one-way ANOVA with post-hoc Holm-Sidak's multiple comparison test, data are means±s.d.). FIG. 21B shows the number of neutrophils (cells/field) per unit area (512×512 μm).

In addition, although the number of neutrophils (LysM+ cells) was decreased to a certain level during the preparation of the neutrophil-depleted models of Example 1-4, as shown in FIG. 21B, the magnified images of FIG. 20 showed that the neutrophils were not completely depleted due to the remnant LysM+ cells, which were presumably alveolar macrophages in the extravascular space.

To summarize the results of FIG. 21A and FIG. 21B, it can be seen that the decreased number of LysM+ cells, mostly intravascular neutrophils, leads to an improved functional capillary ratio (FCR) in pulmonary microcirculation. In addition, it can be seen that the neutrophils function as the main components of aggregates and the primary blockers of low in the pulmonary microcirculation during systemic inflammation.

### [Experimental Example 11] Identification of Target for Neutrophils in Capillaries for Prevention or Treatment of Lung Injury

Although it was confirmed in Experimental Example 10 that neutrophil depletion in capillaries can prevent or treat lung injury by alleviating microcirculatory disorder, the neutrophil depletion strategy is not feasible clinically. Therefore, to identify the target for sequestered neutrophils for prevention or treatment of lung injury, experiment was conducted as follows. It was hypothesized that the integrin expression pattern of the neutrophils in the left ventricle that had passed through pulmonary capillaries would be different from the pattern of the neutrophils in the lung.

Flow Cytometry

For investigation of the integrin expression of left ventricle-derived neutrophils and lung-derived neutrophils, neutrophils were isolated from the left ventricle (LV) and lung of the control (PBS) mouse model and the ALI mouse model (LPS) of Example 1-3, respectively, and flow cytometry analysis was conducted for the isolated neutrophils.

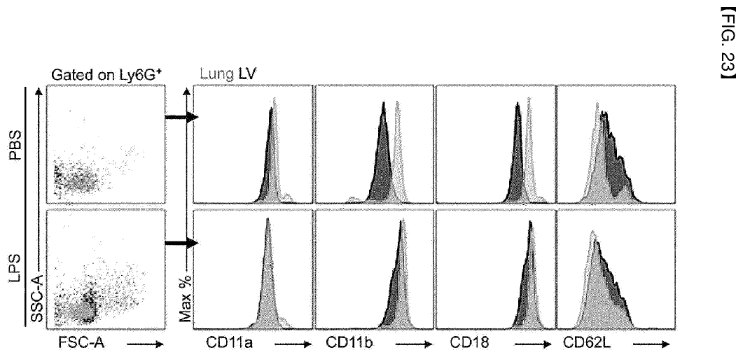

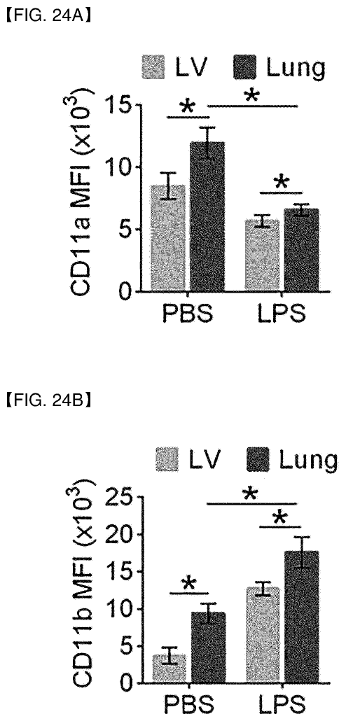

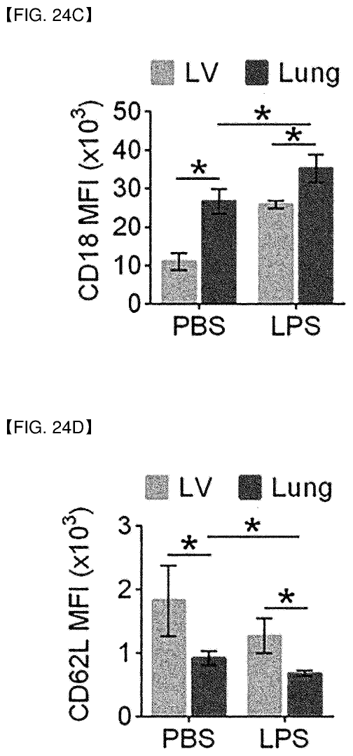

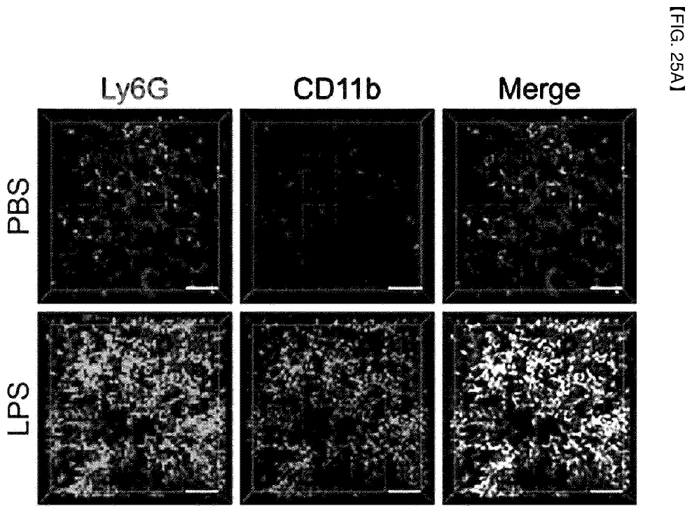

First, in order to isolate pulmonary sequestered neutrophils, the lung was harvested and digested without perfusion. The lung was placed in PBS solution, minced, filtered through a 40-μm filter and stained at 4° C. for 30 minutes. Meanwhile, for isolation of neutrophils from left ventricle, 100 μL of blood was taken from the left ventricle of the mouse model using a syringe and 1.0×10⁶ neutrophils were isolated using a flow cytometer (FACS, BD, LSRFortessa™) after hemolyzing erythrocytes. Ly6G-FITC (1A8, 551460, BD Biosciences), CD11a-BV510 (M17/4, 563669, BD Biosciences) CD11b-PE-Cy7 (M1/70, 552850, BD Biosciences), CD18-APC (C71/16, 562828, BD Biosciences), CD62L (MEL-14, 560514, BD Biosciences) and Viability Dye eFluor 506 (65-0866-14, ThermoFisher Scientific) were used as clonal antibodies and the stained cells were analyzed with an LSR Fortessa flow cytometer (BD Biosciences). Then, flow cytometry was performed for the two groups of neutrophils gated on Ly6G+ using Flowjo (FlowJo, LLC). The result is shown in FIG. 23 and FIGS. 24A-24D (n (number of mice)=5 per each group, *P<0.05, Mann-Whitney test, MFI: mean fluorescence intensity, data are means±s.d.).

From FIG. 23 and FIGS. 24A-24D, it was confirmed that the expression level of CD11b and CD18 in neutrophils is higher in the ALI mouse model (LPS) of Example 1-3 as compared to that in the control group (PBS) of Example 1-3 and that the expression level of CD11b and CD18 is higher in the lung-derived neutrophils than the left ventricle-derived neutrophils for the same mouse model (n=5 per each group, *P<0.05, Mann-Whitney test, MFI: mean fluorescence intensity, data are means±s.d.).

Intravital Imaging

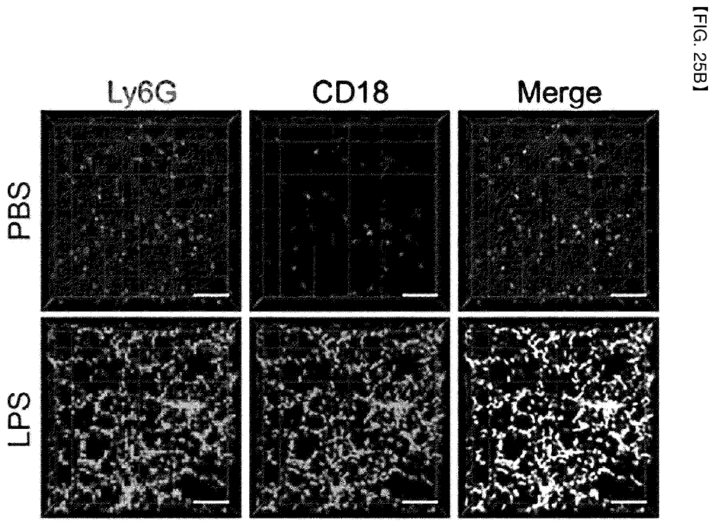

The integrin in the sequestered neutrophils of the control (PBS) mouse model of Example 1-3 and the ALI mouse model (LPS) was imaged and then processed according to the method of Experimental Example 9. The result of visualizing neutrophils and the expression of CD11b and CD18 on the surface of the neutrophils in vivo is shown in FIG. 25A and FIG. 25B, respectively.

As shown in FIG. 25A and FIG. 25B, for the control group (PBS), the number of neutrophils was very small and CD11b and CD18 were hardly expressed on the surface of the neutrophils. In contrast, for the ALI mouse model (LPS), a very large number of neutrophils were observed and the expression level of CD11b and CD18 on the surface of the neutrophils was very high.

Comparison of Number of Neutrophils Expressing CD11b or CD18

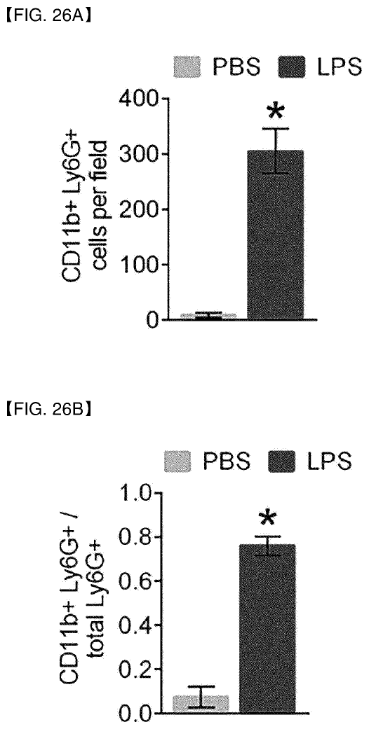

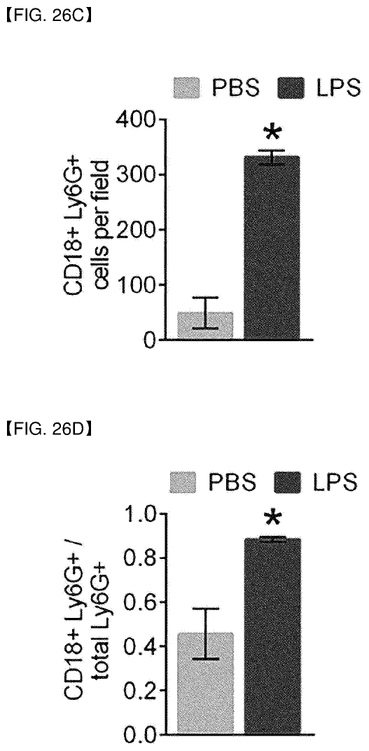

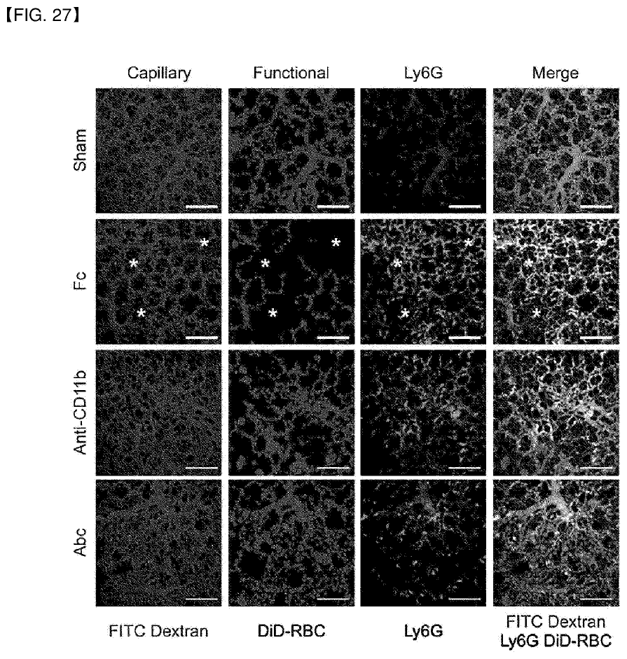

The number of neutrophils expressing CD11 b or CD18 was compared for the ALI mouse model (LPS) and control mouse model (PBS) of Example 1-3, and the result is shown in FIGS. 26A-26D (n (number of fields)=9, 3 FOV (field of view) per mouse, 3 mice per each group, *P<0.05, Mann-Whitney test, data are means±s.d.)). In FIG. 26A and FIG. 26C, the field means unit area (512×512 μm).

As shown in FIGS. 26A-26D, whereas the number of neutrophils expressing CD11 b on the surface unit area (CD11 b+Ly6G+ cells per field) was almost 0 for the control group (PBS), it was about 300 for the ALI mouse model (LPS) (FIG. 26A). In addition, the number of neutrophils expressing CD18 on the surface unit area (CD18+Ly6G+ cells per field) was about 40 for the control group (PBS), but about 330 for the ALI mouse model (LPS), which was about 8 times larger (FIG. 26C). The ratio of the neutrophils expressing CD11b on the surface to the total neutrophils (CD11b+Ly6G+/total Ly6G+) was about 0.05 for the control group (PBS), but 0.8 for the ALI mouse model (LPS), which was about 16 times larger (FIG. 26B). The ratio of the neutrophils expressing CD18 on the surface to the total neutrophils (CD18b+Ly6G+/total Ly6G+) was about 0.4 for the control group (PBS), but 0.9 for the ALI mouse model (LPS), which was about 2 times or larger (FIG. 26D). Through this, it can be seen that the expression level of CD11b and CD18 is highly increased on the surface of the neutrophils due to lung injury.

From the above results, it was confirmed that the expression level of Mac-1 (CD11b/CD18) integrin is increased in the neutrophils sequestered due to lung injury as compared to the neutrophils circulating in the microcirculation.

### [Experimental Example 12] Investigation of Effect of Mac-1 Inhibition on Alleviation of Lung Injury

It was confirmed in Experimental Example 11 that the expression level of Mac-1 (CD11b/CD18) integrin is increased in the neutrophils sequestered in the lung due to lung injury. In this example, it was investigated whether lung injury, particularly microcirculatory disorder, is improved by a Mac-1 inhibitor against the expression or activity in a Mac-1-inhibited mouse model prepared in Experimental Example 12-1. The CLP model of Example 1-3 was used as a sepsis model to extend the above findings to a polymicrobial sepsis model.

### [Experimental Example 12-1] Preparation of Mac-1-Inhibited Mouse Model

In order to investigate the effect of the inhibition of Mac-1 activity in lung injury, a mouse model with the activity of Mac-1 inhibited (hereinafter, Mac-1-inhibited model) was prepared. Specifically, the anti-Mac-1 model with the activity of Mac-1 inhibited was prepared by intraperitoneally injecting anti-CD11b antibody (5 mg/kg, Clone M_{1/70}, 553307, BD Biosciences) 1 hour after the preparation of the CLP mouse model of Example 1-3 and 5 hours before intravital. In addition, an abciximab model was prepared as another Mac-1-inhibited model by injecting abciximab (10 mg/kg, Clotinab, ISU Abxis) to the CLP mouse model of Example 1-3 in the same manner as the preparation of the anti-Mac-1 model.

### [Experimental Example 12-2] Investigation of Effect of Mac-1 Inhibition on Alleviation of Pulmonary Microcirculatory Disorder

The pulmonary microcirculation of the CLP mouse model of Example 1-3 (Fc in FIGS. 27 and 28) and the anti-Mac-1 mouse model of (Anti-CD11 b in in FIGS. 27 and 28), abciximab model (Abc in FIGS. 27 and 28) and normal (sham) mouse model (Sham in FIGS. 27 and 28) of Experimental Example 12-1 was imaged in the same manner as in Experimental Example 9. The functional capillary ratio (FCR) was measured therefrom and microcirculation was quantified by histological analysis in the same manner as in Experimental Example 10, and the result is shown in FIG. 27 and FIG. 28 (n (number of fields)=14-25, 3 mice per each group, *P<0.05, two-tailed t-test, data are means±s.d.).

As shown in FIG. 27, unlike the normal group (Sham), dead space where erythrocytes cannot pass through capillaries was formed in the CLP mouse model (Fc). But, as the Mac-1 activity was inhibited (anti-Mac-1 mouse model and abciximab mouse model), pulmonary microcirculatory disorder was alleviated as the dead space was decreased and the functional capillaries through which erythrocytes can flow smoothly are increased.

Also, as shown in FIG. 28A, the FCR (%) of the CLP mouse model (Fc) was decreased by 50% or more as compared to the normal group (Sham), indicating that pulmonary microcirculatory disorder occurred due to lung injury. The Mac-1-inhibited models (anti-Mac-1 mouse model and abciximab mouse model) showed alleviated pulmonary microcirculatory disorder with the FCR (%) increased by about 2 times or more as compared to the CLP mouse model (Fc).

The result of histological analysis also showed that the number of sequestered neutrophils (Ly6G+ cells) was increased for the CLP mouse model (Fc) as compared to the normal group (Sham) and then was recovered to a level comparable to that of the normal group by inhibition of Mac-1 (anti-Mac-1 mouse model and abciximab mouse model), as shown in FIG. 27 and FIG. 28B.

### [Experimental Example 12-3] Investigation of Alleviation of Pulmonary Microcirculatory Disorder Before and After Mac-1 Inhibition

It was confirmed in Experimental Example 12-2 that microcirculatory disorder can be alleviated by inhibiting the expression or activity of Mac-1. This was confirmed again by investigating the effect before and after administration of abciximab.

Specifically, pulmonary microcirculation was imaged 6 hours after preparation of the CLP mouse model of Example 1-3 (hereinafter, pre-Abc mouse model) in the same manner as in Experimental Example 9, and functional capillary ratio (FCR) was measured in the same manner as in Experimental Example 10. Then, after administering abciximab to the CLP mouse model in the same manner as in Experimental Example 12-1 (hereinafter, post-Abc mouse model), pulmonary microcirculation was imaged and functional capillary ratio (FCR) was measured 30 minutes later. The result is shown in FIG. 29 and FIG. 30 (n (number of fields)=20 and 24, 6-8 FOV (field of view) per mouse, 3 mice per each group, *P<0.05, two-tailed t-test, data are means±s.d.).

As shown in FIG. 29 and FIG. 30, for the lung injury mouse model (CLP mouse model) before abciximab administration, the ratio of functional capillary to the total capillary (FCR) was smaller than 20%, indicating that many erythrocytes cannot pass through capillaries. However, when the expression or activity of Mac-1 was inhibited by administering abciximab, the number of erythrocytes passing through capillaries was increased rapidly, with the functional capillary ratio (FCR) increased by about 2 times or more.

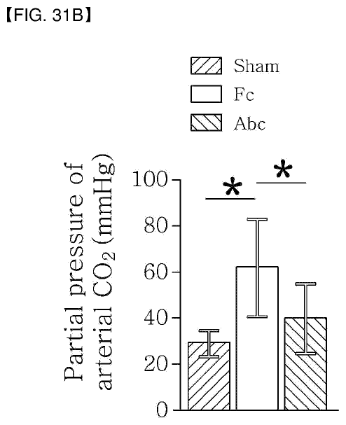

In addition, arterial blood gas analysis was performed to assess the oxygen partial pressure and carbon dioxide partial pressure in the arterial blood of the pre-Abc mouse model and the post-Abc mouse model. Specifically, a 1-mL syringe with a 22-gauge needle was coated with heparin and introduced into the left ventricle of the heart of the normal mouse model (Sham) (n=8), pre-Abc mouse model (Fc) (n=10) and post-Abc mouse model (Abc) (n=6). Then, about 200 μL of blood was sampled and analyzed with an i-STAT handheld blood analyzer (G3 cartridge, Abbott Point of Care Inc.). The mice were euthanized in a CO₂ chamber right after the blood sampling. The arterial blood gas analysis result is shown in FIG. 31A and FIG. 31B (*P<0.05, Kruskal-Wallis test with post-hoc Dunn's multiple comparison test, data are means±s.d.).

As shown in FIGS. 31A and 31B, the oxygen partial pressure in the arterioles was decreased (FIG. 31A) and the carbon dioxide partial pressure was increased (FIG. 31B) in the CLP mouse model (Fc, pre-Abc mouse model) as compared to the normal group (Sham). It was confirmed that the decrease of functional capillary ratio in the lung injury mouse model (CLP mouse model) was the result of hypoxemia and hypercapnia. The pulmonary microcirculatory disorder caused by hypoxia and hypercapnia was alleviated by the inhibition of the expression or activity of Mac-1 through administration of abciximab, which was confirmed by the change in the oxygen partial pressure and carbon dioxide partial pressure in the arterioles of the post-Abc mouse model (Abc) comparable to that of the normal group (FIGS. 31A and 31B). Through this, it can be seen that pulmonary microcirculatory disorder in a subject suffering from pulmonary microcirculatory disorder can be alleviated by increasing gas exchange through inhibition of the expression or activity of Mac-1.

Accordingly, the composition containing a Mac-1 inhibitor against the expression or activity in neutrophils according to an aspect of the present disclosure has a superior effect of preventing or treating lung injury by alleviating microcirculatory disorder in the lung.

1. A method for quantitation of microcirculation in a subject, comprising:
obtaining a plurality of motion images of first target factors over time in a blood stream passing through the capillaries of the subject;
measuring functional capillary area in which the first target factors move in the blood stream from the plurality of motion images; and
calculating functional capillary ratio (FCR) according to Formula 1:
Functional capillary ratio=functional capillary area/total capillary area. [Formula 1]

2. The method for quantitation of microcirculation according to claim 1, wherein the first target factors in the blood stream are one or more selected from a group consisting of leukocytes, erythrocytes, blood platelets and lymphocytes.

3. The method for quantitation of microcirculation according to claim 2, wherein the first target factors in the blood stream are fluorescence-stained first target factors in the blood stream.

4. The method for quantitation of microcirculation according to claim 1, wherein the plurality of motion images over time are a plurality of images imaged at a frame rate of 1-900 frames/second.

5. (canceled)

6. The method for quantitation of microcirculation according to claim 1, wherein the measurement of functional capillary area is to measure functional capillary area by identifying the same target factors from the plurality of motion images.

7. The method for quantitation of microcirculation according to claim 1, wherein the measurement of functional capillary area is calculated by measuring the area traveled by the first target factors in the blood stream from the change in location over time.

8-9. (canceled)

10. A method for providing information for diagnosis of microcirculatory disorder in a subject, comprising acquiring information for diagnosing microcirculatory disorder in the subject from the functional capillary ratio (FCR) calculated by the method for quantitation of microcirculation in a subject according to claim 1.

11. (canceled)

12. A method for providing information for diagnosis of microcirculatory disorder, comprising:
obtaining a plurality of motion images of second target factors over time in a blood stream flowing through the capillaries of the subject;
analyzing one or more dynamic element selected from a group consisting of sequestration time, track displacement length, track length, track velocity and track meandering index of the second target factors in the blood stream from the plurality of motion images; and
acquiring information for diagnosis of microcirculatory disorder in the subject from the dynamic element analysis result.

13. The method for providing information according to claim 12, wherein the second target factors in the blood stream are neutrophils.

14. The method for providing information according to claim 13, wherein an antibody specific for neutrophils is bound to the neutrophils, and the antibody is labeled with a fluorophore.

15. The method for providing information according to claim 12, wherein the plurality of motion images over time are imaged at a frame rate of 1-900 frames/second.

16. The method for providing information according to claim 15, wherein the plurality of images are imaged by a confocal scanning laser microscope.

17. The method for providing information according to claim 12, wherein the analysis of the dynamic element is conducted by identifying the same target factors from the plurality of motion images.

18. The method for providing information according to claim 12, wherein the information for diagnosis of microcirculatory disorder in the subject is determined to be microcirculatory disorder if the sequestration time of the second target factors in the blood stream is 5 minutes or longer.

19. The method for providing information according to claim 12, wherein the information for diagnosis of microcirculatory disorder in the subject is determined to be microcirculatory disorder if the track meandering index of the second target factors in the blood stream is 0.4 a.u. or lower.

20. The method for providing information according to claim 12, wherein
the plurality of motion images are two or more sets of plurality of motion image of the second target factors in the blood stream over time imaged with an interval of 2 hours or longer,
the dynamic element is one or more selected from a group consisting of the track displacement length, track length and track velocity of the second target factors in the blood stream, and
the analysis of the dynamic element is conducted by analyzing the dynamic element from the two or more sets of plurality of motion image sequentially in time.

21. The method for providing information according to claim 20, wherein the information for diagnosis of microcirculatory disorder in the subject is determined to be microcirculatory disorder if the dynamic element analyzed from the two or more sets of plurality of motion image decreases over time.

22. The method for providing information according to claim 12, wherein the method further comprises detecting whether reactive oxygen species are generated in the second target factors in the blood stream flowing through the capillaries of the subject.

23. The method for providing information according to claim 22, wherein the information for diagnosis of microcirculatory disorder in the subject is determined to be microcirculatory disorder if reactive oxygen species are generated in the target factors.

24. The method for providing information according to claim 12, wherein the capillaries of the subject are capillaries of one or more selected from a group consisting of the lung, kidney, skin and eye of the subject.

25-41. (canceled)
